(19) United States
(12) Patent Application Publication
(10) Pub. No.: US 2024/0100351 A1
(43) Pub. Date: Mar. 28, 2024

(54) ELECTROENCEPHALOGRAM SCORING AND REPORTING FOR TRANSCRANIAL MAGNETIC STIMULATION

(71) Applicant: Wave Neuroscience, Inc., Newport Beach, CA (US)

(72) Inventors: James William Phillips, Fountain Valley, CA (US); Alexander Joseph Ring, Irvine, CA (US)

(21) Appl. No.: 18/477,517

(22) Filed: Sep. 28, 2023

Related U.S. Application Data
(60) Provisional application No. 63/377,514, filed on Sep. 28, 2022.

(51) Int. Cl. A61N 2/00; A61B 5/00; A61B 5/374

(52) U.S. Cl. CPC A61N 2/006; A61B 5/374; A61B 5/7203; A61B 5/7257; A61B 5/384

(57) ABSTRACT

A method for scoring and reporting electroencephalogram (EEG) data for use in transcranial magnetic stimulation (TMS) therapy. The method may include removing artifacts from the EEG data and determining EEG metrics from the EEG data. The method may further include determining a Brain Synchrony Index from the EEG metrics by applying a predetermined transfer function to the EEG metrics, and reporting the Brain Synchrony Index graphically.

### CROSS-REFERENCE TO RELATED APPLICATIONS

This application claims priority to and the benefit of U.S. Provisional Patent Application No. 63/377,514, titled “ELECTROENCEPHALOGRAM SCORING AND REPORTING FOR TRANSCRANIAL MAGNETIC STIMULATION” and filed on Sep. 28, 2022, the entire contents of which is hereby incorporated by reference herein.

### FIELD

The disclosure relates to methods for scoring and reporting electroencephalogram (EEG) data for use in transcranial magnetic stimulation (TMS) treatment.

### BACKGROUND

Mental disorders generate serious problems for the affected people, their families, and society. Currently, psychiatrists and neurophysiologists treat these disorders with a variety of medications, many of which have significant negative side effects. Treatment of these disorders with magnetic fields may generate positive therapeutic responses.

For example, magnetic fields may be generated by transcranial magnetic stimulation (TMS). TMS is a non-invasive procedure that typically uses magnetic field pulses or waves to stimulate nerve cells and neuronal circuitry in the brain to improve certain mental disorders such as schizophrenia, obsessive compulsive disorder (OCD), and depression.

The electrical activity of a living person's brain can be depicted on an electroencephalogram (EEG). An EEG is an electrical recording of the activity of the brain taken from the scalp. Electroencephalography measures voltage fluctuations resulting from current flow within neurons, typically of the brain. On an EEG, the voltage at various locations on the scalp are recorded over time. The time variant signals recorded on the EEG may be known as “brainwaves.” Brainwaves are complicated signals that can be analyzed in different ways.

### SUMMARY

The disclosure relates generally to a system or method for providing transcranial magnetic stimulation (TMS) based on electroencephalogram (EEG) data.

It is to be understood that any combination of features from the methods disclosed herein and/or from the systems and/or devices disclosed herein may be used together, and/or that any features from any or all of these aspects may be combined with any of the features of the embodiments and/or examples disclosed herein to achieve the benefits as described in this disclosure.

At least one aspect of the present disclosure is directed to a method for scoring and reporting electroencephalogram (EEG) data for use in transcranial magnetic stimulation (TMS) therapy. The method includes removing artifacts from the EEG data, determining EEG metrics from the EEG data, and determining a Brain Synchrony Index from the EEG metrics by applying a predetermined transfer function to the EEG metrics.

In some embodiments, the method includes reporting at least one of a current user frequency, an optimal brain range, and an interference score via the user interface. In some embodiments, the EEG data includes a set of channels, wherein each channel corresponds to a scalp electrode used to record the EEG data, and wherein the set of channels includes at least one channel. In some embodiments, at least one set of channels includes a plurality of channels that correspond to scalp electrodes to sense a region on the scalp. In some embodiments, removing artifacts from the EEG data includes filtering out at least one of noise artifacts and eye-blink artifacts.

In some embodiments, the EEG metrics include an intrinsic alpha frequency based on a dominant brainwave frequency in an alpha EEG band. In some embodiments, determining the EEG metrics includes finding and labeling all alpha bursts in the EEG data. In some embodiments, determining the EEG metrics further includes determining an alpha frequency corresponding to each of the alpha bursts. In some embodiments, the EEG data includes a set of channels, wherein each channel corresponds to a scalp electrode used to record the EEG data, wherein the set of channels includes at least one channel, and wherein the determining the EEG metrics further includes determining an alpha frequency variability for the set of channels.

In some embodiments, the EEG data includes a set of channels, wherein each channel corresponds to a scalp electrode used to record the EEG data, wherein the set of channels includes at least one channel, wherein the determining the EEG metrics further includes determining an alpha duration for the set of channels, and wherein the alpha duration for the set of channels is determined by summing the duration of all of the alpha bursts corresponding to the set of channels. In some embodiments, determining the EEG metrics further includes determining an EEG length based on a time duration of the EEG data. In some embodiments, determining the EEG metrics further includes determining an alpha prevalence from a ratio of the alpha duration and the EEG length.

In some embodiments, the predetermined transfer function is generated from at least one factor analysis performed on a plurality of EEG recordings corresponding to a plurality of different users. In some embodiments, the plurality of EEG recordings includes EEG recordings collected before and after TMS treatment. In some embodiments, the at least one factor analysis includes an exploratory factor analysis (EFA) and a confirmatory factor analysis (CFA).

Another aspect of the present disclosure is directed to a method for generating a transfer function for determining a Brain Synchrony Index. The method includes receiving an electroencephalogram (EEG) dataset including a plurality of EEG recordings corresponding to a plurality of individuals, performing a first factor analysis on at least a portion of the EEG dataset to identify one or more impacted variables, performing a second factor analysis on at least a portion of the EEG dataset to validate the one or more impacted variables, and generating the transfer function based on the one or more impacted variables validated by the second factor analysis.

In some embodiments, generating the transfer function based on the one or more impacted variables validated by the second factor analysis includes using an optimization algorithm to derive the transfer function from the one or more impacted variables. In some embodiments, the first factor analysis is an exploratory factor analysis (EFA). In some embodiments, the second factor analysis is a confirmatory factor analysis (CFA). In some embodiments, the one or more impacted variables correspond to variables that are impacted by stimulation treatment.

In some embodiments, the one or more impacted variables correspond to variables that are impacted by one or more diseases. In some embodiments, the method includes splitting the EEG dataset into a training dataset and a validation dataset. In some embodiments, performing the first factor analysis on at least a portion of the EEG dataset includes performing the first factor analysis on the training dataset. In some embodiments, performing the second factor analysis on at least a portion of the EEG dataset includes performing the second factor analysis on the validation dataset.

Another aspect of the present disclosure is directed to a method of treating a subject. The method includes measuring EEG data associated with the subject's brain, determining EEG metrics from the EEG data, determining a Brain Synchrony Index from the EEG metrics by applying a predetermined transfer function to the EEG metrics, selecting a stimulation frequency based on the Brain Synchrony Index, and providing a stimulation treatment at the stimulation frequency close to a head of the subject to influence the Brain Synchrony Index of the subject.

In some embodiments, the method includes reporting the Brain Synchrony Index graphically via a user interface. In some embodiments, the stimulation treatment is transcranial magnetic stimulation (TMS) therapy. In some embodiments, the method includes removing artifacts from the EEG data. In some embodiments, the method includes measuring second EEG data associated with the subject's brain following the stimulation treatment, determining second EEG metrics from the second EEG data, and determining a second Brain Synchrony Index from the second EEG metrics by applying the predetermined transfer function to the second EEG metrics. In some embodiments, the method includes reporting a difference between the Brain Synchrony Index and the second Brain Synchrony Index graphically via a user interface.

Another aspect of the present disclosure is directed to a system for providing treatment to a subject. The system includes at least one stimulation source, at least one memory storing computer-executable instructions, and at least one processor for executing the instructions stored on the memory. The execution of the instructions programs the at least one processor to perform operations that include measuring EEG data associated with the subject's brain, determining EEG metrics from the EEG data, determining a Brain Synchrony Index from the EEG metrics by applying a predetermined transfer function to the EEG metrics, selecting a stimulation frequency based on the Brain Synchrony Index, and providing a stimulation treatment at the stimulation frequency close to a head of the subject to influence the Brain Synchrony Index of the subject.

Another aspect of the present disclosure is directed to a method of treating a subject. The method incudes measuring first EEG data associated with at least one functional network of the subject's brain while the subject is performing a functional task associated with the at least one functional network, determining a first intrinsic frequency of the at least one functional network from the first EEG data, selecting a stimulation frequency based on the first intrinsic frequency, and providing a stimulation treatment at the stimulation frequency close to a head of the subject to influence the first intrinsic frequency of the at least one functional network.

In some embodiments, measuring the first EEG data associated with the at least one functional network includes receiving EEG data from a plurality of EEG channels and applying weights to the plurality of EEG channels to emphasize one or more EEG channels associated with the at least one functional network. In some embodiments, measuring the first EEG data associated with the at least one functional network includes receiving EEG data from a plurality of EEG channels and filtering the plurality of EEG channels to emphasize one or more EEG channels associated with the at least one functional network. In some embodiments, measuring the first EEG data associated with the at least one functional network includes receiving the first EEG data from a plurality of EEG corresponding to scalp electrodes positioned close to a physical location of the at least one functional network. In some embodiments, providing the stimulation treatment at the stimulation frequency close to the head of the subject includes providing the stimulation treatment close to a physical location of the at least one functional network.

In some embodiments, the method includes measuring second EEG data associated with the at least one functional network of the subject's brain following the stimulation treatment, determining a second intrinsic frequency of the at least one functional network from the second EEG data, and comparing the second intrinsic frequency to the first intrinsic frequency. In some embodiments, the second EEG data is measured while the subject is performing a functional task associated with the at least one functional network. In some embodiments, the second EEG data is measured while the subject is at rest.

In some embodiments, the method includes selecting a second stimulation frequency based on the second intrinsic frequency, and providing a stimulation treatment at the second stimulation frequency close to the head of the subject to influence the second intrinsic frequency of the at least one functional network. In some embodiments, the method includes selecting a second stimulation frequency based on a result of the comparison between the first and second intrinsic frequencies, and providing a stimulation treatment at the second stimulation frequency close to the head of the subject to influence the second intrinsic frequency of the at least one functional network. In some embodiments, the method includes measuring second EEG data associated with the at least one functional network of the subject's brain during the stimulation treatment, determining a second intrinsic frequency of the at least one functional network from the second EEG data, and comparing the second intrinsic frequency to the first intrinsic frequency.

In some embodiments, the second EEG data is measured while the subject is performing a functional task associated with the at least one functional network. In some embodiments, the method includes determining first EEG metrics from the first EEG data and determining a Brain Synchrony Index from the EEG data by applying a predetermined transfer function to the first EEG data.

Another aspect of the present disclosure is directed to a method of treating a subject. The method includes instructing the subject to visualize a functional task associated with at least one functional network of the subject's brain, measuring first EEG data associated with at least one functional network of the subject's brain, determining a first intrinsic frequency of the at least one functional network from the first EEG data, selecting a stimulation frequency based on the first intrinsic frequency, and providing a stimulation treatment at the stimulation frequency close to a head of the subject to influence the first intrinsic frequency of the at least one functional network.

In some embodiments, measuring the first EEG data associated with the at least one functional network includes receiving EEG data from a plurality of EEG channels and applying weights to the plurality of EEG channels to emphasize one or more EEG channels associated with the at least one functional network. In some embodiments, measuring the first EEG data associated with the at least one functional network includes receiving EEG data from a plurality of EEG channels and filtering the plurality of EEG channels to emphasize one or more EEG channels associated with the at least one functional network. In some embodiments, measuring the first EEG data associated with the at least one functional network includes receiving the first EEG data from a plurality of EEG corresponding to scalp electrodes positioned close to a physical location of the at least one functional network.

In some embodiments, providing the stimulation treatment at the stimulation frequency close to the head of the subject includes providing the stimulation treatment close to a physical location of the at least one functional network. In some embodiments, the method includes measuring second EEG data associated with the at least one functional network of the subject's brain following the stimulation treatment, determining a second intrinsic frequency of the at least one functional network from the second EEG data, and comparing the second intrinsic frequency to the first intrinsic frequency. In some embodiments, the second EEG data is measured after the subject has been instructed to visualize a functional task associated with the at least one functional network. In some embodiments, the second EEG data is measured while the subject is at rest.

In some embodiments, the method includes selecting a second stimulation frequency based on the second intrinsic frequency, and providing a stimulation treatment at the second stimulation frequency close to the head of the subject to influence the second intrinsic frequency of the at least one functional network. In some embodiments, the method includes selecting a second stimulation frequency based on a result of the comparison between the first and second intrinsic frequencies, and providing a stimulation treatment at the second stimulation frequency close to the head of the subject to influence the second intrinsic frequency of the at least one functional network.

In some embodiments, the method includes measuring second EEG data associated with the at least one functional network of the subject's brain during the stimulation treatment, determining a second intrinsic frequency of the at least one functional network from the second EEG data, and comparing the second intrinsic frequency to the first intrinsic frequency. In some embodiments, the second EEG data is measured after the subject has been instructed to visualize a functional task associated with the at least one functional network. In some embodiments, the method includes determining first EEG metrics from the first EEG data, and determining a Brain Synchrony Index from the EEG data by applying a predetermined transfer function to the first EEG data.

Another aspect of the present disclosure is directed to a method for scoring and reporting electroencephalogram (EEG) data for use in transcranial magnetic stimulation (TMS) therapy substantially as shown and described.

Another aspect of the present disclosure is directed to a computer-implemented method substantially as hereinbefore described with reference to any of the examples and/or to any of the accompanying drawings.

Another aspect of the present disclosure is directed to a computing system including one or more processors and one or more memories configured to perform operations substantially as hereinbefore described with reference to any of the examples and/or to any of the accompanying drawings.

Another aspect of the present disclosure is directed to a computer program product residing on a computer readable storage medium having a plurality of instructions stored thereon which, when executed across one or more processors, causes at least a portion of the one or more processors to perform operations substantially as hereinbefore described with reference to any of the examples and/or to any of the accompanying drawings.

Another aspect of the present disclosure is directed to a device configured substantially as hereinbefore described with reference to any of the examples and/or to any of the accompanying drawings.

Another aspect of the present disclosure is directed to a method for scoring and reporting electroencephalogram (EEG) data for use in transcranial magnetic stimulation (TMS) therapy. The method includes receiving EEG data collected from a subject, removing artifacts from the EEG data, determining a plurality of EEG metrics from the EEG data, and determining a Brain Synchrony Score from the plurality of EEG metrics by applying a predetermined transfer function to the plurality of EEG metrics.

In some embodiments, the method includes reporting, via a user interface, the Brain Synchrony Score. In some embodiments, the Brain Synchrony Score represents a level of synchronous energy in the EEG data that is within an alpha EEG band, the alpha EEG band being approximately 8 Hz to approximately 13 Hz. In some embodiments, the method includes determining at least one of a current frequency, an optimal frequency range, and an interference score based on the EEG data and reporting, via a user interface, at least one of the current frequency, the optimal frequency range, and the interference score. In some embodiments, the current frequency represents a dominant frequency of the subject. In some embodiments, the optimal frequency range represents an optimal frequency range for the subject. In some embodiments, the interference score represents an amount of energy in the EEG data that is within a theta EEG band, the theta EEG band being approximately 5 Hz to approximately 7 Hz.

In some embodiments, the EEG data includes a plurality of EEG channels, wherein each EEG channel corresponds to a scalp electrode used to record the EEG data. In some embodiments, removing artifacts from the EEG data includes filtering out at least one of noise artifacts and eye-blink artifacts. In some embodiments, the EEG metrics include an intrinsic alpha frequency based on a dominant frequency in an alpha EEG band, the alpha EEG band being approximately 8 Hz to approximately 13 Hz. In some embodiments, the predetermined transfer function is generated from at least one factor analysis performed on EEG data collected from a plurality of different subjects. In some embodiments, the EEG data collected from the plurality of different subjects includes EEG data collected before and after TMS treatment. In some embodiments, the at least one factor analysis includes an exploratory factor analysis (EFA) and a confirmatory factor analysis (CFA).

Another aspect of the present disclosure is directed to a system for scoring and reporting electroencephalogram (EEG) data for use in transcranial magnetic stimulation (TMS) therapy. The system includes at least one memory storing computer-executable instructions and at least one processor for executing the instructions stored on the memory. Execution of the instructions programs the at least one processor to perform operations that include receiving EEG data collected from a subject, removing artifacts from the EEG data, determining a plurality of EEG metrics from the EEG data, and determining a Brain Synchrony Score from the plurality of EEG metrics by applying a predetermined transfer function to the plurality of EEG metrics.

In some embodiments, wherein execution of the instructions programs the at least one processor to perform operations that include reporting, via a user interface, the Brain Synchrony Score. In some embodiments, the Brain Synchrony Score represents a level of synchronous energy in the EEG data that is within an alpha EEG band, the alpha EEG band being approximately 8 Hz to approximately 13 Hz. In some embodiments, execution of the instructions programs the at least one processor to perform operations that include determining at least one of a current frequency, an optimal frequency range, and an interference score based on the EEG data and reporting, via a user interface, at least one of the current frequency, the optimal frequency range, and the interference score. In some embodiments, the current frequency represents a dominant frequency of the subject. In some embodiments, the optimal frequency range represents an optimal frequency range for the subject. In some embodiments, the interference score represents an amount of energy in the EEG data that is within a theta EEG band, the theta EEG band being approximately 5 Hz to approximately 7 Hz.

In some embodiments, the EEG data includes a plurality of EEG channels, wherein each EEG channel corresponds to a scalp electrode used to record the EEG data. In some embodiments, removing artifacts from the EEG data includes filtering out at least one of noise artifacts and eye-blink artifacts. In some embodiments, the plurality of EEG metrics include an intrinsic alpha frequency based on a dominant frequency in an alpha EEG band, the alpha EEG band being approximately 8 Hz to approximately 13 Hz. In some embodiments, the predetermined transfer function is generated from at least one factor analysis performed on EEG data collected from a plurality of different subjects. In some embodiments, the EEG data collected from the plurality of different subjects includes EEG data collected before and after TMS treatment. In some embodiments, the at least one factor analysis includes an exploratory factor analysis (EFA) and a confirmatory factor analysis (CFA).

### BRIEF DESCRIPTION OF THE DRAWINGS

The accompanying drawings, which are included to provide a better understanding of the disclosure, illustrate embodiments of the disclosure and together with the description serve to explain the principle of the disclosure. In the drawings:

FIG. 1 is a diagram illustrating a 10-20 system for mapping electroencephalogram (EEG) electrodes in accordance with aspects described herein.

FIG. 2 is a flow chart illustrating a method for scoring and reporting electroencephalogram (EEG) data for use in transcranial magnetic stimulation (TMS) therapy in accordance with aspects described herein.

FIG. 3 is a diagram illustrating a portion of an EEG according to in accordance with aspects described herein.

FIG. 4 is a diagram illustrating alpha bursts in accordance with aspects described herein.

FIG. 5 is a diagram illustrating several Fast Fourier transforms (FFTs) in accordance with aspects described herein.

FIG. 6 is a flow diagram illustrating a process for determining a treatment frequency in accordance with aspects described herein.

FIG. 7 is a diagram illustrating a clinical pipeline in accordance with aspects described herein.

FIG. 8 is a flow diagram illustrating a frequency determination process in accordance with aspects described herein.

FIGS. 9A-9H illustrate a table of example results after a first treatment session in accordance with aspects described herein.

FIGS. 10A-10H illustrate a table of example treatment coefficients after a first treatment session in accordance with aspects described herein.

FIGS. 11A-11I illustrate a table of example results after a full treatment course in accordance with aspects described herein.

FIG. 12 is a diagram illustrating an example super-organizing map fit of training data in accordance with aspects described herein.

FIGS. 13A and 13B illustrate a set of example variable importance plots in accordance with aspects described herein.

FIG. 14 is a diagram illustrating an example fitted conditional inference tree in accordance with aspects described herein.

FIG. 15 is a table illustrating example factor loadings in accordance with aspects described herein.

FIGS. 16A and 16B are tables illustrating an example summary of a weighted least squares estimator in accordance with aspects described herein.

FIG. 17 illustrates example distributions of predicted scores in accordance with aspects described herein.

FIG. 18 illustrates example line plots of several factors and a factor multivariate for a first treatment session in accordance with aspects described herein.

FIG. 19 illustrates example line plots of several factors and a factor multivariate for a full treatment course in accordance with aspects described herein.

FIG. 20 illustrates example tile plots of several factors and a factor multivariate for a single treatment session in accordance with aspects described herein.

FIG. 21 illustrates example tile plots of several factors and a factor multivariate for a full treatment course in accordance with aspects described herein.

FIG. 22 is a table illustrating example factor loadings in accordance with aspects described herein.

FIGS. 23A-23D illustrate a table of an example summary of a weighted least squares estimator in accordance with aspects described herein.

FIG. 24 illustrates an example of a Braincare™ Report in accordance with aspects described herein.

FIG. 25 is a diagram illustrating a quantitative EEG (QEEG) relative power measurement for a normal voltage brain type in accordance with aspects described herein.

FIG. 26 is a diagram illustrating a QEEG relative power measurement for a frontal generator brain type in accordance with aspects described herein.

FIG. 27 is a set of diagrams illustrating a series of QEEG magnitude spectra and QEEG relative power diagrams for a low alpha brain type in accordance with aspects described herein.

FIG. 28 is a set of diagrams illustrating QEEG magnitude spectra and QEEG relative power in accordance with aspects described herein.

FIG. 29 is a set of diagrams illustrating several FFTs and a standardized low-resolution electromagnetic tomographic analysis (sLORETA) for a stroke victim in accordance with aspects described herein.

FIG. 30 is a set of diagrams illustrating a series of QEEG magnitude spectra from EEGs of identical twins with autism in accordance with aspects described herein.

FIG. 31 is a diagram illustrating a series of QEEG magnitude spectra over a period of time for a stroke victim experiencing recovery in accordance with aspects described herein.

FIG. 32 illustrates a series of QEEG relative power diagrams over a period of time for a stroke victim experiencing in accordance with aspects described herein.

FIG. 33 is a set of diagrams that illustrates QEEG magnitude spectra and QEEG relative power for a patient with autism in accordance with aspects described herein.

FIG. 34 is a diagram illustrating a series of QEEG magnitude spectra that shows changes from L-Methylfolate supplementation after identification of an EEG characteristic in accordance with aspects described herein.

### DETAILED DESCRIPTION

The disclosure relates to scoring and reporting electroencephalogram (EEG) data for use in transcranial magnetic stimulation (TMS) therapy.

Brainwaves may be analyzed by considering them as a composite of sinusoidal waves across a spectrum. The spectrum may be divided into various bands. Some of the bands may be known by Greek letter names. Alpha waves may be neural oscillations that occur at about 8-13 Hz. Some other bands in the brainwave spectrum may be: Delta waves (0.5 Hz-3 Hz); Theta waves (3 Hz-8 Hz); Beta waves (13 Hz-38 Hz); and Gamma waves (38 Hz-42 Hz).

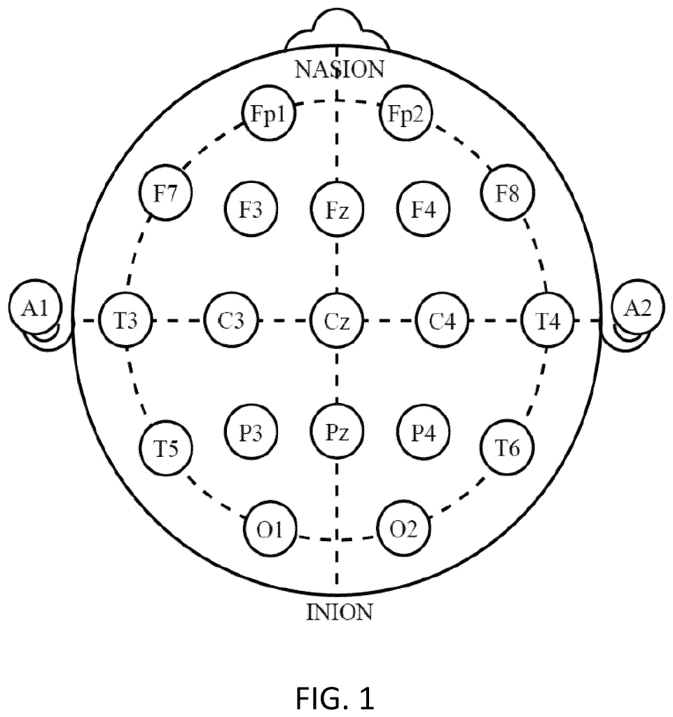

In some examples, referring to FIG. 1 an EEG is recorded according to the 10-20 system. The 10-20 system is an internationally recognized method to describe and apply the location of scalp electrodes in the context of an EEG exam. In some examples, the 10-20 system is based on a relationship between the location of an electrode and the underlying area of the brain. FIG. 1 refers to two anatomical landmarks for positioning of the EEG electrodes. In some examples, the first landmark is a nasion which is the distinctly depressed area between the eyes, just above the bridge of the nose. In some examples, the second landmark is the inion, which is the lowest point of the skull from the back of the head and may be normally indicated by a prominent bump. As depicted in FIG. 1, each electrode placement site has a letter to identify the lobe or area of the brain corresponding to the electrode placement site. For example, some of the letters in the 10-20 system are Fp (pre-frontal), F (frontal), T (temporal), P (parietal), O (occipital), and (C) central. It is to be understood that although there is no “central lobe” of the brain, there may be useful signals detected at the C locations. A “Z” (zero) refers to an electrode placed on the midline sagittal plane of the skull, (e.g., FpZ, Fz, Cz, Oz) and is present mostly for reference or measurement points. In some examples, even-numbered electrodes (2,4,6,8) refer to electrode placement on the right side of the head, whereas odd numbers (1,3,5,7) refer to those on the left. In some examples, the “A” (sometimes referred to as “M” for mastoid process) refers to the prominent bone process usually found just behind the outer ear. FIG. 1 depicts F3, F4, Fz, Cz, C3, C4, O1, O2, A1, A2, T3, T4, T5, T6, T7, P3, P4. In some examples, Cz and Fz refer to ‘ground’ or ‘common’ reference points for all EEG electrodes, and A1-A2 are used for contralateral referencing of all EEG electrodes. It is to be understood that non-contact methods of obtaining EEG signals may use similar nomenclature. It is to be further understood that different quantities and locations for the electrodes may be used, in addition to what is included in the 10-20 system described herein.

Alpha waves are neural oscillations that generally occur at one or more intrinsic frequencies ranging from about 8 Hz to about 13 Hz. They are most evident on an EEG recording when a person is at rest, awake, with eyes closed. It is thought that alpha waves indicate idleness, or a lack of concerted activity in the brain. However, alpha waves are often predominant in individuals who have excellent concentration abilities, are calm, focused, and relaxed. In some cases, alpha activity provides a central role in information processing and control.

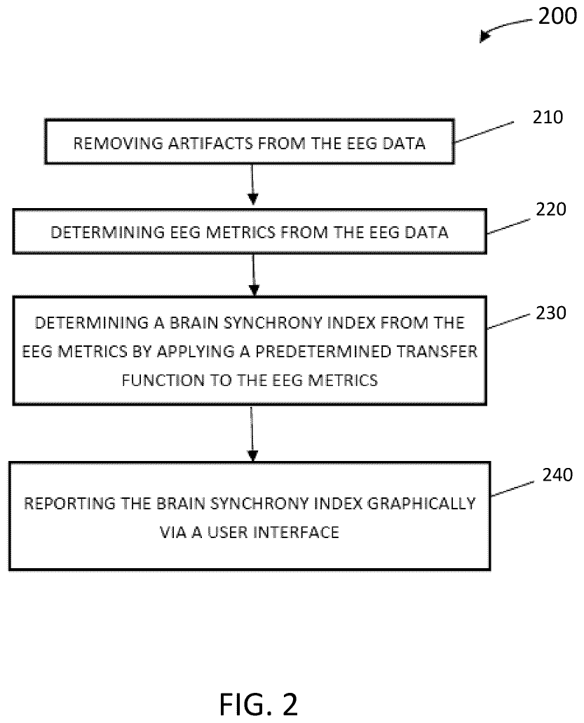

FIG. 2 is a flow chart illustrating a method 200 for scoring and reporting electroencephalogram (EEG) data for use in transcranial magnetic stimulation (TMS) therapy in accordance with aspects described herein The method includes: removing artifacts from the EEG data 210; determining EEG metrics from the EEG data 220; determining a Brain Synchrony Index from the EEG metrics by applying a predetermined transfer function to the EEG metrics 230; and reporting the Brain Synchrony Index graphically 240 (e.g., via a user interface).

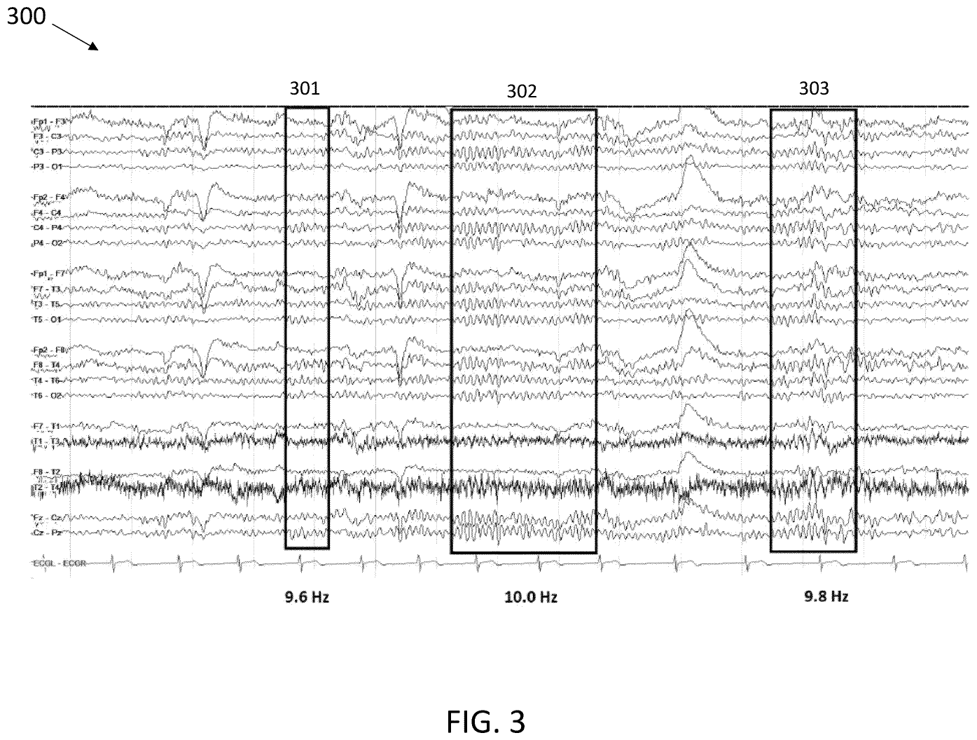

FIG. 3 depicts a portion of an EEG 300 with alpha activity in certain portions in accordance with aspects described herein. The boxes labeled 9.6 Hz (301), 10.0 Hz (302), and 9.8 Hz (303) enclose bursts of alpha activity on the EEG 300. In some examples, the sinusoidal EEG waveforms in the boxes represent alpha activity. Some alpha activity may be identified visually, while other alpha activity may be difficult to identify without signal processing. For example, the alpha activity in the box 302 may be easier to identify than the alpha activity in boxes 301 and 302.

As disclosed herein, brain activity may not be purely random. Some brain activity may occur at a resonant frequency. For the purposes of the disclosure, the brain may be considered a resonant system. The frequencies 9.6 Hz, 10.0 Hz, and 9.8 Hz shown in FIG. 3 are the estimated alpha frequencies for the bursts enclosed by each box. In some examples, alpha bursts are considered separately, or in aggregates following various rules. In some examples, an average alpha frequency is calculated for a quantity of alpha bursts. As depicted in FIG. 3, the average alpha frequency may be about 9.8 Hz for the entire EEG 300. In some examples, an EEG has regions with minimal to no alpha activity, or there may be strings of alpha activity bursts.

As disclosed herein, an optimal frequency for TMS may be different for each individual. In some examples, an optimal TMS frequency is determined, at least in part, by analyzing the EEG of the brain. In some examples, one step in determining the optimal TMS frequency is to identify and label the alpha bursts. In some examples, alpha bursts are identified by wavelet correlation (e.g., by applying a Morlet wavelet). In some examples, the Morlet wavelet is correlated along the entire EEG waveform. In some examples, a threshold is used to indicate alpha bursts. Burst identification determines when most of the energy of the signal is at a target frequency rather than a more chaotic mixture of frequencies. In some examples, alpha bursts are identified, frequencies of the bursts are determined, and an average frequency is determined for the recorded EEG. In some examples, the optimal TMS frequency is the average frequency for the recorded EEG.

In some examples, a plurality of options exist for determining the optimal TMS frequency. For example, an average frequency may be determined for alpha bursts from various sets of channels on the EEG. In some examples, each individual burst is collected. In some examples, bursts are collected from a specific place, such as where the burst is occurring or a part of the brain where the burst is coming from. In some examples, a histogram of bursts is built from a collection of burst data. In some examples, burst amplitude is determined from the bursts as an indicator of the power of each burst. In some examples, a dual threshold is applied. As used herein, a dual threshold is defined as a process of determining a standard deviation of an average burst amplitude. For example, a burst that is high may be one or greater standard deviations above the average, a burst that is low may be one or fewer standard deviations below the average, and a burst that is normal in amplitude may be near the average.

In some examples, an optimal TMS frequency is determined based on all the previously mentioned information about burst averages and amplitude along with the burst frequency. In some examples, the information about burst averages, amplitudes, and/or frequencies contributes to a determination of desirable and/or undesirable frequencies for TMS. In some examples, an optimal frequency matches a real alpha burst. In some examples, a stimulation frequency corresponding to the average frequency is not be considered an optimal TMS frequency unless the average frequency coincides with an activity already existing in the brain to which the optimal TMS frequency is applied. In some examples, the stimulation frequency reflects an activity already existing in the EEG of the brain to which the optimal TMS frequency is applied.

Examples of systems and methods for providing TMS and treatments based on EEG data can be found in PCT Publication No. WO/2022/204725, filed on Mar. 25, 2022 and titled “ELECTROENCEPHALOGRAM (EEG) BASED TRANSCRANIAL MAGNETIC STIMULATION (TMS) DEVICES,” PCT Publication No. WO/2022/204726, filed on Mar. 25, 2022 and titled “ELECTROENCEPHALOGRAM (EEG) BASED TRANSCRANIAL MAGNETIC STIMULATION (TMS) DEVICES,” and PCT Publication No. WO/2022/204727, filed on Mar. 25, 2022 and titled “ELECTROENCEPHALOGRAM (EEG) BASED TRANSCRANIAL MAGNETIC STIMULATION (TMS) DEVICES,” each of which is hereby incorporated by reference in its entirety.

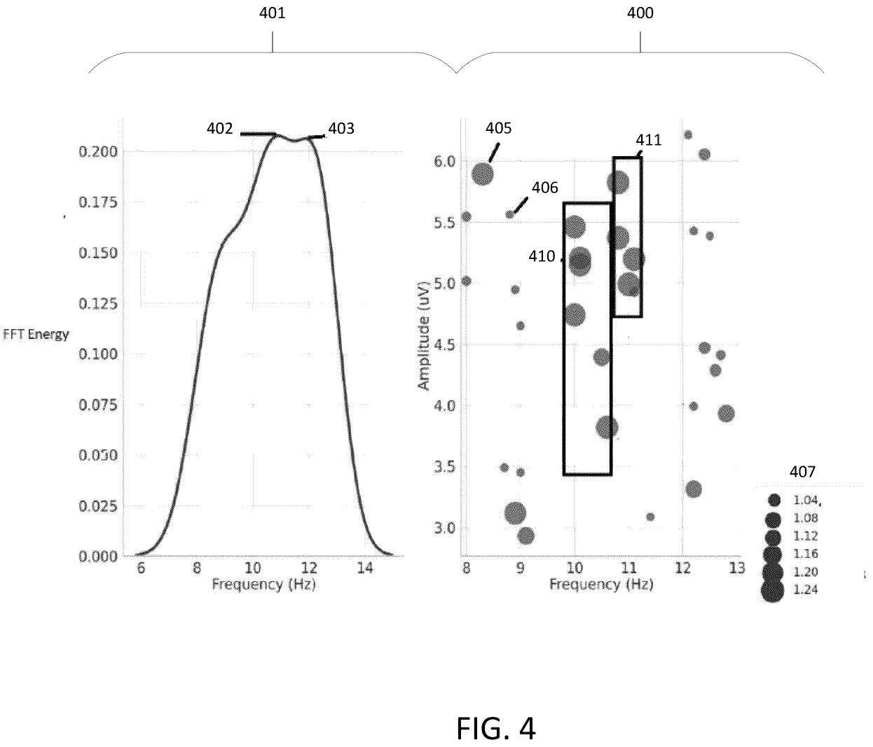

As shown in plot 400 of FIG. 4, circles may be used to represent alpha bursts (e.g., 405, 406). In some examples, the diameter of each circle is indicative of a duration of the burst (e.g., as indicated in key 407). In some examples, an average amplitude of the burst represented by a circle is indicated by a position along a Y axis. In some examples, increased alpha activity is shown higher up on the Y axis. Likewise, if the alpha activity of a burst is rather low, then the burst may be more chaotic. In the time domain, it may be relatively difficult to visually identify an alpha waveform of a low activity alpha burst.

In FIG. 4, boxes 410, 411 each indicate a dominant frequency. The boxes 410, 411 group bursts of similar frequency, although there may be different amplitudes and different lengths for the bursts. Plot 401 on the left is a kernel density estimate (KDE) plot, which depicts a more typical frequency spectrum diagram based on the same data as plot 400 on the right. In some examples, the peaks 402, 403 represent higher energy activity. In some examples, peak 402 corresponds to box 410 and peak 403 corresponds to box 411. As shown, the same data may be represented in two different ways. In some examples, the plot 400 on the right provides a sense of the “bursty” activity.

In some examples, plot 400 and/or plot 401 of FIG. 4 are included in a report that is provided to a clinician to describe to a patient or a client details regarding the patient's brain activity. It has been determined that healthier brains tend to have more rhythmic activity. In healthier brains, the bursts tend to be more frequent, the bursts tend to last longer, and the alpha tends to be more significant and easier to visualize. It is to be understood that the frequencies may vary across a spectrum for a human population. As disclosed herein, some mental disorders may be associated with significantly little alpha activity. Referring to FIG. 4, more large circles, especially higher amplitude, large circles, may be indicative of a relatively healthy brain.

In some examples, the process for determining alpha frequencies includes artifacting. As used herein, artifacting is defined as filtering noise artifacts from data. In some examples, burst activity is determined from the individual channels (e.g., EEG channels). In some examples, certain channels give less usable data. In some examples, channels where electrodes are close to the eyes have less usable data because the electrodes that are close to the eyes have more eye-blink artifacts than channels where electrodes are farther back on the scalp. If there is a significant, easily detectable artifact, the data for the interval of time that contains the artifact is filtered out for all of the channels during that interval, or the data from the particular channel in which the artifact was observed is filtered individually. In some examples, for determining alpha, preference is given to posterior channels. In some examples, a weighting schema is used to give preference to certain channels for the determination of alpha.

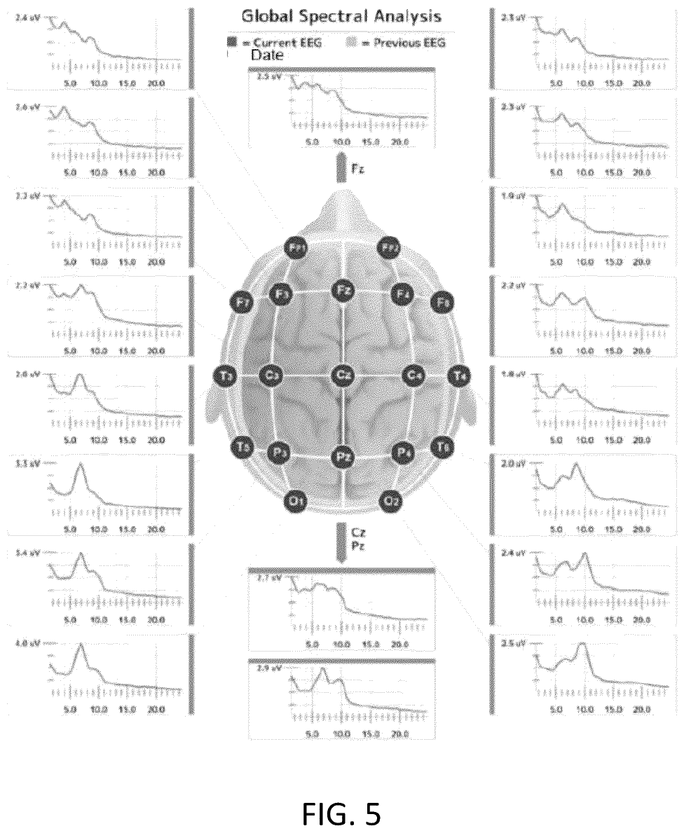

In some examples, multiple (e.g., three or four) channels are processed together with an FFT based off of the aggregate of the channels from a region. In some examples, regional clustering is based on posterior, central, or frontal channels. In some examples, an FFT is calculated for each channel individually, and a report includes a plurality of FFTs across the subject's head, as shown in FIG. 5.

In some examples, channels are processed individually or in clusters of any set of channels. In some examples, a report includes a visualization to see a frontal cluster and a posterior cluster. In some examples, posterior changes are observed before frontal changes. It is to be understood that any set of channels may be combined. In some examples, every channel is considered, and a subset of the channels that were considered are presented in a report.

In some examples, a moving window is used to step through an EEG channel. Processing of time windows of the EEG channel or EEG channels may include an overlap, or there may be no overlap of the time windows. In some examples, the time windows have a duration of about one second, or about one half second. In some examples, the time windows have variable durations. For example, a first window may have about one quarter second duration and the next window may have about one half second duration. In some examples, each window of the moving window is analyzed and/or scored to determine whether or not the window includes a burst.

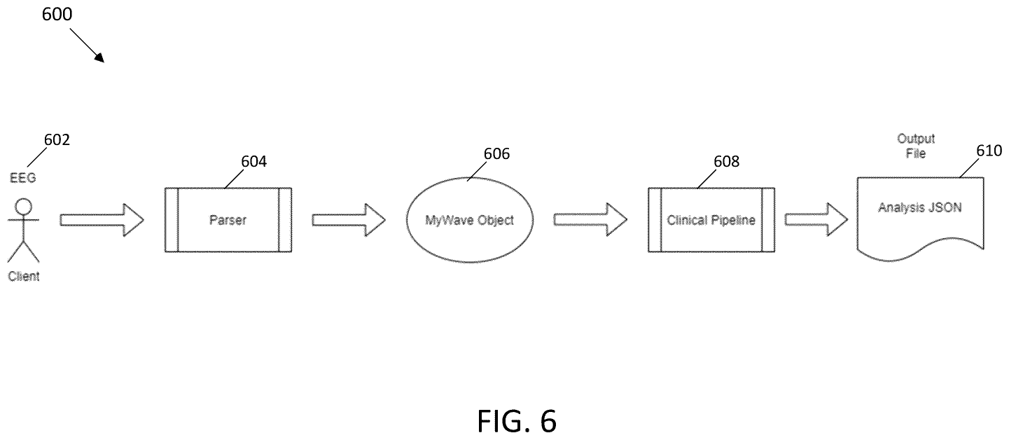

FIG. 6 illustrates a process 600 for determining a treatment frequency. In some examples, the process 600 is referred to as the “MyWave Analytics workflow”. As shown, the process 600 includes several pieces. In some examples, an EEG 602 is provided through a parser 604 to transform the EEG 602 into a MyWave object 606. In some examples, the MyWave object 606 wraps an MNE[1] object. After the EEG 602 successfully parsed, the MyWave object 606 goes through a clinical pipeline 608. In some examples, the clinical pipeline 608 includes a series of steps to be performed on the MyWave object 606 (e.g., the parsed EEG 602). In some examples, at the end of the clinical pipeline 608, an analysis file 610 is created with the results. In some examples, the analysis file 610 is provided in a JSON format.

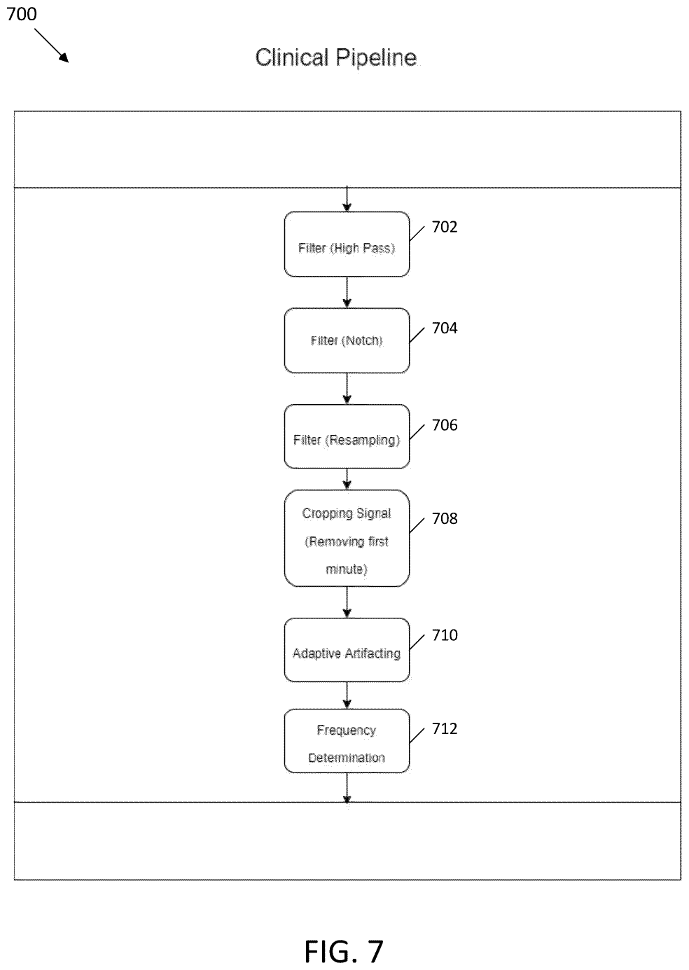

FIG. 7 illustrates a clinical pipeline 700. In some examples, the clinical pipeline 700 corresponds to the clinical pipeline 608 of process 600 of FIG. 6. In some examples, the clinical pipeline 700 includes a first filter 702. In some examples, the first filter 702 is a high pass filter (e.g., ˜0.1 Hz) to remove signal drift. In some examples, the clinical pipeline 700 includes a second filter 704 that follows the output of the first filter 702. In some examples, the second filter 704 is a notch filter configured to remove line noise. In some examples, the clinical pipeline 700 includes a third filter 706 that follows the output of the second filter 704. In some examples, the third filter 706 is a resampling filter. In some examples, the clinical pipeline 700 includes a cropping module 708 that follows the output of the third filter 706. In some examples, the cropping module 708 is configured to crop a leading portion of the EEG signal (e.g., about the first minute of the signal). In some examples, the clinical pipeline 700 includes an adaptive artifacting module 710 that provides one or more artifacting functions. In some examples, the adaptive artifacting module 710 follows the output of the cropping module 708. In some examples, the adaptive artifacting module 710 includes two or more sub-modules. In some examples, the clinical pipeline 700 includes a frequency determination module 712 that determines the EEG frequency (or frequencies). In some examples, the frequency determination module 712 follows the output of the adaptive artifacting module 710.

In some examples. the adaptive artifacting module 710 uses an iterative process to determine thresholds to ensure a portion (e.g., at least 80%) of the data meeting selection criteria is retained while eliminating the data that does not meet selection criteria (e.g., the remaining 20%). In some examples, the adaptive artifacting module 710 include three parts (or sub-modules): a gross movement (e.g., muscle/electromyography (EMG)) artifact detector, a blink artifact detector, and an iterative loop. In some examples, the EEG signal is annotated and time segments with artifacts are removed. In some examples, no independent component analysis (ICA) or signal repair takes place.

In some examples, the gross movement artifact detector detects low frequency (e.g., ˜0.5-˜1.5 Hz) power above a certain threshold. In some example, gross movement is detected by a comparing the low frequency power to a changing iterative threshold. In some examples, the blink artifact detector may detect low frequency (e.g., ˜1-˜3 Hz) power that has a power spike over a certain threshold that occurs within a short timespan (e.g., ˜0.5 s). In some examples, after the adaptive artifacting module 710 is run, the total amount of data that was eliminated is observed. In some examples, if the amount of data that was eliminated is greater than ˜20%, the thresholds are increased so that more data may be retained. However, there may be an upper limit. In some examples, if thresholds are increased (e.g., gradually from ˜0.4 to ˜1 in increments of ˜0.1) and ˜80% of data still cannot be retained, then a flag may be triggered to indicate that the EEG has failed the artifacting process. In some examples, a complex decision tree is used to determine a frequency of treatment. In some examples, there are five potential outcomes, based on how well a variety of algorithms perform. In some examples, the frequencies selected are bound between ˜8 and ˜13 Hz.

In some examples, power spectrum analysis is done using a Fourier transform to convert the signal into the frequency domain. In some examples, if a valid peak is found (e.g., peak validity may be determined by the height compared to the surrounding data), then the power spectrum analysis is considered successful. In cases where there are multiple valid peaks, a Parzen[2] weighting window is applied before searching for the largest amplitude peak. In some examples, the weighting window weighs peaks towards the center of the range (e.g., ˜10.5 Hz) more heavily than peaks near the border of this range. In some examples, burst analysis is an algorithm utilized to provide information even in cases where a power spectrum analysis fails. The burst analysis algorithm includes two parts. One part determines when the bursts are occurring. Another part determines the dominant frequencies of those bursts. In some examples, the data is aggressively band-passed to pass power in the alpha range. After band-pass filtering, the data is normalized to the median power so that a dual threshold approach may be applied to determine when a burst occurs. In some examples, a burst occurs when the power climbs above an upper threshold. The burst is considered to remain in the bursting state until the power dips below a lower threshold.

After the algorithm determines when a burst occurs, a series of narrow bandpass filters are applied in order to determine a dominant frequency for each burst. As used herein, a dominant frequency is defined as a frequency having a highest power response in a frequency band. In some examples, the frequency is determined from burst analysis alone. In such cases, another algorithm is applied and a confidence level determined. In some examples, the frequency is the frequency bin (e.g., 0.1 Hz) that contains the most bursts. Confidence is determined from various factors that may include: number of bursts detected, relative alpha power, amount of artifact in the EEG, or outcome of power spectrum analysis. Some examples of confidence levels are: “No confidence”, “low confidence”, “medium confidence”, and “high confidence”.

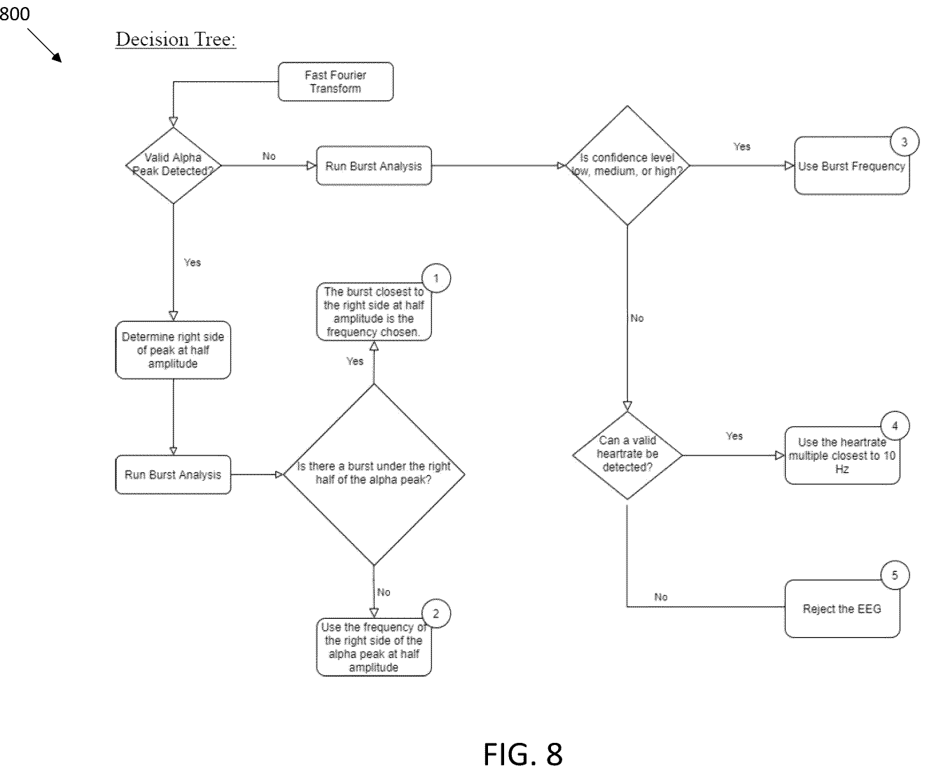

FIG. 8 illustrates a flow diagram 800 representing the frequency determination process. As shown, there may be five different outcomes for frequency determination. One of the outcomes results in the rejection of the EEG. In some examples, the different outcomes are based on the performance of power spectrum and burst analyses. A successful power spectrum analysis results in outcomes 1 and 2 shown in the flow diagram 800. Likewise, an unsuccessful power spectrum analysis results in outcomes 3-5.

In some examples, a report is created, and then a treatment protocol is generated based on the report. In some examples, the treatment protocol specifically identifies a treatment frequency for delivery via magnetic pulses. In some examples, the patient undergoes the treatment over a period of about four to about six weeks, about every weekday getting the stimulation pulses at the treatment frequency.

In some examples, a patient's brain is modeled as a resonant system. For example, a pendulum or a playground swing may be another type of resonant system. The pulses delivered via TMS may be similar to light pushes on the playground swing. In some examples, magnetic pulses are delivered at an effective frequency to cause resonance without requiring a large input of energy. In some examples, the pulses are sub-threshold. In some examples, the pulses do not cause any of the neurons directly to fire. In some examples, the magnetic pulses create an electric field around the neurons to encourage the neurons to fire at the pulse frequency. In some examples, the magnetic pulses lightly influence the neurons. In some examples, because it is a resonant system, and the brain has entrainment effects, the brain becomes more rhythmic. During stimulation, there is a time period of hyper synchronous activity in which the brain activity aligns along the resonant frequency. When the stimulation is stopped, due to the plasticity of the brain, the brain may partially revert to the way the brain was operating before the stimulation. However, after the stimulation is repeated over multiple sessions, the brain reverts less and the effect of the stimulation has more permanence.

When the brain becomes relatively more rhythmic, regular, and coherent, symptoms of a mental disorder may change. In some examples, symptoms of mental disorders are excluded from decision making. In some examples, a goal is to make the brain relatively more rhythmic, regular, and coherent. When the brain becomes relatively more rhythmic, regular, and coherent, then the symptoms of mental disorders tend to be reduced. Examples of some mental disorders that may improve with a more rhythmic, regular, and coherent brain may include autism spectrum disorder, Alzheimer's disease, Attention Deficit Hyperactivity Disorder (ADHD), schizophrenia, anxiety, depression, coma, Parkinson's disease, substance abuse, bipolar disorder, sleep disorder, eating disorder, tinnitus, traumatic brain injury, post-traumatic stress disorder, or fibromyalgia.

In some examples, treating a healthy individual and making the healthy brain relatively more rhythmic tends to improve performance in several areas. For example, improvements may be noticeable in mental focus and concentration, performance, and/or sleep quality. There may be many benefits to a relatively more rhythmic, regular brain than a chaotic, high-energy brain, even among a population with healthy brains.

Some different structures of the brain may be overactive or underactive. An overactive brain, from a perspective of brain wave activity, may have a relatively more chaotic EEG. An overactive brain may have less synchronous, bursty, oscillatory activity. In some examples, the processes of the disclosure calm the overactive brain in synchronized function, thereby converting overactive brain tissue to a more normalized state, relative to being overactive.

An underactive brain may have relatively more rhythmic activity. In such a situation, TMS may be used to de-synchronize the brain, which may activate that tissue function. As used herein, a “double hump EEG” is defined as an EEG with multiple dominant frequencies in the alpha band. It may be as though there are competing frequencies between different regions such that the neurological communication may be like a radio transmitter and receiver that may be tuned to different frequencies. The communication between regions of the brain that operate at different frequencies may not be as effective as it would be if the regions were operating at the same frequency.

In some examples, one of the two competing frequencies is chosen for stimulation. By stimulating at that chosen frequency, the regions begin to resonate at the same frequency. In some examples, the lower frequency is pulled up to match the higher frequency. In some examples, the stimulating frequency is between the measured frequencies.

In some examples, the choice of stimulation frequency depends, in part, on the age of the individual. At different ages, there may be an expected frequency or a range of frequencies. In some examples, the dominant frequency is a metric of brain health.

The typical frequency may be referred to as “speed” herein and may change with age. Young people (e.g., about 2-5 years old) may typically have a slower speed. People may tend to speed up to a critical fastest frequency at about age 10 to about age 13. Typically, people may slow down from about age 11 (about 10 Hz-about 11 Hz) to about age 70 (about 9 Hz-about 10 Hz). A typical 70-year-old with an 11 Hz brain may tend to have better working memory than a 20-year-old with a 9 Hz brain. Brain speed may relate to working memory and function.

Examples of speed selection: A 30-year-old who has a peak at about 8.5 Hz and another peak at about 11 Hz may normally have a TMS at about 11 according to some of the example processes disclosed herein. In the example of the 30-year-old, the average of about 8.5 Hz and about 11 Hz may not be selected. A 70-year-old, who has a peak at about 8 Hz and another peak at about 10 Hz may have TMS at about 10 Hz to target the best function for age 70. The selected target frequency may be about 10 Hz if the EEG had enough bursts at about 10 Hz such that the brain structure may appear capable of accommodating the selected target frequency.

If a person has a peak at about 10 Hz, and another peak at about 13 Hz or about 14 Hz, the faster speed may be considered too fast for alpha, and a speed that may be unsustainable. Therefore, applying the processes of the disclosure, the target may not be about 13 Hz to about 14 Hz. In this example, rather than choosing the fastest speed, the target may be between about 10 Hz and about 13 Hz. The age of the person may be considered among other factors.

In some examples, a natural resonance of the brain is advantageously considered. An average stimulation frequency may normally not be at a resonant frequency of the brain. In some examples, the brain has two resonant frequencies. If the natural resonance of the brain is normally at about 10 Hz, stimulating at a higher frequency may not move the natural frequency of the brain to about 10.5 Hz or about 11 Hz. As disclosed herein, the brain may be limited in the ability to shift frequencies. In some examples, an objective of TMS is to make the brain more rhythmic, and, if possible, target a faster frequency that the brain is capable of attaining. For example, if an EEG has a resonance at about 9.5 Hz, and nothing between about 10 Hz and about 11 Hz, the brain may not be expected to accommodate about 10 Hz to about 11 Hz as a target. The brain may have been structurally capable of higher speeds in the past, but as the brain ages, the brain structure may change. It is the current brain structure that may accommodate the function disclosed herein. In some examples, if TMS is delivered at a frequency that the brain structurally cannot accommodate, the brain reverts to the previous frequency immediately upon ending the TMS session.

Some clinicians recommend providing a TMS application of about 10 Hz. For example, everyone presenting with depression may be treated with about 10 Hz TMS according to some versions of standard TMS practice. The clinical outcome may be linearly correlated to the distance of the patient's Intrinsic Alpha Frequency (IAF) versus the treatment frequency. The further the treatment frequency is away from a rhythm within the operational capability of the brain, the worse the clinical outcome may be.

IAF represents the dominant brainwave frequency in the alpha EEG band (e.g., about 8 Hz to about 13 Hz). In some examples, IAF varies between individuals, with each person evincing a different dominant alpha frequency. In some examples, this dominant alpha frequency tends to vary over the long term, with IAF dropping approximately ¼ Hz per decade after age 50. Low alpha frequency may often be associated with cognitive problems and mental disorders. For example, dementia and Alzheimer's disease patients tend to have alpha rhythm well below about 8.0 Hz. It is often seen that cognitive difficulty occurs when a person's IAF drops below about 8.0 Hz.

In some examples, IAF may vary for a person over the short term. This intra-subject Alpha Frequency Variability (AFV) is a result of different alpha networks kicking-in in response to task demands. In some examples, AFV reflects fluctuations in moment-to-moment performance, and it changes based on the person's mental state. AFV may be one reason for the large bandwidth seen in spectral analysis of the EEG waveform as a whole. However, IAF may be clearer and show less variability when the EEG is analyzed in small temporal increments. Alpha activity may tend to be bursty, with short duration Alpha bursts or “Alpha spindles” evident. These Alpha bursts may last from about 0.5 sec to about 2.0 sec in duration or longer. Some individuals may spend the majority of their waking hours in an Alpha burst state. Others may have no discernable Alpha burst at all.

In some examples, the intrinsic frequency of other EEG bands also exhibits variability between bursts. For example, when a person transitions to certain stages of sleep, sleep spindles may often be visible on an EEG recording, with an intrinsic frequency between about 11 Hz and about 16 Hz. These may be thought of as Beta bursts. Other intrinsic frequencies of other EEG bands may exhibit similar burst-like behavior.

As disclosed herein, the IAF recorded during individual Alpha spindles may vary from burst to burst. A slight variability (e.g., up to about 0.2 Hz) may be common. However, significant variability (e.g., above about 0.25 Hz) may be associated with poor cognitive processing or a potential mental disorder. Therefore, the optimal brain state or brain health may be such that the variability in IAF from one Alpha burst to another may be minimized.

As disclosed herein, the prevalence of Alpha activity may be indicative of better mental health. A person who is calm, relaxed, and able to focus on single tasks may often have Alpha bursts that are longer and have higher amplitude than someone who is less able to focus or who may suffer from stress or anxiety. In addition to AFV, there may be duration and time interval between Alpha bursts that provide an indication (e.g., from an indicator) of brain state or brain health. In general, a brain's mental state and health may be better when a greater percentage of time is spent in the Alpha burst. This may be referred to as Alpha Prevalence (AP).

In some examples, brain stimulation at a stimulation pulse frequency that is equal to an intrinsic frequency of an EEG band (e.g., IAF) has an effect on symptoms of major depression and other disorders. Due to the brain's natural variability in alpha frequency, the optimum stimulation frequency may be imprecisely defined. In some examples, the value is different depending on the time and duration of a recording. To overcome this limitation, other brain stimulation processes use a stimulation frequency set to an intrinsic EEG frequency that has been determined over an extended period of time and which allows for natural variability of IAF to be averaged into one overall estimate.

In some examples, a best frequency to choose for TMS is determined by applying a rule set to the EEG data. For example, a particular rule set may be applied for Post-Traumatic Stress Disorder (PTSD). Another rule set may be applied for patients presenting with autism. Yet another rule set may be applied for patients presenting with Mild Cognitive Impairment (MCI). All of these rule sets may be implemented automatically in a computer system (e.g., as a computer program).

In some examples, a Brain Synchrony Index is determined from EEG metrics by applying a predetermined transfer function to the EEG metrics. In some examples, the transfer function is based on an analysis of a plurality of EEGs from different people who have gone through TMS (e.g., a minimum of 30,000 people). In some examples, the EEGs may be linked to each person in a database. In some examples, clinical scores are linked to each patient and stored in the database. Likewise, the data may be stored in a database for analysis. In some examples, EEGs are stored before and after TMS. In some examples, each individual EEG is cleaned with automated artifact rejection. Treatment may be recommended for a specific time period, e.g., about four to about six weeks of treatment. Various EEG metrics may be determined from the data. Examples of EEG metrics include coherence, amplitude, and burst statistics. In some examples, the EEG metrics are stored in a database.

In some examples, the transfer function is determined, in part, by performing an exploratory factor analysis (EFA). The EFA is used to determine which factors were responsible for the highest percentage variants between the pre-TMS EEG data and the post-TMS EEG data. Some factors of the EFA include relative theta, relative alpha power, coherence between specific channels, and a measurement that specifies a synchrony of an FFT calculated from an EEG. In some examples, about half of the data in the database is used for EFA, and the other half is used for a Confirmatory Factor Analysis (CFA).

In some examples, a super-organizing map (SOM) is fit on the EEG training data using a hexagonal toroidal grid. Map quality is assessed using count, quality, and neighbor distance plots. Codebook vectors are bound into a single data frame and an optimal number of clusters are identified using silhouette and gap statistic methods and clustering using medoids. To obtain a set of variables relevant for defining clusters, (1) the codebook vectors for each cluster are examined, (2) a random forest is fit to classify the data to the SOM-generated clusters and the importance plot is examined, and (3) a conditional inference tree is fit. Based on these measures of importance, a subset of variables is selected for use in the EFA. Specifically, all variables used in the conditional inference tree are collected, along with the top (e.g., 5) important variables used to construct the random forest as identified by mean decrease in accuracy and mean decrease in Gini (e.g., 10 variables total), and the top (e.g., 5) variables with high median codebook vectors in each cluster are added to this set. Unique variables in the set are retained and any factor variables are removed. The result is the final set of important variables used for EFA.

In some examples, the first EEG for each patient is extracted and the data set is randomly divided 50/50 into a training set for EFA and an independent test set for CFA. The important variables identified are selected. The training set is assessed for normality and transformations to achieve normality are attempted via Box-Cox and standard transformations (e.g., log, logit, cube root, etc.). A correlation matrix is inspected visually and using the Kaiser, Meyer, and Olkin (KMO) Measure of Sampling Adequacy (MSA). Highly correlated variables are removed until a KMO>0.8 is achieved and variable correlations are moderate. The number of factors are estimated using parallel analysis. EFA is then performed using the number of factors determined by parallel analysis and varimax rotation. Factor scores are output for interpretation using an R package (e.g., readxl). The variable with the highest loading on a factor is assigned to that factor. A CFA model is written to reflect the results of the EFA.

In some examples, the CFA model is fit to the data using an R package (e.g., lavaan) with the latent variable variance constrained to 1 for the model specified by the EFA. Variables are scaled as needed to keep variances constant. The CFA is assessed for adequacy using the root mean square error of approximation (RMSEA), Comparative Fit Index (CFI), and Tucker-Lewis Index (TLI). Factor scores are predicted on the full data set for each patient that had non-NA values for the variables in the CFA model. A multivariate score is generated by taking the average of the factor scores.

In some examples, the process for EFA/CFA includes a combination of processes. In some examples, temporal models and/or tests are used to determine patient improvement in individual variables. In some examples, such temporal models and/or tests are applied to patient data collected following a first treatment session and/or a full treatment course. In some examples, feature extraction is performed using an iterative series of EFA/CFA analyses. Exemplary processes and analyses are outlined in the following examples:

Simple Temporal Models and Tests

Introduction: This analysis provides results of simple temporal models and tests to accompany more detailed temporal modeling.

Statistical Methodology: All analyses were performed using the statistical programming language R (R Core Team 2020). For each variable of interest for the first session, the change between the final and baseline score was computed (final-baseline). The mean, median, and standard deviation of the change were computed, and if normality assumptions were satisfied, a one-sample t-test was performed. Otherwise, a one-sample Wilcoxon test was performed. The alternative hypothesis was that the true mean (t-test) or location (Wilcoxon test) was not equal to 0. Hence a p-value<0.05 provides support for a non-zero change in the variable over the course of the first session. p-values were adjusted for multiple comparisons using the Benjamini & Hochberg False Discovery Rate (FDR) method. Since the duration of the session was controlled, and the days of treatment were correlated with the number of treatments, a rank-based regression was fit in the R package Rfit with Change as the response and explanatory variables Treatments (cumulative cortical counts), Age, Gender, and the interaction between Age and Gender. This asymptotically distribution-free rank-based procedure for testing hypotheses using the Jaeckel-Hettmansperger-McKean test statistic assumes a multiple regression setting and a symmetric error distribution. The coefficient estimate for treatments as well as the p-value were retained. The aim was to test the hypothesis that the coefficient estimate for treatments was not equal to 0 for all variables of interest. Hence, the retained p-values were adjusted for multiple comparisons using the FDR method. For each variable of interest for the full treatment data set, the change between the final and baseline score was computed (final-baseline). The mean, median, and standard deviation of the change were computed, and if normality assumptions were satisfied, a one-sample t-test was performed. Otherwise, a one-sample Wilcoxon test was performed. The alternative hypothesis test was that the true mean (t-test) or location (Wilcoxon test) was not equal to 0. Hence a p-value<0.05 provides support for a non-zero change in the variable over the course of the patient's full treatment history, which may involve more than one session. p-values were adjusted for multiple comparisons using the FDR method.

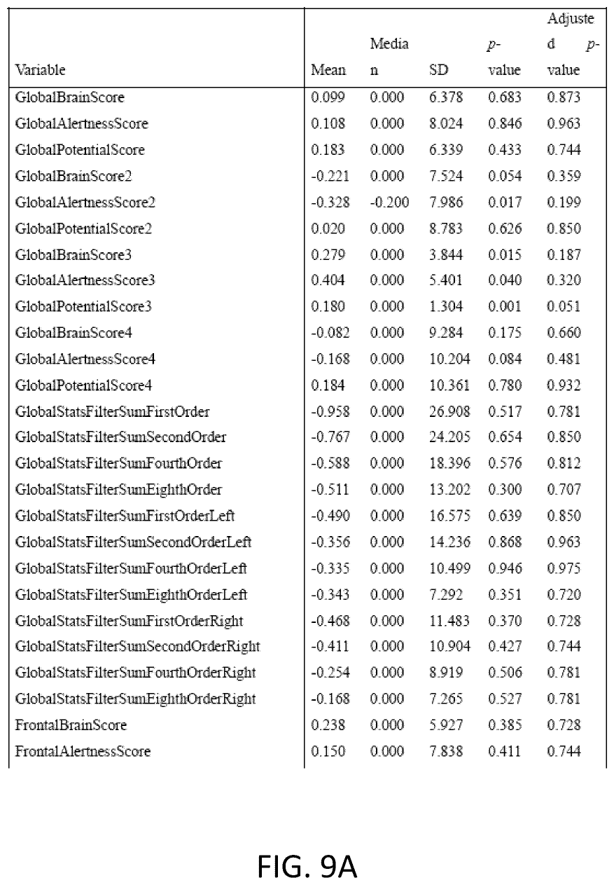

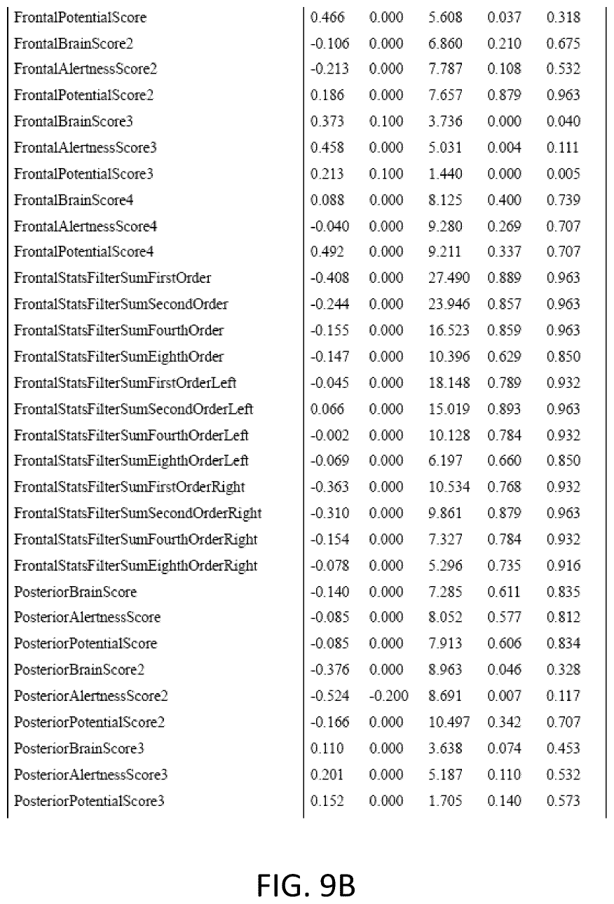

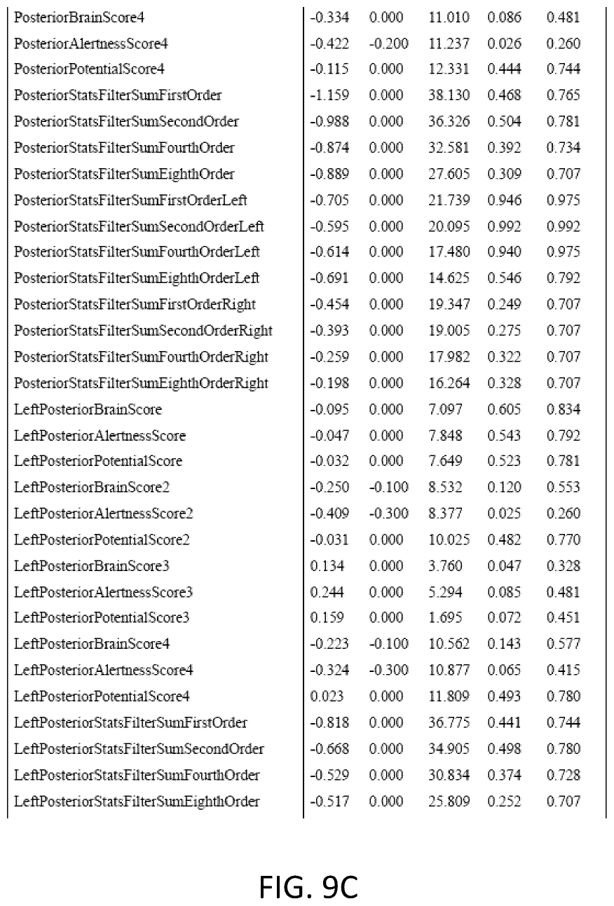

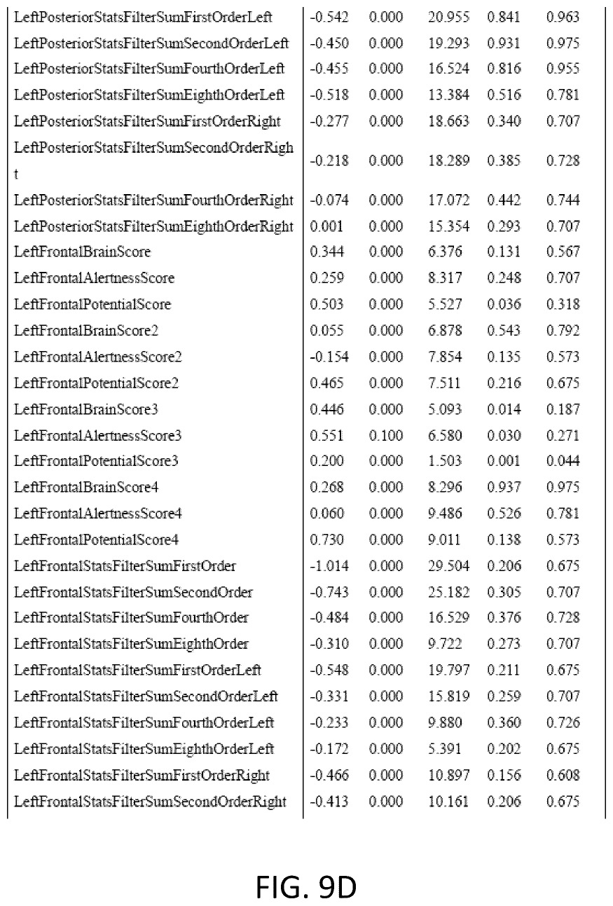

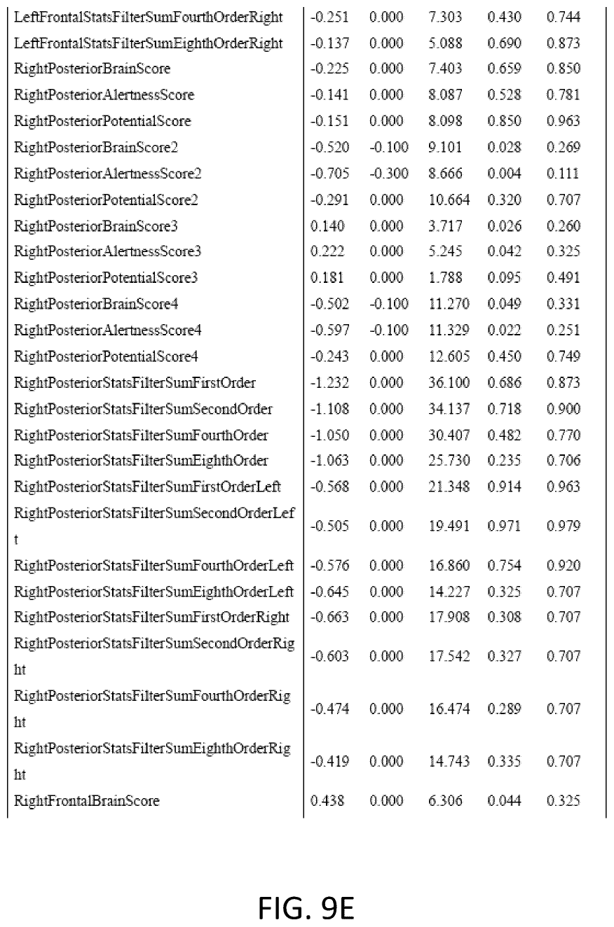

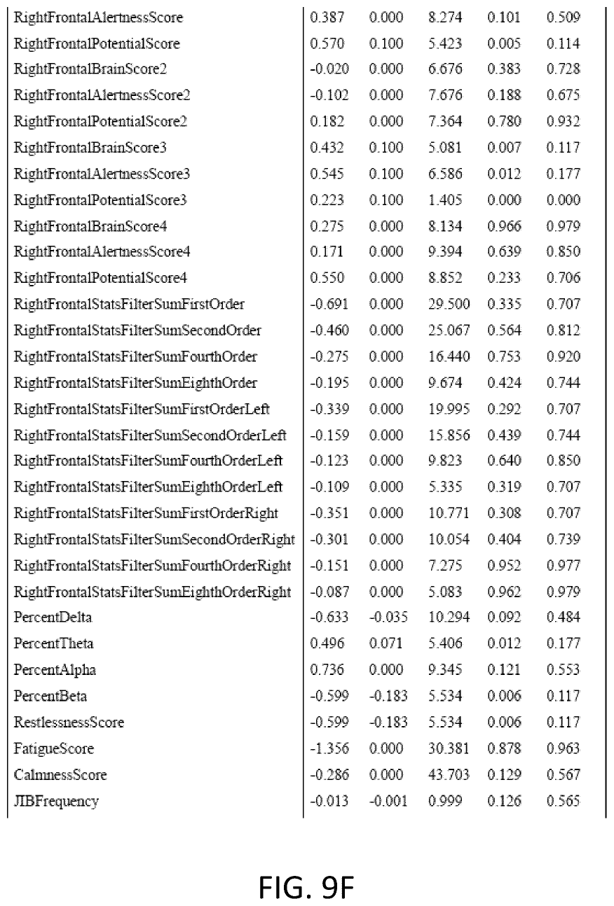

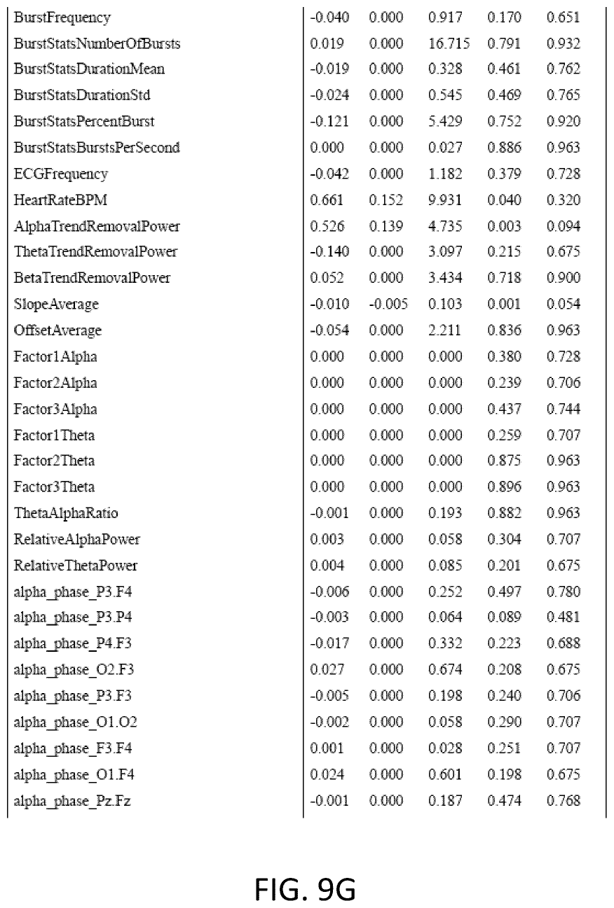

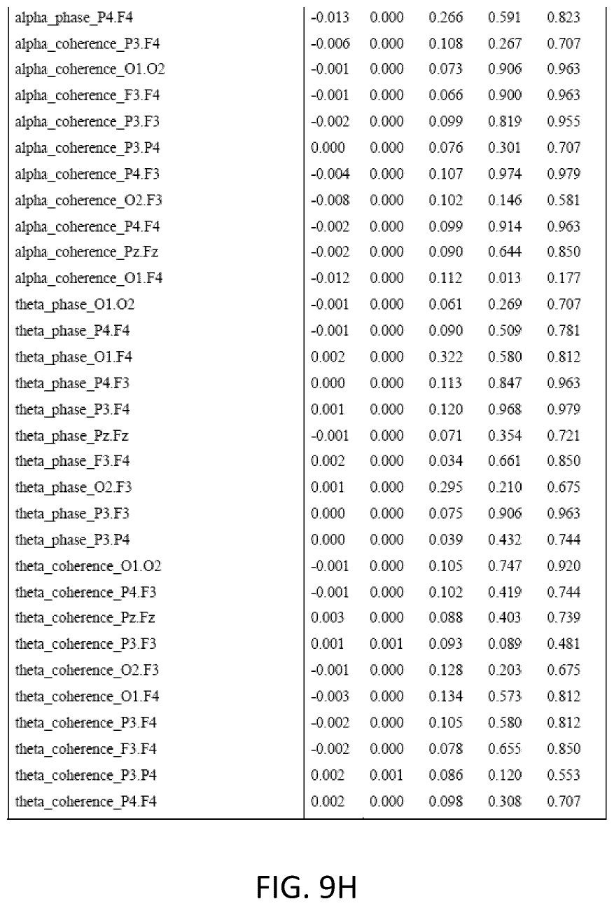

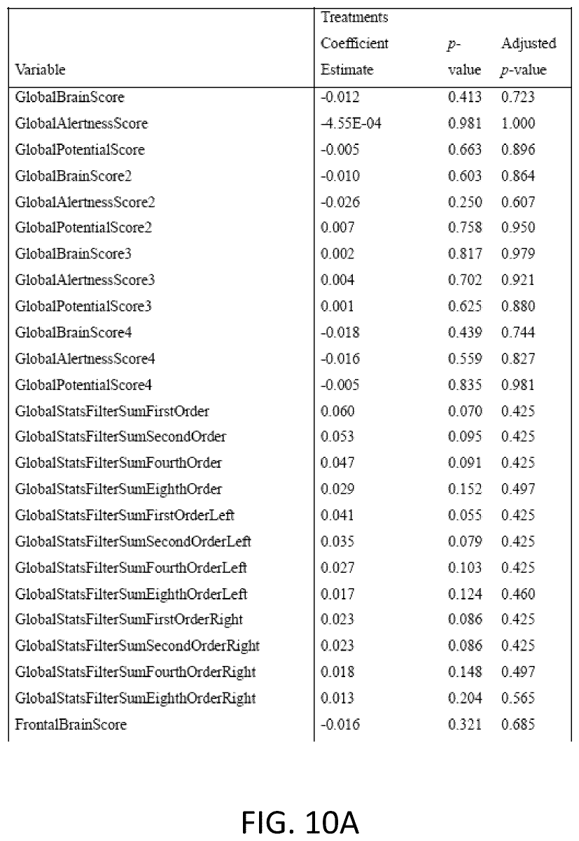

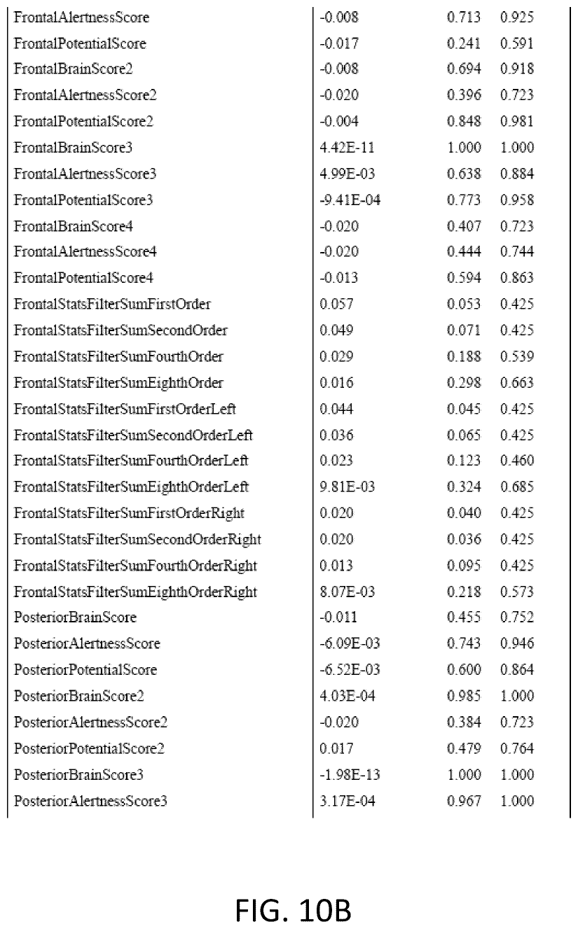

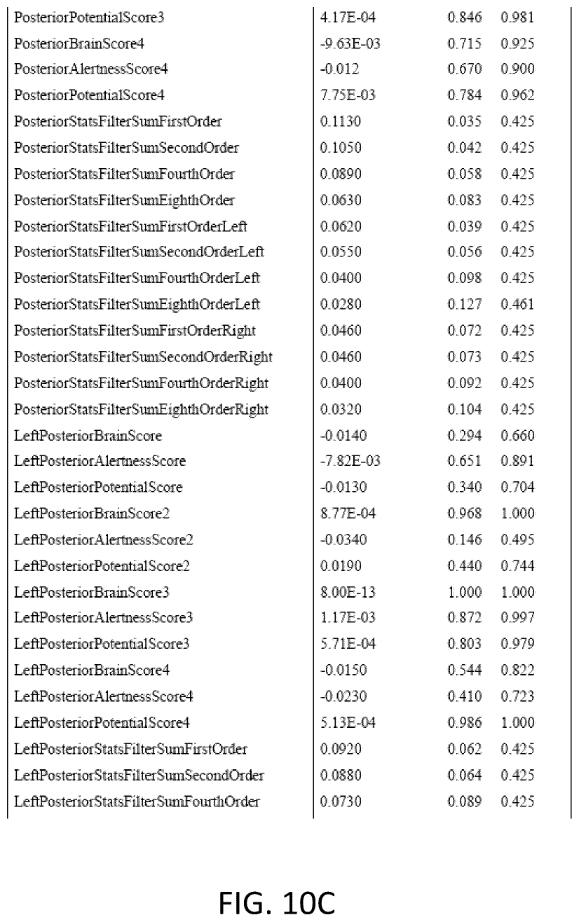

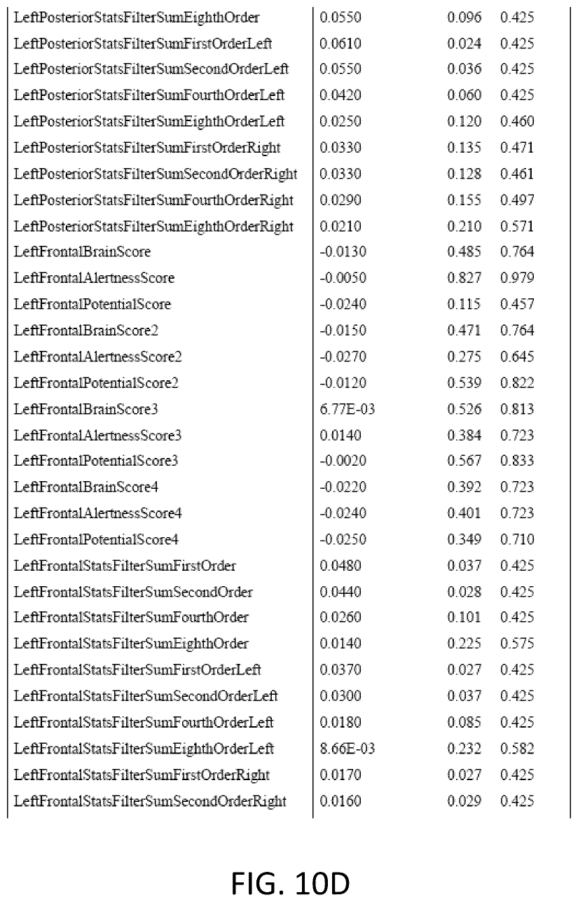

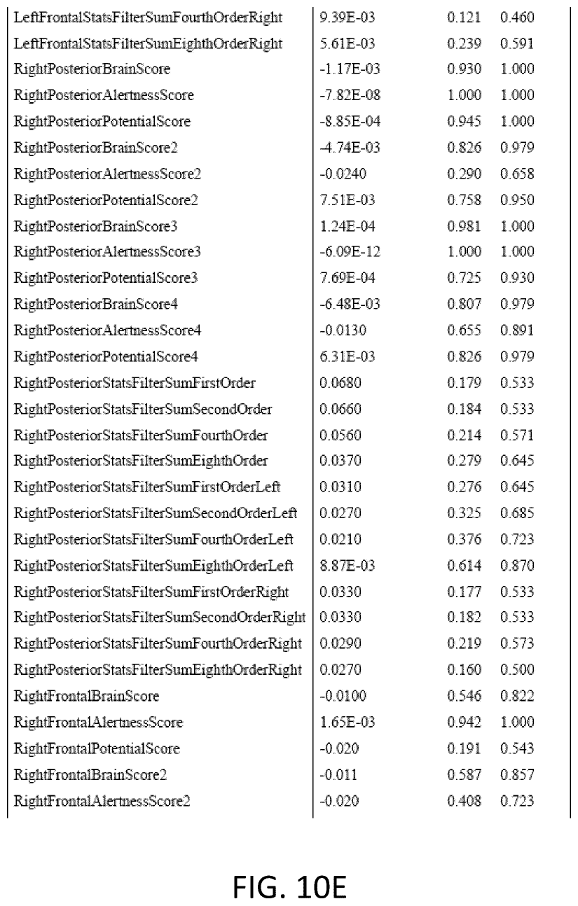

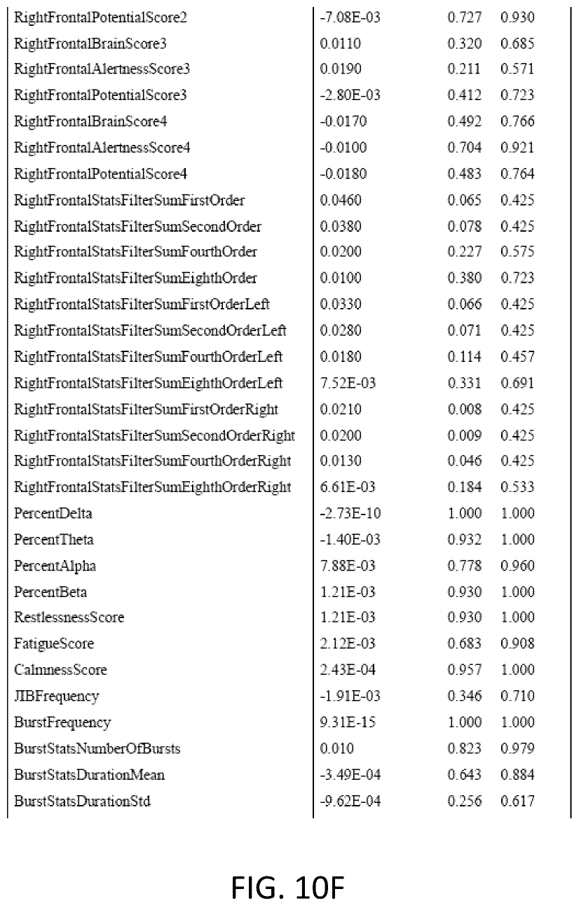

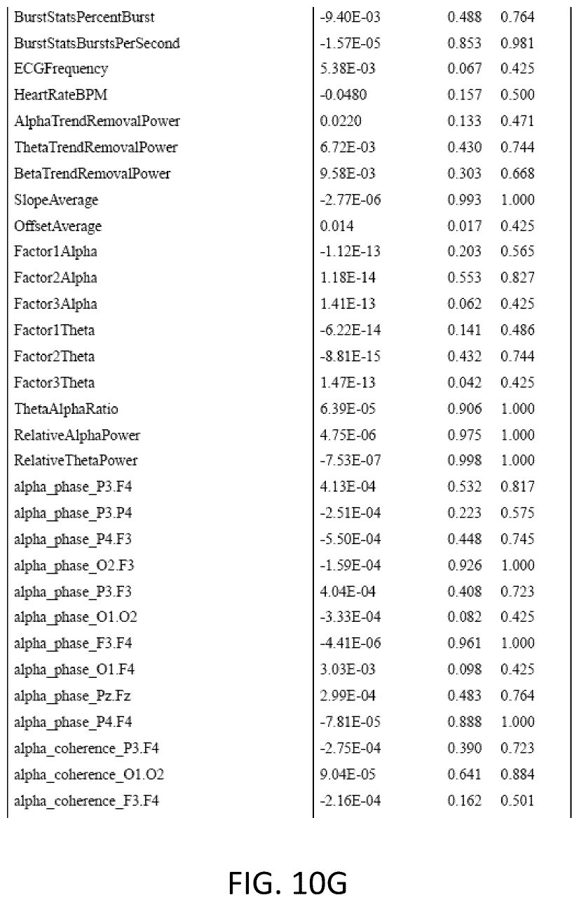

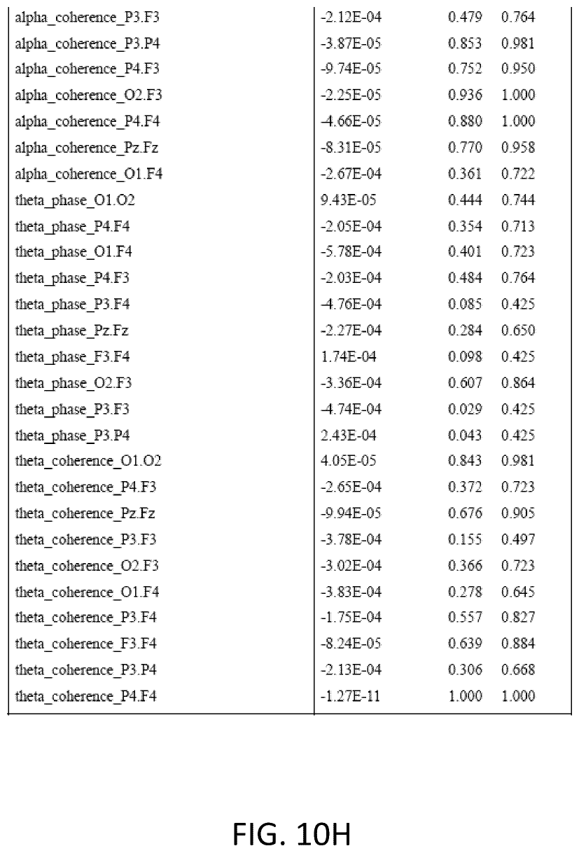

Results: Example results of first session one-sample tests are provided in FIGS. 9A-9H. All tests were one-sample Wilcoxon tests. A positive mean change indicates an overall improvement in patients during the first session with respect to that variable, assuming larger variable values are better. Variables with adjusted p-values less than 0.05 were FrontalBrainScore3 (mean change=0.373, p_{adj}=0.04), FrontalPotentialScore3 (mean change=0.213, p_{adj}=0.005), LeftFrontalPotentialScore3 (mean change=0.200, p_{adj}=0.044), RightFrontalPotentialScore3 (mean change=0.223, p_{adj}=0.0002). Also worth noting are GlobalPotentialScore3 (mean change=0.180, p_{adj}=0.051) and SlopeAverage (mean change=−0.010, p_{adj}=0.054). None of the coefficient estimates for treatment had adjusted p-values below 0.05 (FIGS. 10A-10H) in rank-based multiple regression.

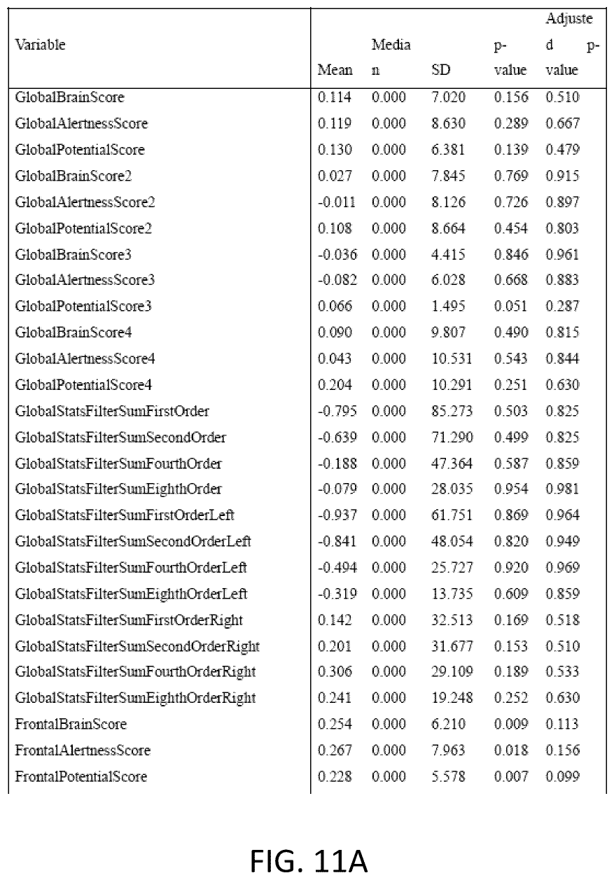

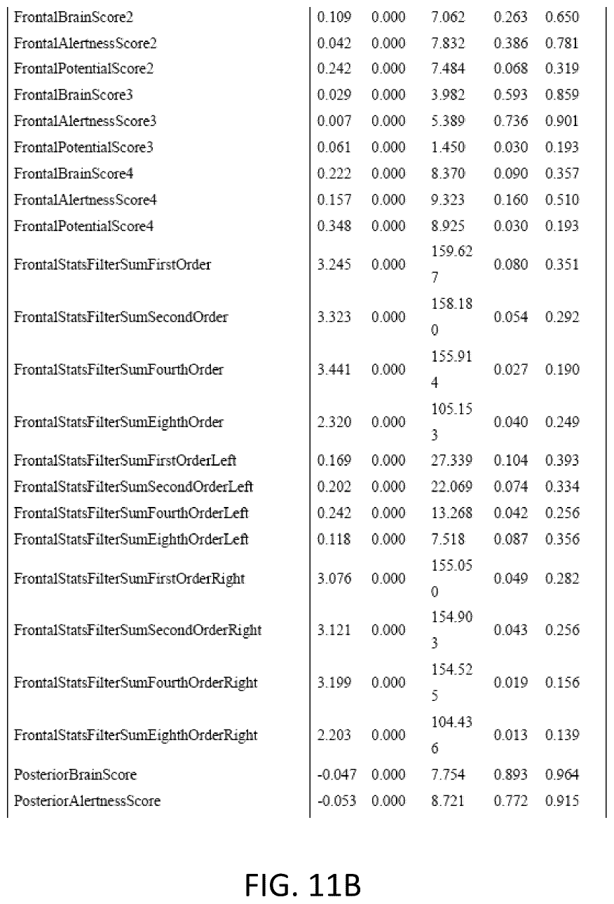

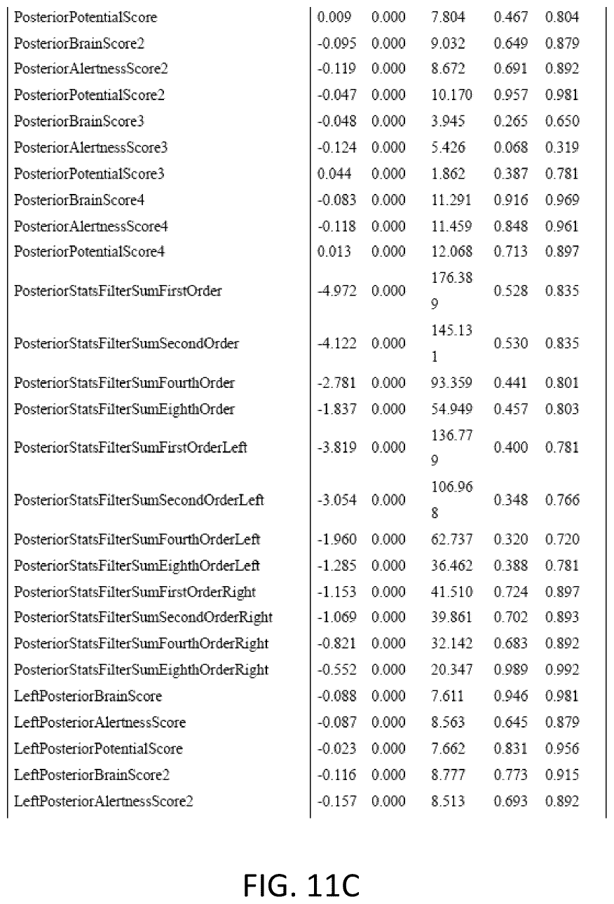

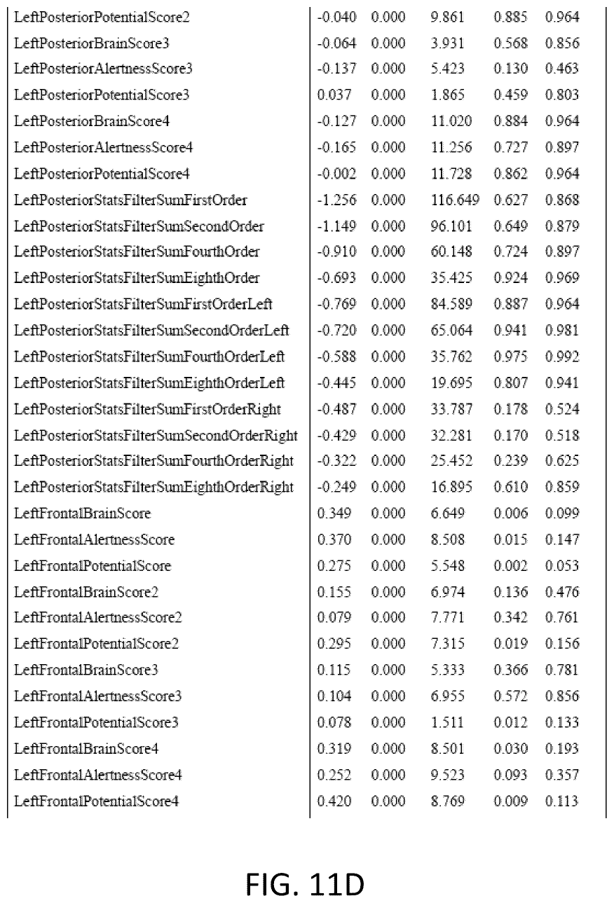

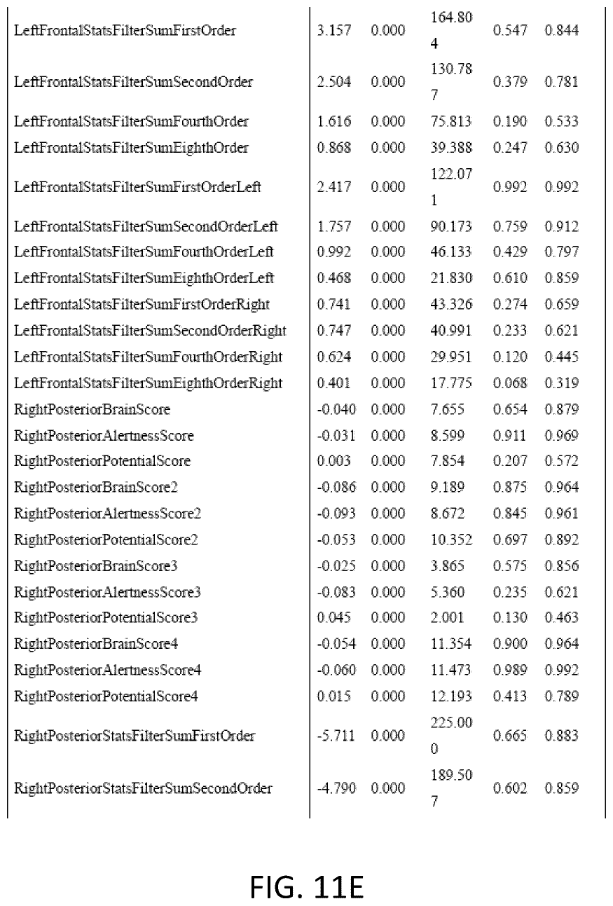

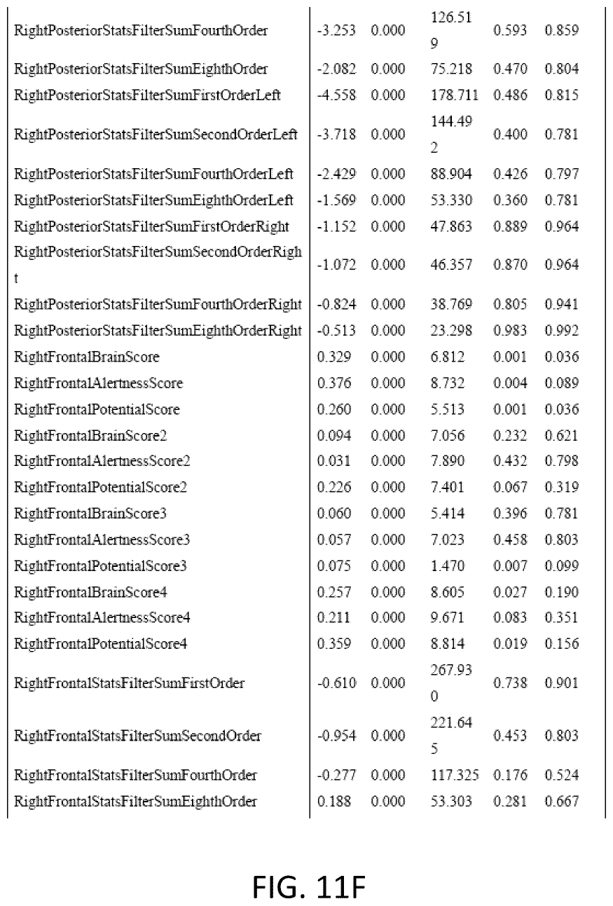

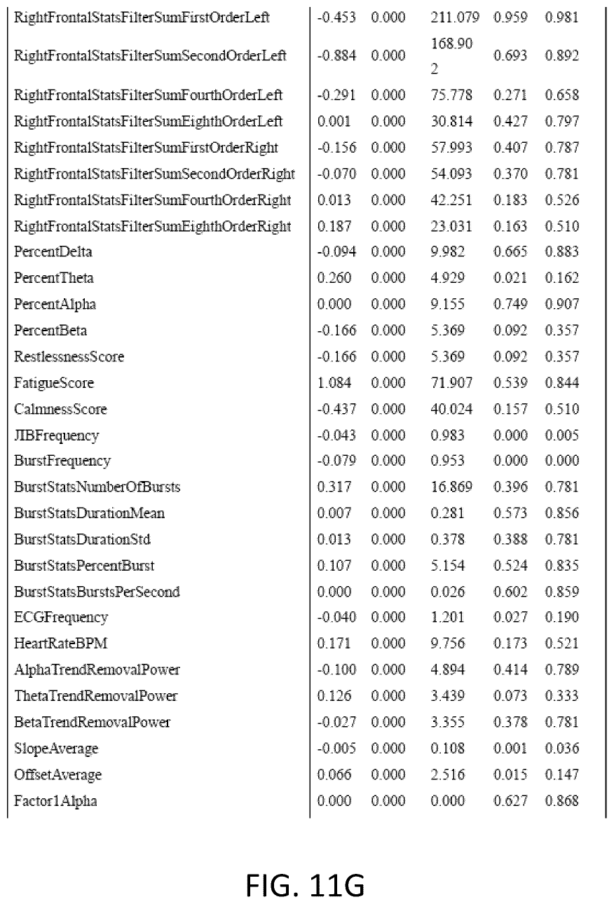

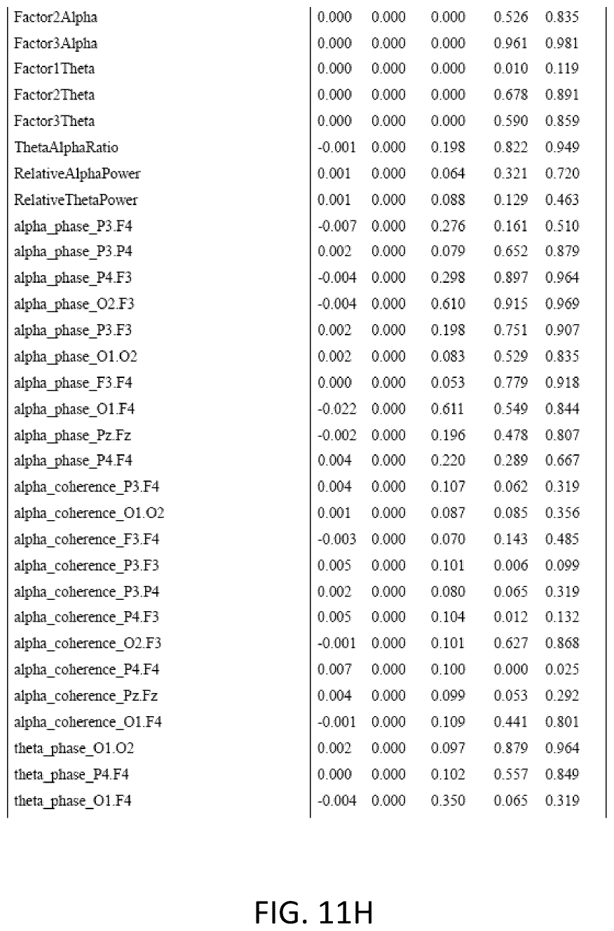

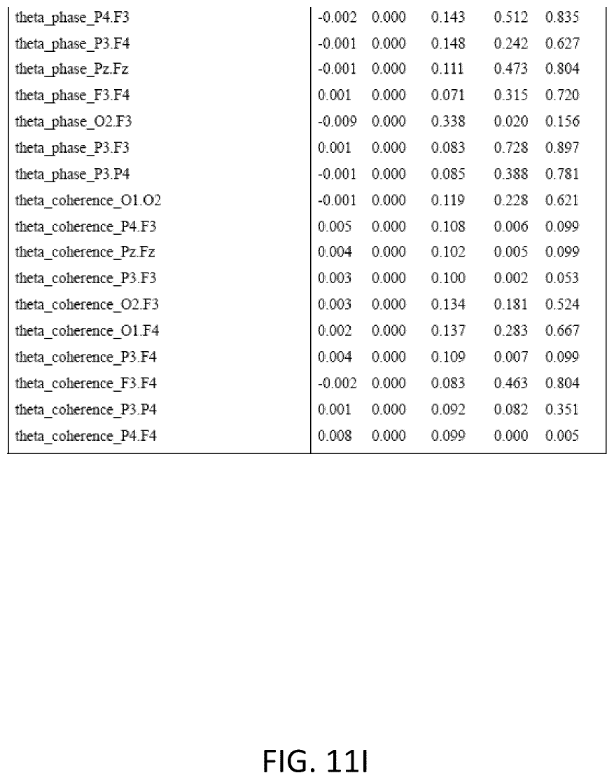

Example results of full treatment history one-sample test are provided in FIGS. 11A-11I. All tests were one-sample Wilcoxon tests. A positive mean change indicates an overall improvement in patients during the full course of treatment with respect to that variable, assuming larger variable values are better. Variables with adjusted p-values less than 0.05 were RightFrontalBrainScore (mean change=0.329, p_{adj}=0.036), RightFrontalPotentialScore (mean change=0.260, p_{adj}=0.036), JIBFrequency (mean change=−0.043, p_{adj}=0.005), BurstFrequency (mean change=−0.079, p_{adj}=0.00012), SlopeAverage (mean change=−0.005, p_{adj}=0.036), alpha_coherence_P4-F4 (mean change=0.007, p_{adj}=0.025), theta_coherence_P4-F4 (mean change=0.008, p_{adj}=0.005). Also worth noting are theta_coherence_P3-F3 (mean change=0.003, p_{adj}=0.053) and LeftFrontalPotentialScore (mean change=0.275, p_{adj}=0.053).

Feature Extraction

Introduction: This analysis determines a subset of variables that are relevant and useful for feature selection, and then performs an EFA and CFA using the subset of relevant and useful variables. The CFA scores can then be used as a multivariate score for downstream analyses.

Data Organization: All data cleaning and manipulation were performed using the statistical programming language R (R Core Team 2020). The data was filtered such that FailedArtifactor was FALSE, age was higher than 18 years, GlobalBrainScore was greater than or equal to 50, and LowVoltage was FALSE. Any observations without a recording date were removed. A new variable providing the time in days from the start of treatment was defined. Any EEGs that were repeated on the same day with no change in cortical counts (no treatment occurred) were removed. A variable was set up to define whether a patient took a break of 6 weeks during the course of treatment. Any length of time not separated by a 6-week break was called a treatment session, and a new variable was defined to indicate treatment session (the first session was labeled session 0, a baseline). A second time variable was set up that provided the days from the start of the session. Finally, a variable was set up that defined the cumulative cortical count. The data was filtered such that only patients with a maximum of 45 treatments over a maximum of 100 days during the first (baseline) treatment session were selected. For all variables of interest, baseline and final scores were stored as variables. The difference between the final and baseline score for all variables was also stored.

The data was separated into layers. For baseline, final, and difference scores, layers were defined using brain score, alertness score, potential score, brain score 2, alertness score 2, potential score 2, brain score 3, alertness score 3, potential score 3, first order, second order, fourth order, eighth order, coherence, phase, power, gender, diagnosis not available, neurological diagnosis, psychological diagnosis, substance abuse, miscellaneous, screening and optimization, and final a layer for all other variables. Musculoskeletal and circulatory diagnoses were not included because most or all patients fell into the FALSE category. All layers excluding those with factor variables were standardized. This resulted in 58 layers organized into a list.

A preliminary super-organizing map was fit on this list using a 13 by 13 hexagonal toroidal grid in the R package kohonen. Map quality was assessed using count, quality, and neighbor distance plots. The codebook vectors were bound into a single data frame and an optimal number of clusters was identified using the silhouette and gap statistic methods and clustering using medoids. All clusters with less than 5 items per cluster were removed as outliers.

The data in the list was then split into a training and test set such that 50% of the observations were in each set. The training set was used to train a second super-organizing map and perform exploratory analysis. Factor analysis was performed on the set of initial EEGs, leaving final scores out such that the CFA scores can be used to track patient progress.

Cluster Identification with Self-Organizing Maps: A super-organizing map was fit on the training data using a 9 by 9 hexagonal toroidal grid in the R package. Map quality was assessed using count, quality, and neighbor distance plots. The codebook vectors were bound into a single data frame and an optimal number of clusters was identified using the silhouette and gap statistic methods and clustering using medoids. To obtain a set of variables relevant for defining clusters, (1) the codebook vectors for each cluster were examined, (2) a random forest was fit to classify the data to the SOM-generated clusters and the importance plot was examined, and (3) a conditional inference tree was fit using the R package partykit. Based on these measures of importance, a subset of variables was selected for use in EFA. Specifically, all variables used in the conditional inference tree were collected, along with the top 5 important variables used to construct the random forest as identified by mean decrease in accuracy and mean decrease in Gini (10 variables total), and the top 5 variables with high median codebook vectors in each cluster were added to this set. Unique variables in the set were retained and any factor variables were removed. This was the final set of important variables used for EFA.

Identification of Supplementary Important Variables: The above methodology identified variables that are important in clustering patients into groups, or specifically in identifying differences between patients. To supplement these, variables of global importance were also identified. The variables included for this purpose were Factor1Alpha, Factor2Alpha, Factor3Alpha, Factor1Theta, Factor2Theta, Factor3Theta, ThetaAlphaRatio, RelativeAlphaPower, and RelativeThetaPower.

Exploratory Factor Analysis: The first EEG for each patient was extracted and the data set was randomly divided 50/50% into a training set for EFA and an independent test set for CFA. The important variables identified were selected. The training set was assessed for normality and transformations to achieve normality were attempted via Box-Cox and standard transformations (e.g., log, logit, cube root, etc.). The correlation matrix was inspected visually and using the Kaiser, Meyer, and Olkin (KMO) Measure of Sampling Adequacy (MSA) in the R package psych. Highly correlated variables were removed until a KMO>0.8 was achieved and variable correlations were moderate. The number of factors were estimated using parallel analysis in the R package psych. Exploratory factor analysis was then performed using the number of factors determined by parallel analysis and varimax rotation using the fa function psych. Factor scores were output as Excel files for interpretation using the R package readxl. The variable with the highest loading on a factor was assigned to that factor. A CFA model was written to reflect the results of the EFA.

Confirmatory Factor Analysis: A standard confirmatory factor analysis model was fit to the data using the R package lavaan with the latent variable variance constrained to 1 for the model specified by the EFA. Variables were scaled as needed to keep variances constant. The CFA was assessed for adequacy using the root mean square error of approximation (RMSEA), Comparative Fit Index (CFI), and Tucker-Lewis Index (TLI). Factor scores were predicted on the full data set for each patient that had non-NA values for the variables in the CFA model. A multivariate score was generated by taking the average of the factor scores. The F1-F3 and multivariate score were visualized using line plots and tile plots. To provide an alternative to taking the mean of F1-F3, the multivariate score was generated using two linear combinations of F1-F3 and included in the plots.

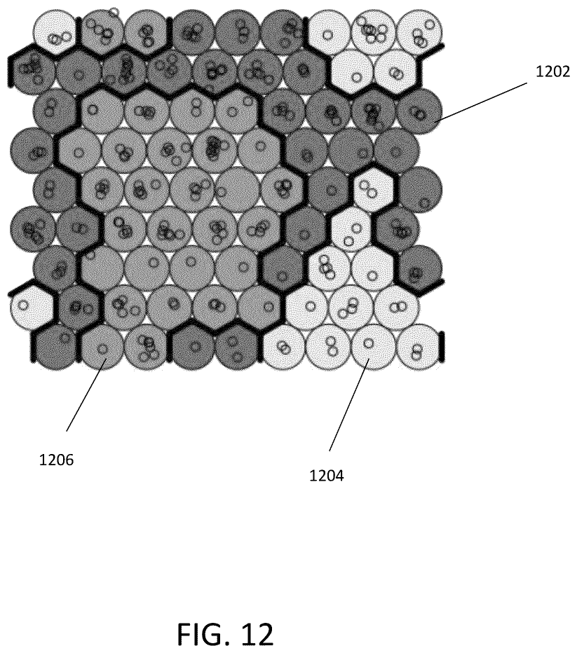

Results: The preliminary super-organizing map to identify outliers was fit using 800000 iterations on the full data set and did not demonstrate any diagnostic issues. 6 clusters were used to classify the nodes. 7 outlier nodes containing 13 outlier observations were removed. The super-organizing map fit on training data is provided in FIG. 12 with 3 clusters 1202, 1204, and 1206 used to classify the nodes. Important variables as selected using the codebook vectors of the super-organizing map included: FrontalBrainScore2_Baseline, RightFrontalBrainScore2_Baseline, FrontalAlertnessScore2_Baseline, LeftFrontalBrainScore2_Baseline, RightFrontalAlertnessScore2_Baseline, Diagnosis Neurological=FALSE, Diagnosis Other Miscellaneous=FALSE, Diagnosis Substance Use=FALSE, Diagnosis Psychological=FALSE, Diagnosis Not Available=FALSE, Diagnosis Screening And Optimization=FALSE, and Gender=0.

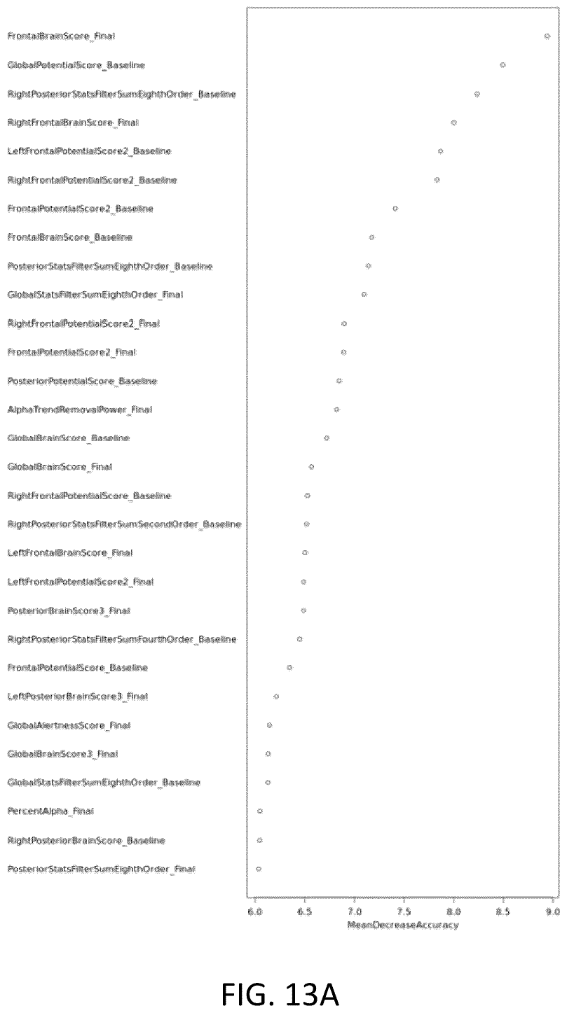

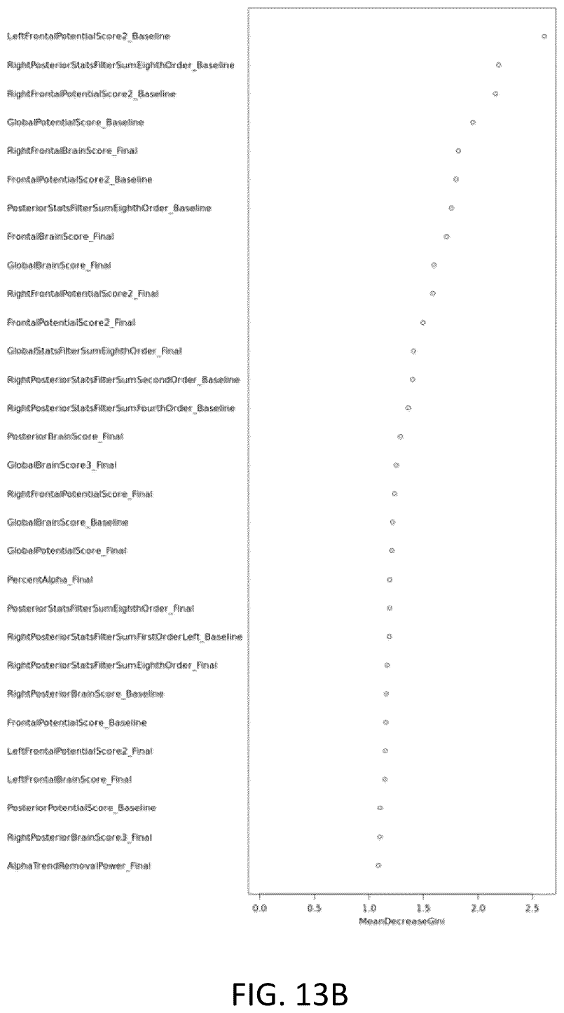

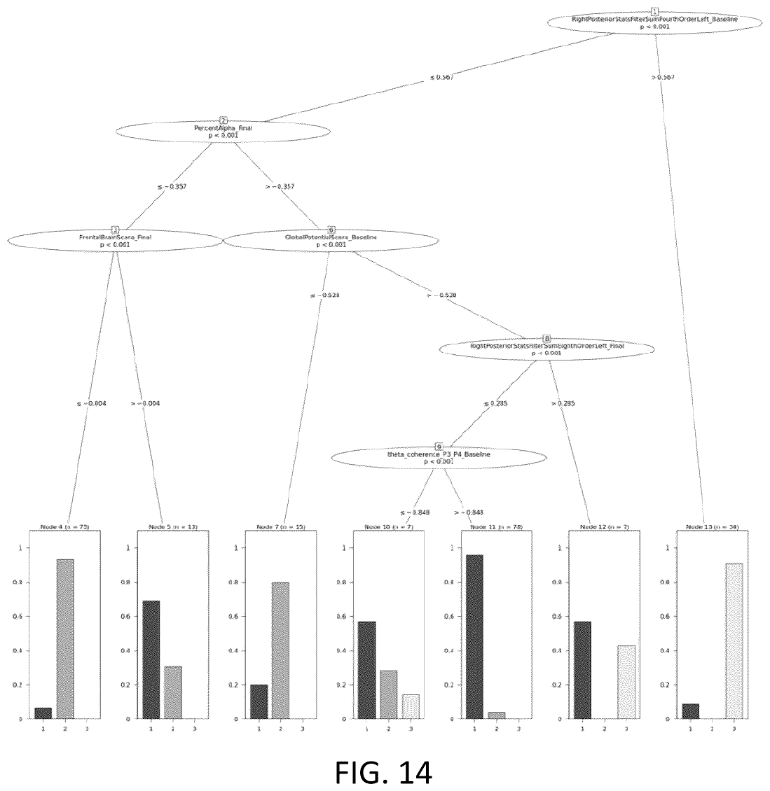

The variable importance plot for the random forest is shown in FIGS. 13A, 13B. The set of important variables given the random forest fit included: FrontalBrainScore_Final, GlobalPotentialScore_Baseline, RightPosteriorStatsFilterSumEighthOrder_Baseline, RightFrontalBrainScore_Final, LeftFrontalPotentialScore2_Baseline, and RightFrontalPotentialScore2_Baseline. The fitted conditional inference tree is shown in FIG. 14. The variables utilized to fit the conditional inference tree were RightPosteriorStatsFilterSumFourthOrderLeft_Baseline, PercentAlpha_Final, FrontalBrainScore_Final, GlobalPotentialScore_Baseline, RightPosteriorStatsFilterSumEighthOrderLeft_Final, and theta_coherence_P3_P4_Baseline.

These variables combined with the supplementary variables were used for exploratory factor analysis. The baseline/final distinction was removed when including important variables. EFA was fit on complete cases (n=672). Specifically, the final list of important variables was: FrontalAlertnessScore2, LeftFrontalBrainScore2, RightFrontalAlertnessScore2, FrontalBrainScore, RightPosteriorStatsFilterSumEighthOrder, RightFrontalBrainScore, LeftFrontalPotentialScore2, RightFrontalPotentialScore2, RightPosteriorStatsFilterSumFourthOrderLeft, PercentAlpha, GlobalPotentialScore, RightPosteriorStatsFilterSumEighthOrderLeft, theta_coherence_P3-P4, Factor1Alpha, Factor2Alpha, Factor3Alpha, Factor1Theta, Factor2Theta, Factor3Theta, ThetaAlphaRatio, RelativeAlphaPower, and RelativeThetaPower.

Neither Box-Cox nor standard transformations resulted in data that adhered to assumptions of normality. Generally, the transformed distributions were not well-behaved at the tails, causing tests of normality to fail. When observations greater than 1 standard deviation from the mean were removed, univariate normality was a reasonable assumption, however, this removed a large portion of the data. In general, Box-Cox transformations performed better than standard transformations, but not well enough to maintain the observations in the tails.

The inspection of the correlation matrix and computation of the KMO MSA resulted in the removal of the following variables due to high correlations (>0.8, <−0.8): RightPosteriorStatsFilterSumEighthOrder, RightPosteriorStatsFilterSumFourthOrderLeft, RightPosteriorStatsFilterSumEighthOrderLeft, LeftFrontalBrainScore2, RightFrontalBrainScore2, RightFrontalBrainScore, RightFrontalAlertnessScore2, PercentAlpha, ThetaAlphaRatio, Factor3Alpha, Factor3Theta, FrontalBrainScore2, and Factor2Alpha.

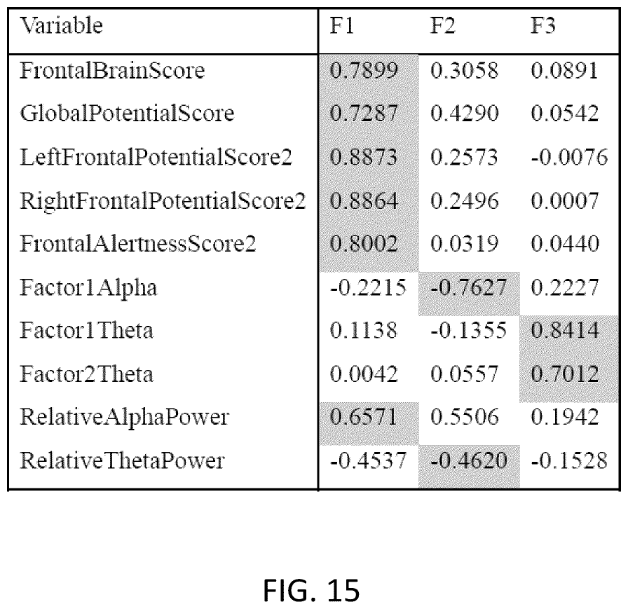

Additionally, theta_coherence_P3-P4 was removed because it loaded on a factor alone or the loading was very small. The final KMO MSA was 0.816. Since the data did not adhere to the assumption of normality, weighted least squares was used as the fitting method. Parallel analysis resulted in 3 factors. The factor loadings are shown in FIG. 15. Factors 1-3 explained 40.7%, 15.3%, and 13.2% of variance in the data, for a total of 69.2% of the variance. The root mean square of the residuals (RMSA) was 0.04 and the Tucker Lewis Index of factoring reliability was 0.427. Factor 1 (F1) was defined by the variables FrontalBrainScore, GlobalPotentialScore, LeftFrontalPotentialScore2, RightFrontalPotentialScore2, FrontalAlertnessScore2, and RelativeAlphaPower. Factor 2 (F2) was defined by the variables Factor1Alpha and RelativeThetaPower. Factor 3 (F3) was defined by the variables Factor1Theta and Factor 2Theta.

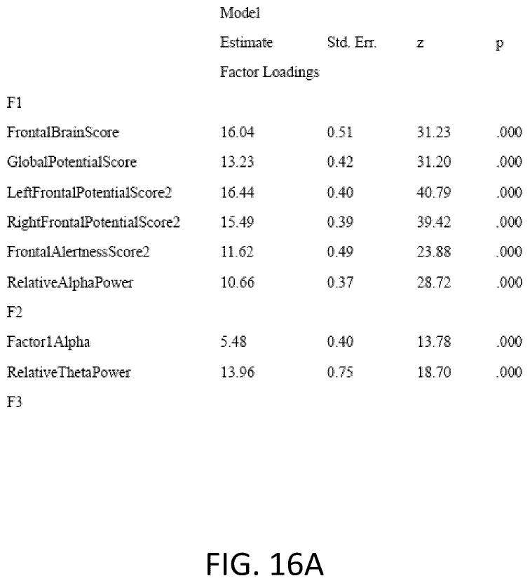

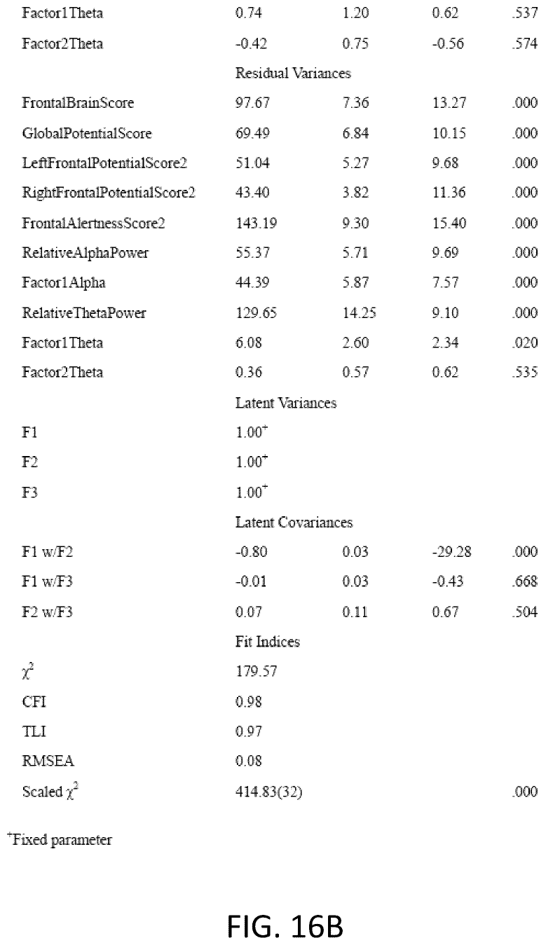

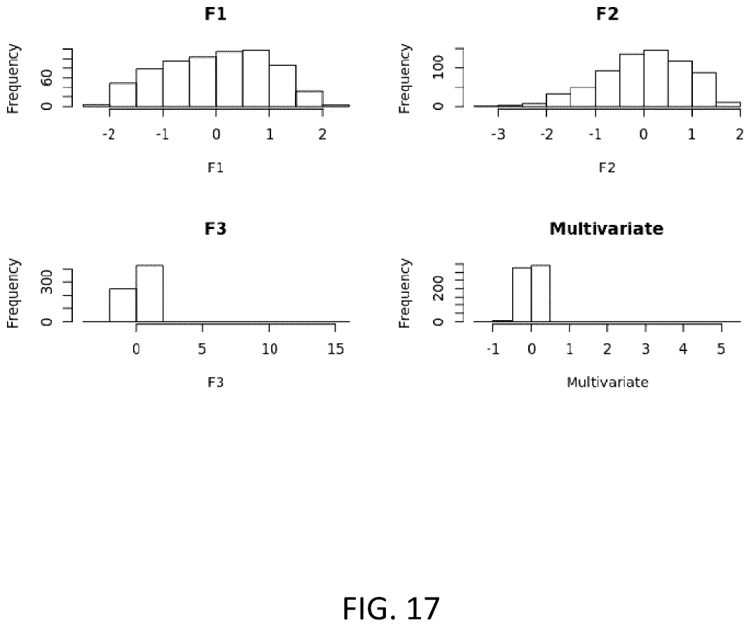

CFA was fit on complete cases of the test set (n=681). Due to issues estimating the variables, certain variables were scaled. Specifically, FrontalBrainScore, GlobalPotentialScore, LeftFrontalPotentialScore2, RightFrontalPotentialScore2, and FrontalAlertnessScore2 were divided by 100 and Factor1Alpha, Factor1Theta, and Factor2Theta were multiplied by 109. The model was fitted using the WLSM (robust weighted least squares) estimator and the summary is provided in FIGS. 16A, 16B. CFI=0.98, TLI=0.97, and RMSEA=0.08 suggest good model fit. Distributions of predicted scores as well as the multivariate score (average of factor scores) for the test set are provided in FIG. 17. The distribution of F3 appears to be impacted by extreme values and influences the distribution of the multivariate score.

F1-F3 and multivariate scores were predicted for all EEGs/patients who had non-NA values of the variables in the CFA model. These are available for review and examination to identify any potential issues or possibilities for improvement in this EFA/CFA analysis.

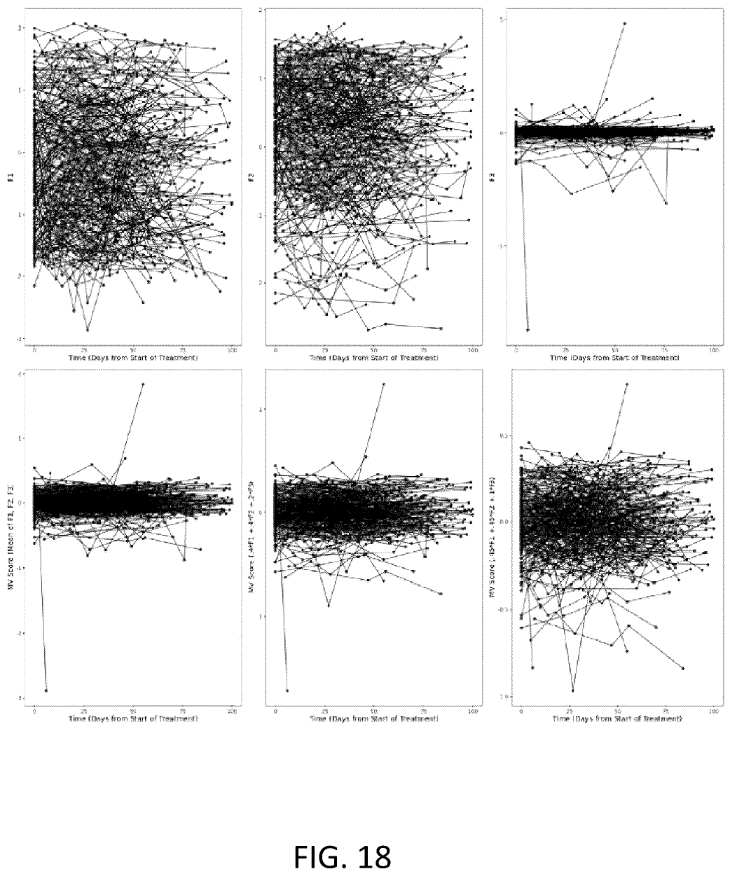

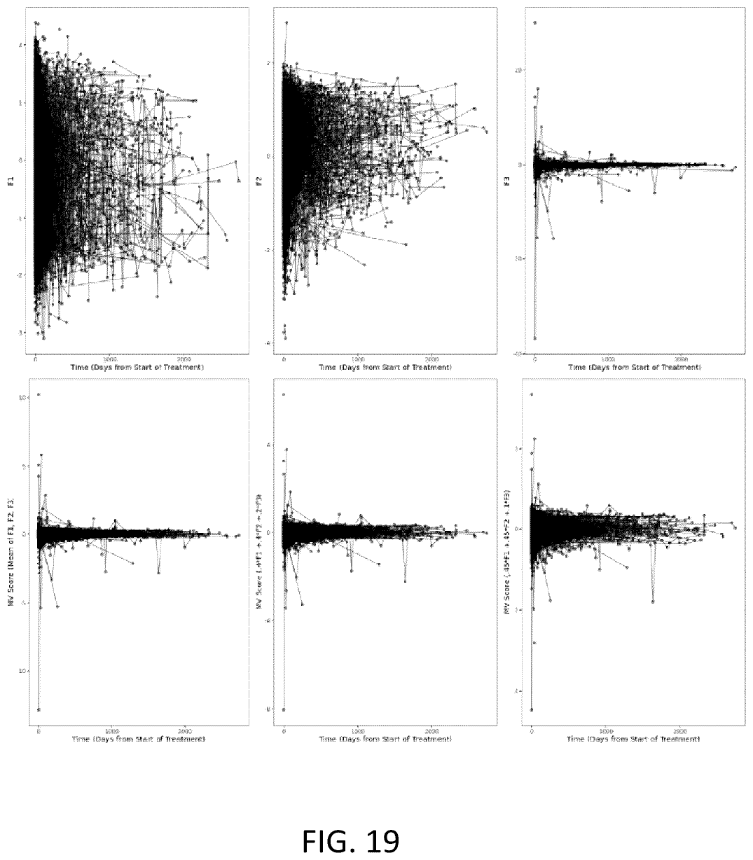

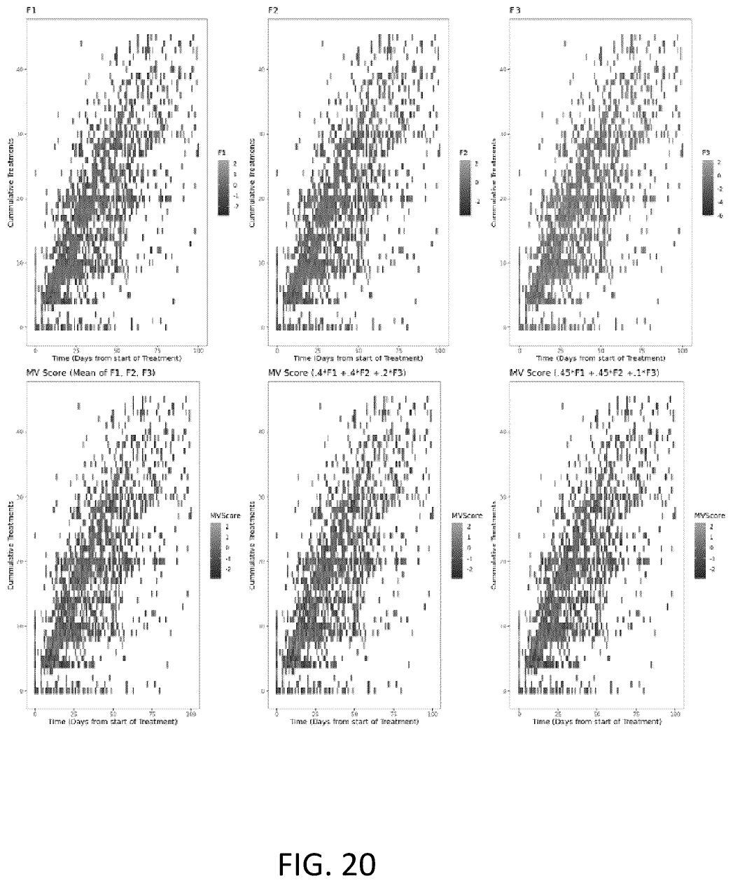

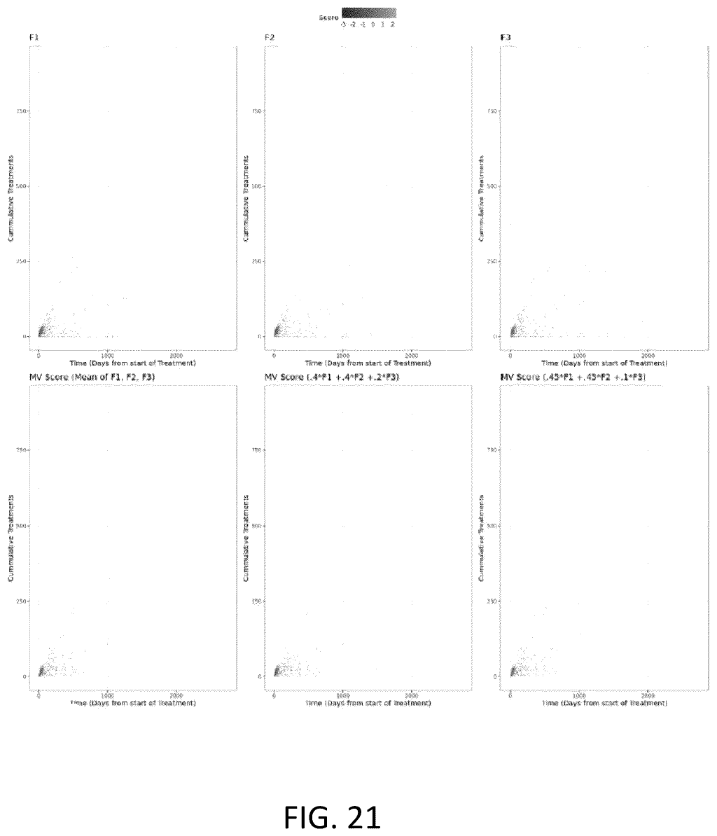

Line plots of F1-F3 and the multivariate expressed as an average and two linear combinations are provided in FIGS. 18 and 19 for the first session and full data set, respectively. Corresponding tile plots are provided in FIGS. 20 and 21 for the first session and full data set, respectively.

Updated Factor Analysis

Introduction: This analysis performs EFA and CFA using local variables in place of previous variables and examines changes.

Statistical Methodology: All analyses were completed using the statistical programming language R (R Core Team 2020). EFA proceeded with the variables used for previous EFA. The first EEG for each patient was extracted and the data set was randomly divided 50/50% into a training set for EFA and an independent test set for CFA. The correlation matrix was inspected visually and using the Kaiser, Meyer, and Olkin (KMO) Measure of Sampling Adequacy (MSA) in the R package psych. The number of factors were estimated using parallel analysis in the R package psych. EFA was then performed using the number of factors determined by parallel analysis and varimax rotation using the fa function psych. Factor scores were output as Excel files for interpretation using the R package readxl. The variable with the highest loading on a factor was assigned to that factor. A CFA model was written to reflect the results of the EFA.

A standard CFA model was fit to the data using the R package lavaan with the latent variable variance constrained to 1 for the model specified by the EFA. Variables were scaled as needed to keep variances constant. The CFA was assessed for adequacy using the root mean square error of approximation (RMSEA), Comparative Fit Index (CFI), and Tucker-Lewis Index (TLI).

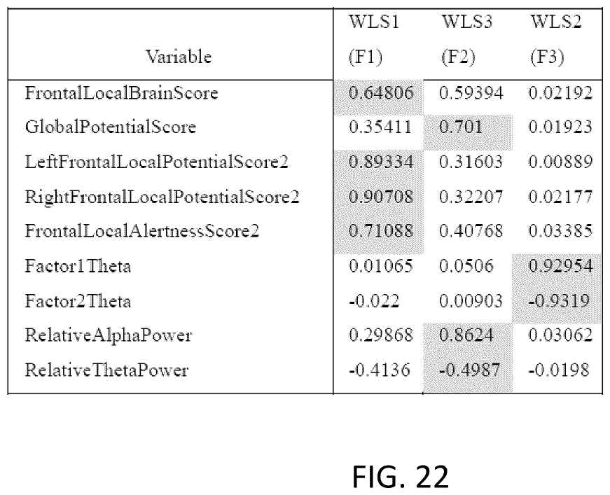

Results: The EFA and CFA were fit without Factor1Alpha because inclusion of this variable generated a negative estimated variance in the CFA. The KMO Measure of Sampling Adequacy was 0.802 for the EFA data set (n=824). Since the data did not adhere to the assumption of normality, weighted least squares was used as the fitting method. Parallel analysis resulted in 3 factors. The factor loadings are shown in FIG. 22. Factors 1-3 explained 33%, 25%, and 19% of variance in the data, for a total of 76% of the variance. The root mean square of the residuals (RMSA) was 0.02 and the Tucker Lewis Index of factoring reliability was 0.867.

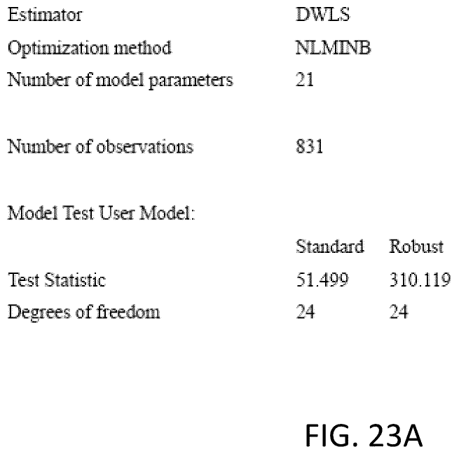

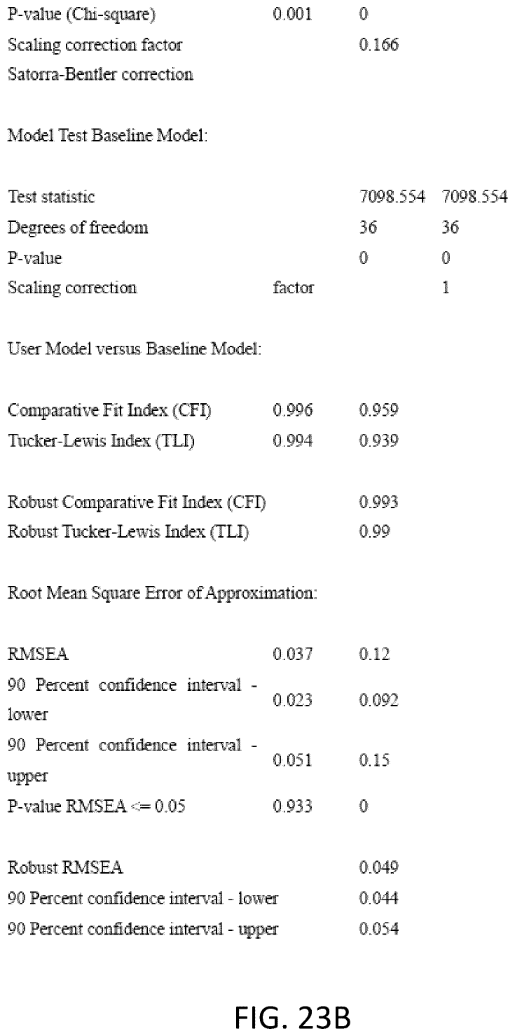

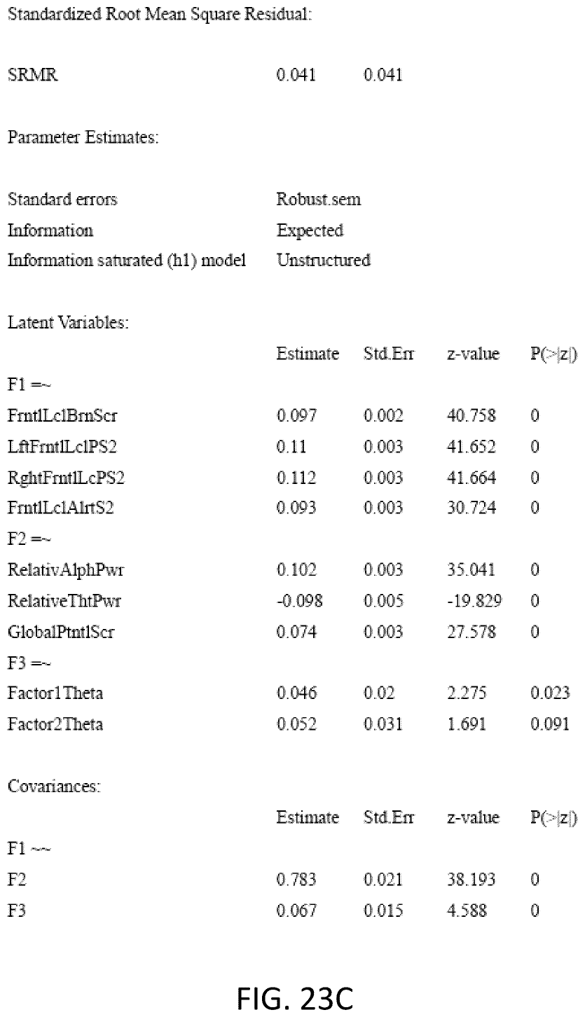

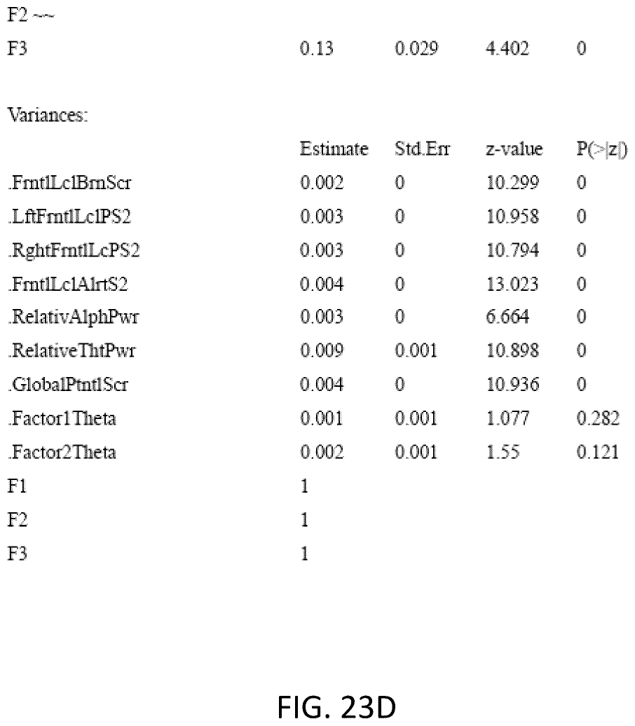

Factor 1 (F1) was defined by the variables FrontalLocalBrainScore, LeftFrontalLocalPotentialScore2, RightFrontalLocalPotentialScore2, and FrontalLocalAlertnessScore2. Factor 2 (F2) was defined by the variables GlobalPotentialScore, RelativeAlphaPower, and RelativeThetaPower. Factor 3 (F3) was defined by the variables Factor1Theta and Factor 2Theta. The CFA model was fitted using the WLSM (robust weighted least squares) estimator and the summary is provided in FIGS. 23A-23D. CFI=0.996, TLI=0.994, and RMSEA=0.037 suggest a superior fit to the previous model.

In some examples, based on the EFA/CFA, a transfer function is created with multiple factors (e.g., two, three, four, five, etc.) that, when combined, explain more than about 80 percent of the variants of the pre/post change. This transfer function represents a predetermined transfer function that is used to process EEG data collected from future patients/subjects. In some examples, the predetermined transfer function is updated (e.g., periodically) as new EEG data becomes available. In some examples, the output of the transfer function, also called the “solution” or “score” herein, contains information about FFT synchrony, relative amount of alpha versus other bands, first statistics, and other information. In some examples, each factor is based on multiple variables (e.g., two, three, four, five, etc.). In some examples, the contributions of the variables to each factor are determinate based on EFA/CFA.

For example, a four-factor solution may include 18 variables (more or less) that are clustered on each one of those variables. In some examples, the variance is accounted for in each factor. For example, about 30% for one factor, about 20% for two factors, about 10% for three factors, etc. In some examples, these factors are assigned relative weights (e.g., expressed as percentages) for calculating the Brain Synchrony Index (e.g., also called the “score” herein and/or Brain Synchrony Score (BSS)). In some examples, the BSS is normalized on about 50% to about 100% range.

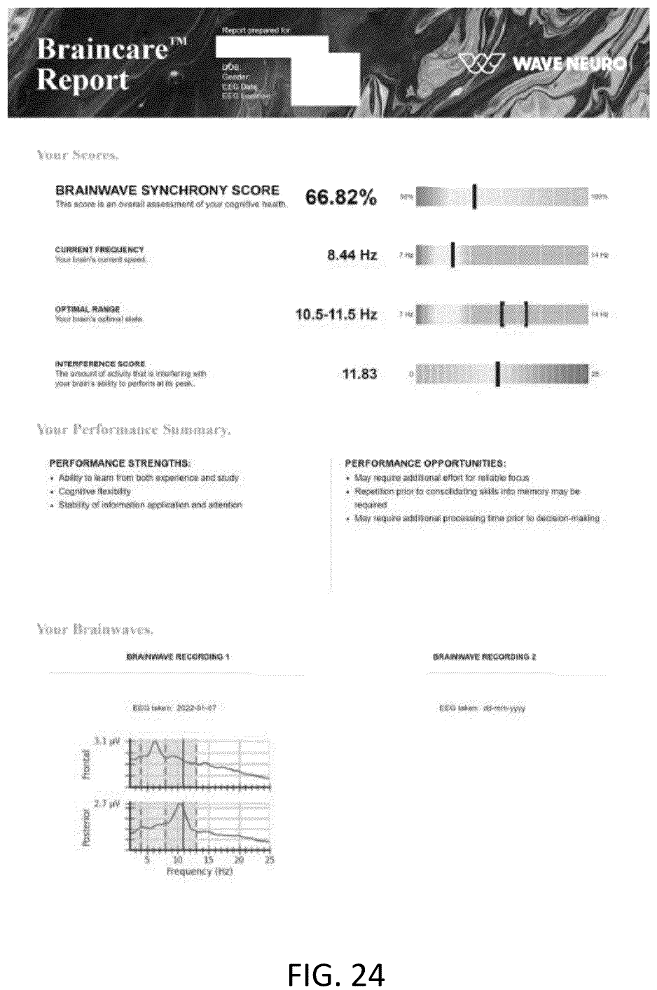

FIG. 24 is an example of a Braincare™ Report according to aspects described herein. In some examples, the BSS is depicted as a percentage (e.g., 66.82% in the example depicted in FIG. 24). In some examples, the BSS is depicted in a linear graph with shaded or color spectrum enhancement (e.g., as shown in FIG. 24).

In some examples, those who have a more synchronous alpha FFT peak with less theta and more alpha have a higher BSS than those who have more theta, less alpha, and a less sharp peak. Thus, the BSS may be an aggregated score as shown in FIG. 24.

In some examples, a BSS of at least about 75% is considered to be “good.” Likewise, a BSS that is less than about 75% may be less synchronous. Another number that may be reported is “current frequency.” In some examples, a current frequency above about 8 Hz is considered “good.” The current frequency is depicted in Hertz (e.g., 8.44 Hz in the example depicted in FIG. 24). In some examples, the current frequency is depicted in a linear graph with shaded or color spectrum enhancement (e.g., as shown in FIG. 24). In some examples, the “Optimal Range” is also be depicted in Hertz (e.g., about 10.5 Hz-about 11.5 Hz in the example depicted in FIG. 24). In some examples, the Optimal Range is depicted in a linear graph with shaded or color spectrum enhancement (e.g., as shown in FIG. 24).

Another reported number, “interference score,” may be an indication of relative theta density. In some examples, relative theta density is determined as the quotient of the dividend, Theta, and the divisor (e.g., two). In some examples, a score produces a value between about 0 and about 50. It may be equal to about half the relative theta density. For example, theta density may be theta/2. Relative theta density may be equal to a power in a theta band divided by an overall EEG power. If all energy is in the theta band, the relative theta density may be about 100%, and the interference score may be about 50. In some examples, the system monitors for a low score (e.g., 5-8 may be relatively common). If the score is above about 10, then patients may often experience memory problems.

In some examples, Theta is an indicator of slow wave activities. Theta may be in a band between about 5 Hz to about 7 Hz. In some examples, excess Theta while the subject is awake may be an indicator of brain health problems.

FIG. 24 also provides frequency domain versions of brainwave recordings at one or more dates.

As described above, the BSS is determined from a transfer function that is based on the EFA/CFA described herein. The BSS is advantageous, at least in part, because the factor analysis is performed on a large database of pre/post stimulated EEGs with linked clinical outcomes. As disclosed herein, when the BSS moves from about 66% to about 70%, there may be a significant result. In some examples, more than about 85% of variants are accounted for with the EFA/CFA described in the disclosure. Because of the large database, the statistical confidence in the results is high.

In some examples, the BSS is determined from specific factors (e.g., F1, F2, F3, etc.). The relative contribution (weight) of each factor is determined from the EFA/CFA. In some examples, each factor is determined from specific variables that are determined from analysis of an EEG (or multiple EEGs). For example, as shown in FIG. 22 and described above in the example “Updated Factor Analysis”, Factor 1 (F1) may be defined by the variables FrontalLocalBrainScore, LeftFrontalLocalPotentialScore2, RightFrontalLocalPotentialScore2, and FrontalLocalAlertnessScore2. Factor 2 (F2) may be defined by the variables GlobalPotentialScore, RelativeAlphaPower, and RelativeThetaPower. Factor 3 (F3) may be defined by the variables Factor1Theta and Factor2Theta. In some examples, the transfer function includes a 3 Factor Solution, a 4 Factor Solution, a 5 Factor Solution, or any number of factors produced by the EFA/CFA. Note that the factors such as F3 are distinguished from the code for electrode placement in the 10-20 system described above and shown in FIG. 1.

In standard EEG reports, relative power may be a normal measurement. In some examples, specific artifacts from an improperly placed lead are filtered out. In some examples, digital signal processors execute algorithms that automatically determine or identify what is truly brainwave activity and what is not brainwave activity. Ultimately, this makes the reported results relatively more clear and more usable by clinicians.

### Example Cases

It should be appreciated that the systems, methods, and techniques described herein may be used to treat, mitigate, resolve, or otherwise impact the symptoms and/or causes of various diseases and disorders. In some examples, stimulation treatment is applied in response to common disruptions associated with Autism spectrum disorders (ASD), Alzheimer's disease, traumatic brain injuries, attention deficit disorder (ADD), attention deficit-hyperactivity disorder (ADHD). In some examples, stimulation is applied in response to common endophenotypes (or disruptions) associated with anxiety, depression, and addiction. In some examples, stimulation is used in association with diagnoses that are assigned based on well-defined etiologies (e.g., Biological disorders: Alzheimer's Disease, Cerebral Palsy, Stroke, Seizure, Traumatic Brain Injury, Meningitis, and Chemo-Brain). In some examples, stimulation is used in association with diagnoses that are assigned based on symptom profiles (e.g., Depression, ADD, ADHD, ASD, Post-Traumatic Stress Disorder (PTSD), and developmental delay). In some examples, these diagnoses are characterized, tracked and explored through use of EEG with measures of entropy and metabolic inferences.

There are multiple ways to disrupt symmetry in the brain. Generally, a disruption of alpha band activity is accompanied by additional activities (or indicators). In some examples, such additional activity includes an abnormally high glucose metabolism. Possible disruptions that increase metabolism include excess diffuse fast rhythms (e.g., Alpha (8-13 Hz) and Beta (13-25 Hz), excess synchronous fast rhythms (e.g., Beta), and lack of symmetrical dominant activity (e.g., absence of Alpha rhythm or multiple Alpha rhythms). In some examples, higher glucose metabolism is noted in subjects with ADHD, Autism, anxiety class disorders, PTSD, and Schizophrenia. In some examples, such additional activity includes an abnormally low glucose metabolism. Possible disruptions that decrease metabolism include excess diffuse slow rhythms (e.g., Delta (1-4 Hz) and Theta (4-8 Hz), excess synchronous slow rhythms (e.g., Delta and Theta), and abnormally high density of synchronous Alpha activity. In some examples, lower glucose metabolism is noted in subjects with traumatic brain injuries, Alzheimer's disease, chemo brain, drug abuse, sleep disorders, ADD, depression, and post-stroke tissue. Examples of common profiles are provided in Table 1:

TABLE 1

| Diagnosis | Profile |
|---|---|
| ASD | Posterior alpha rhythm, attenuation frontally. |
| | Disruption in development of activities. |
| Anxiety-class disorders | Excessive Beta, disrupted compensation. |
| | Can be primary or downstream of disruption. |
| Depression | Abnormally high frontocentral alpha density, |
| | generally synchronous. Possibly due to |
| | excessive Theta, generally primary. |
| Developmental disorders | Sub-8 Hz peak frequency. |
| Alzheimer's, | Sub-8 Hz peak frequency. |
| Parkinson's, Dementia | |
| Brain injury | Theta or Delta focal to injury site, Alpha |
| | attenuated locally or globally. |

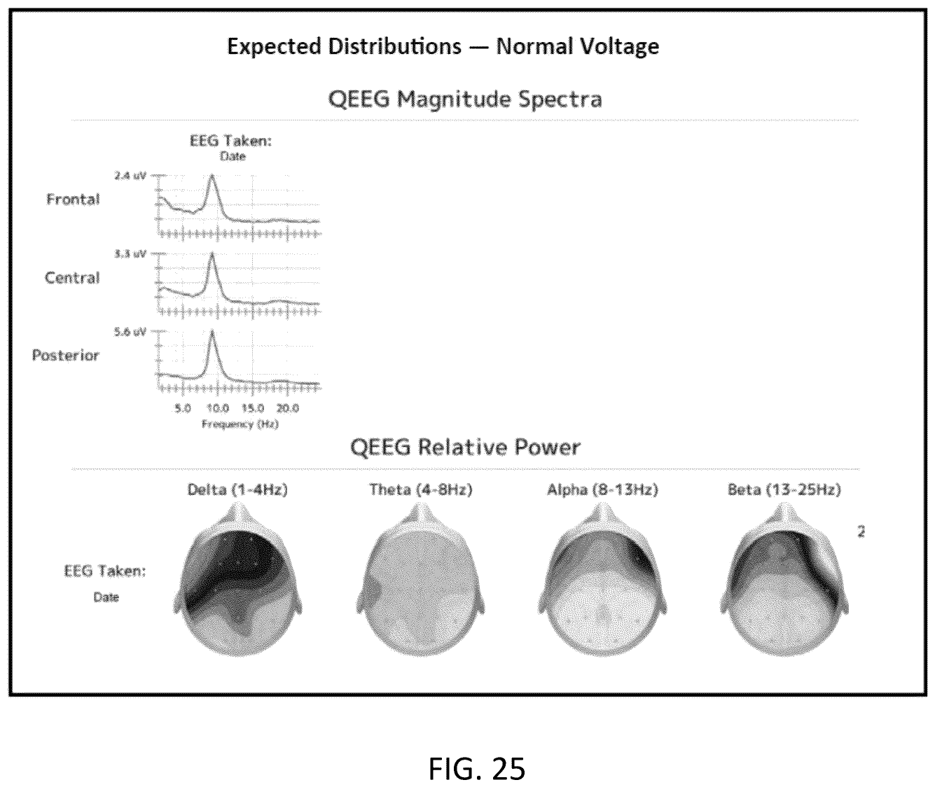

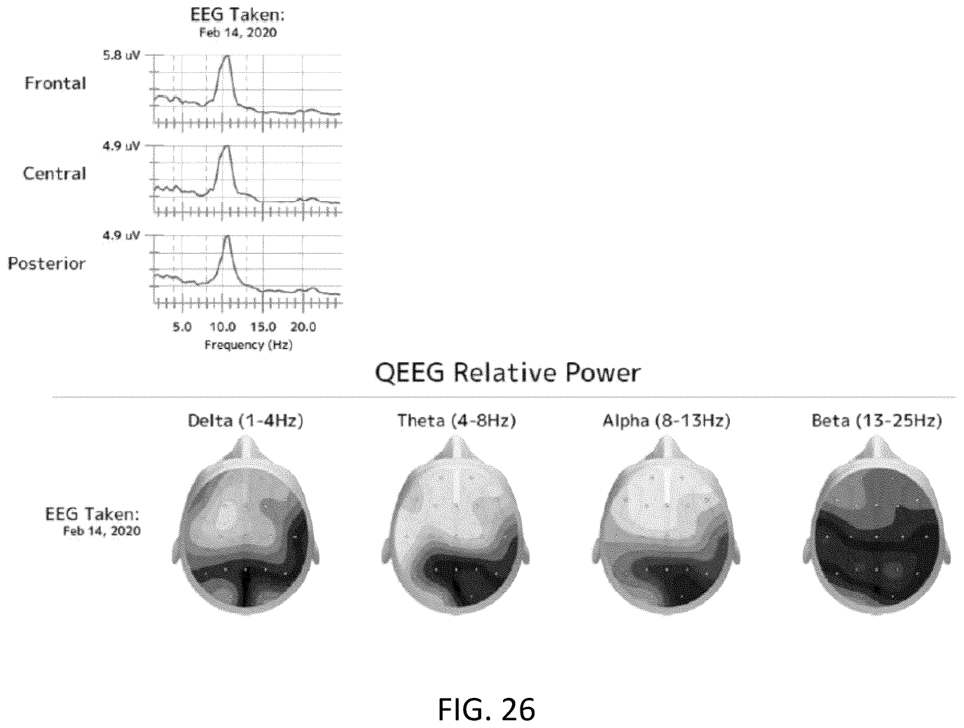

FIG. 25 is a quantitative EEG (QEEG) relative power measurement from a 45-year-old male subject with a Normal Voltage brain type without notable psychiatric, physiological, or neurocognitive issues. FIG. 26 is a similar relative power chart to FIG. 25, except FIG. 26 is for a different brain type, called Frontal Generator. A “Frontal Generator” as defined herein, may be a brain type where measured power in the alpha band as detected by the frontal electrodes (FP1, FP2) may be significantly above average. FIG. 26 is from a 35-year-old male with no presentation of psychiatric, physiological, or neurocognitive issues.

In QEEG Relative Power diagrams, e.g., the QEEG Relative Power diagrams in FIG. 25 and FIG. 26, each dot may represent an electrode on the head. In example embodiments, the data may be compared to a normative database of age-matched controls. For example, if the test subject is 30 years old, the test results may be compared against the normative values for a 30-year-old patient. The scale in the QEEG Relative Power diagrams may be in units of “Z”, which may be defined as one standard deviation. In example embodiments, a color or shade in the QEEG Relative Power diagrams may be indicative of a positive or negative quantity of standard deviations. In an example embodiment based on age: assuming the norm for alpha is 25% of the left frontal (F3) electrode; and assuming that one standard deviation is 5%; a test reading of 30% for the left frontal electrode may be interpreted as having alpha that is about one standard deviation higher than the norm (+1Z).

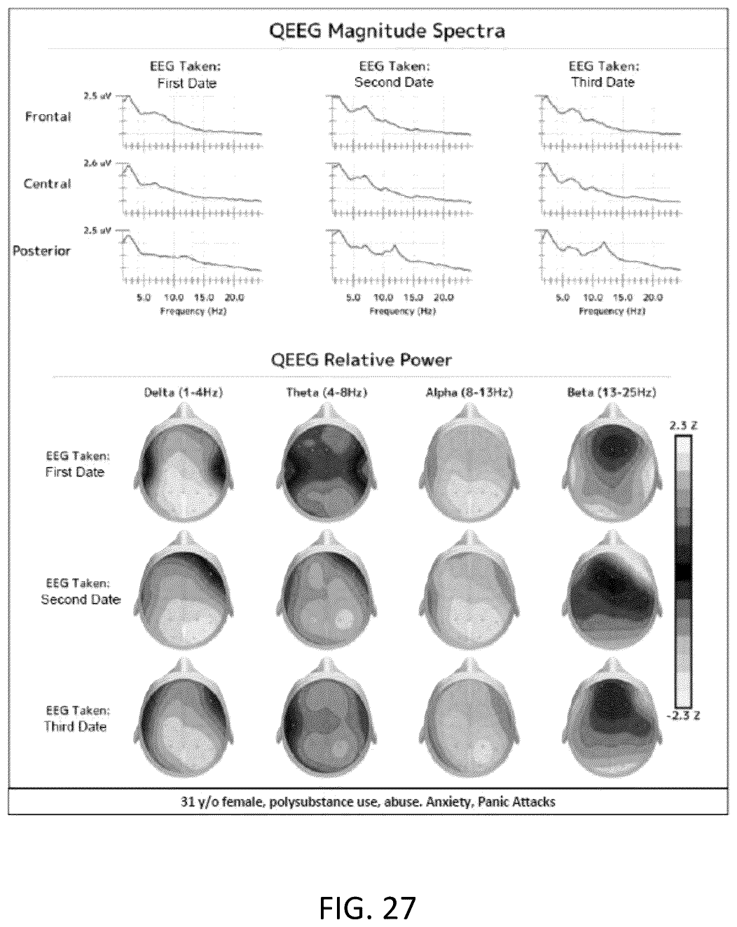

FIG. 27 shows relative power that may be indicative of a brain type called “low voltage alpha”. Low voltage alpha may be a normal phenotype, occurring in about 5-10% of the population. In example embodiments, low voltage alpha may be indicated by a combination of QEEG brain maps with high delta, high theta, low alpha, and high beta. In FIG. 27, the shading indicates the different voltages for alpha, delta, theta, and beta. This combination may be referred to herein as a “low voltage alpha signature”. A low voltage alpha signature may have relatively low amplitude in the alpha band (about 8 Hz to about 13 Hz). As disclosed herein, a brain with a low voltage alpha signature may be less responsive to neuromodulation, and may require an additional place on the head for brain simulation.

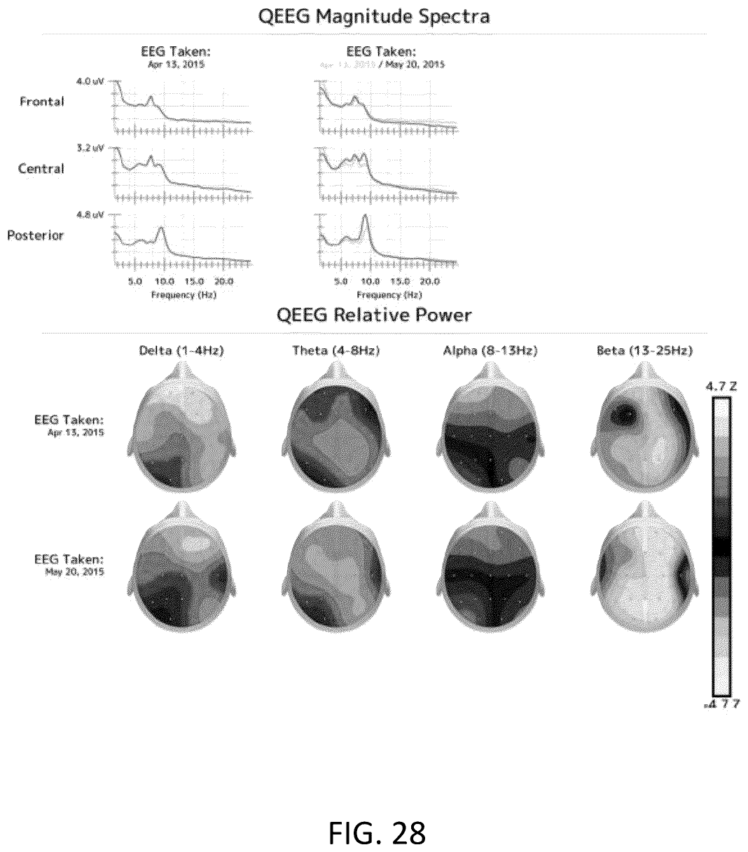

FIG. 28 shows a pre/post stroke case before and after about 40 sessions of MeRT therapy. The stroke occurred on the right side of the head of a 63-year-old male. In stroke cases, a spot or region of higher energy as depicted in delta (see, for example, FIG. 28) may be indicative of where a stroke occurred. If the stroke is hemorrhagic or ischemic and/or if the stroke significantly influences a function, even 10 years after the stroke, a same spot or region may be indicated as having higher relative power in the delta or theta QEEG relative power brain maps. Charts as shown in FIG. 28 may be indicative of how a stroke victim's functions may be influenced. As disclosed herein, foci of interference of ideal function may be a target for TMS.

In example embodiments, elevated theta may be indicative of a brain injury. Theta is shown in the second QEEG brain map from the left in FIG. 28. In FIG. 28, the QEEG magnitude spectra shows a deviation from a synchronous peak on the QEEG magnitude spectra shown in FIG. 25; there is a double peak in the theta range in FIG. 28 that is particularly evident in the Central QEEG magnitude spectrum.

All of the information in FIG. 28 may help to visualize injuries that may influence function. FIG. 28 may allow for easy visualization of changes over time.

Still referring to FIG. 28, the top of the page shows QEEG magnitude spectra (FFTs). There may be space provisioned to the right of the FFT for showing QEEG magnitude spectra for subsequent sessions. The QEEG magnitude spectra shown on the right in FIG. 28 superimposes QEEG magnitude spectra from after TMS treatment over the QEEG magnitude spectra from before TMS treatment. The QEEG magnitude spectra from after TMS treatment may have greater amplitude than the QEEG magnitude spectra from before TMS treatment, particularly in the central and posterior spectra. The changes in the QEEG before and after TMS may also be visible in the QEEG relative power diagrams. The QEEG relative power diagrams show decreases in Delta and Beta, and increases in Theta and Alpha. Improvement was reported in cognition, speech, processing speed, and sleep.

The records are depicted at the same scale to allow for relatively easy comparison of records over time. In example embodiments, brain rhythms and their changes over time may be recorded in a drawing (e.g., as shown in FIG. 28).

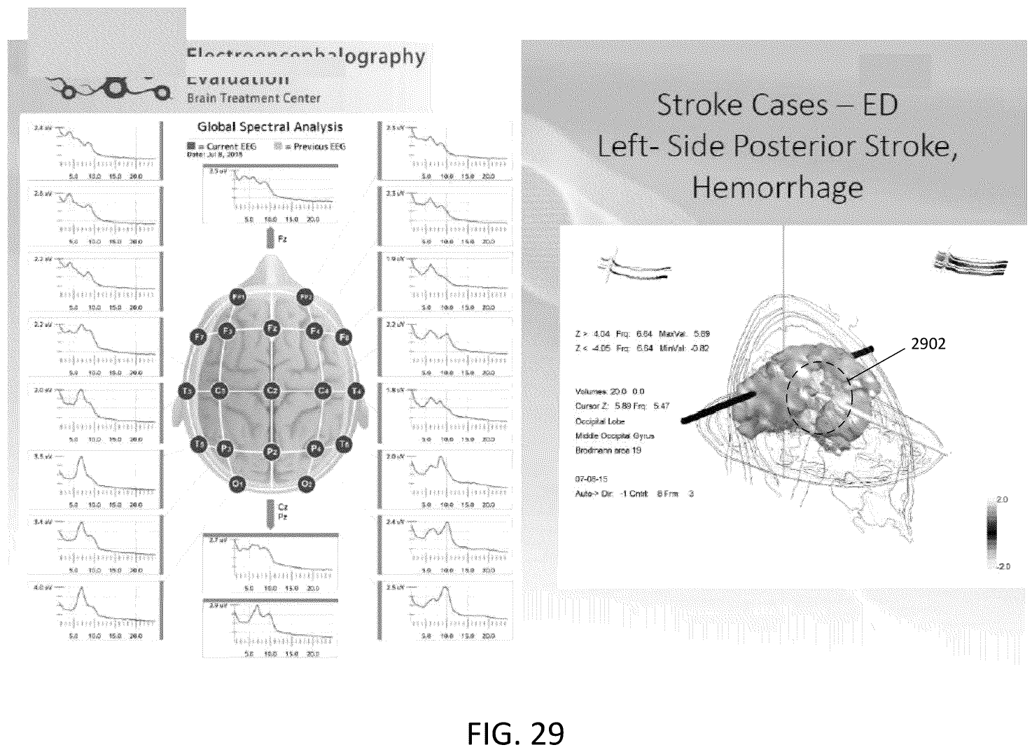

FIG. 29 is a head of FFTs that depicts a peak in the lower left-hand corner where a stroke was, and that peaks at about 7 Hz. On the right of FIG. 29 is a standardized low-resolution brain electromagnetic tomography (sLORETA) diagram that shows three dimensionally where the high activity may be located. FIG. 29 represents a victim of a stroke in a left posterior part of the brain indicated by the region 2902 in the left posterior part of the sLORETA image in FIG. 29. On the right side of the brain where there was no injury, the peak is at about 10 Hz. The unimpaired area may be working at about 10 Hz, and the impaired area may work slower.

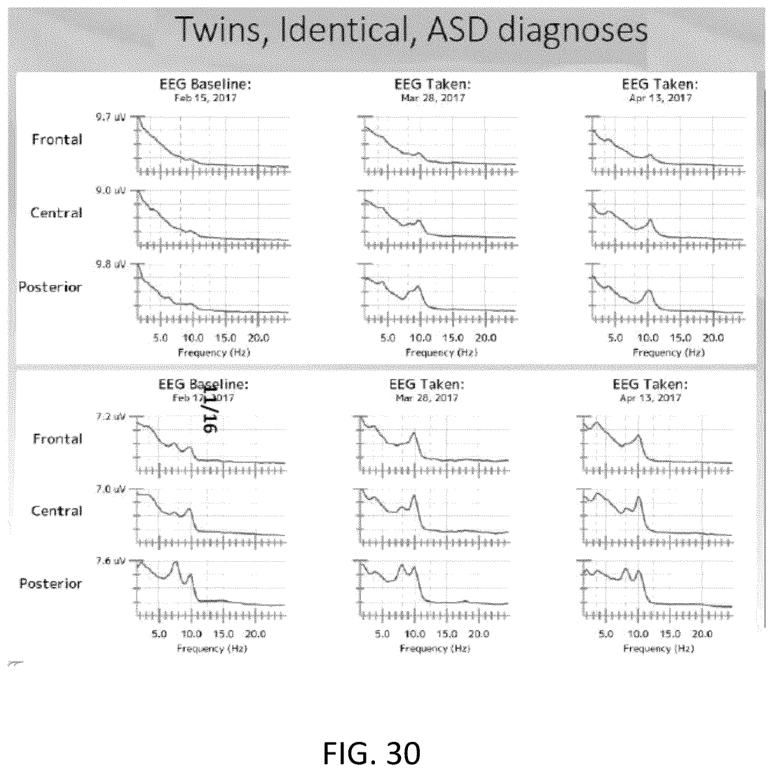

As disclosed herein, the EEGs of autistic children may show a significant deviation from normal ranges. FIG. 30 is a series of QEEG magnitude spectra from EEGs of identical twins. Both of the identical twin subjects of FIG. 30 had reported developmental delay, speech delay, and cognitive delay. Both sets of QEEG magnitude spectra in FIG. 30 show reductions in activities in alpha (about 8 Hz-about 13 Hz) ranges, as well as relatively lower frontal density of alpha activity. Following about one month of therapy, both of the identical twins reported improvements in cognition, attention, and language (as shown in the EEGs Taken Mar. 28, 2017). Even though identical twins have nearly the same DNA, the EEGs of identical twins may exhibit differences. Identical twins may also respond differently to therapy. The set of QEEG magnitude spectra from the identical twin shown on the bottom has another peak at about eight that is not significantly diminished in the second and third sessions. The set of QEEG magnitude spectra from the identical twin shown on top has a peak at about 10 Hz. The trajectory response of these twins with such differences in the QEEG magnitude spectra may tend to be different.

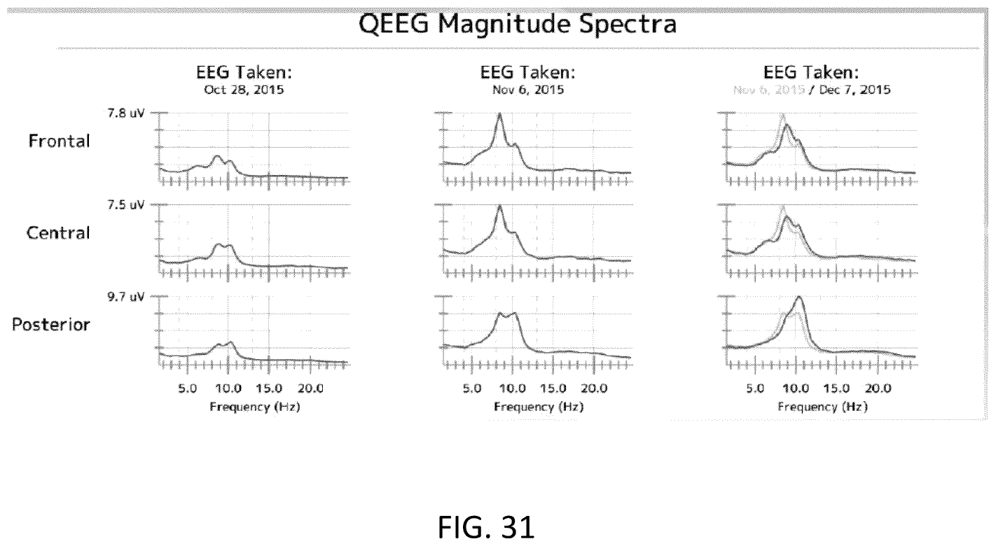

FIG. 31 shows another case involving stroke. QEEG magnitude spectra are shown from before treatment, after about one week of treatment and after about five weeks of treatment.

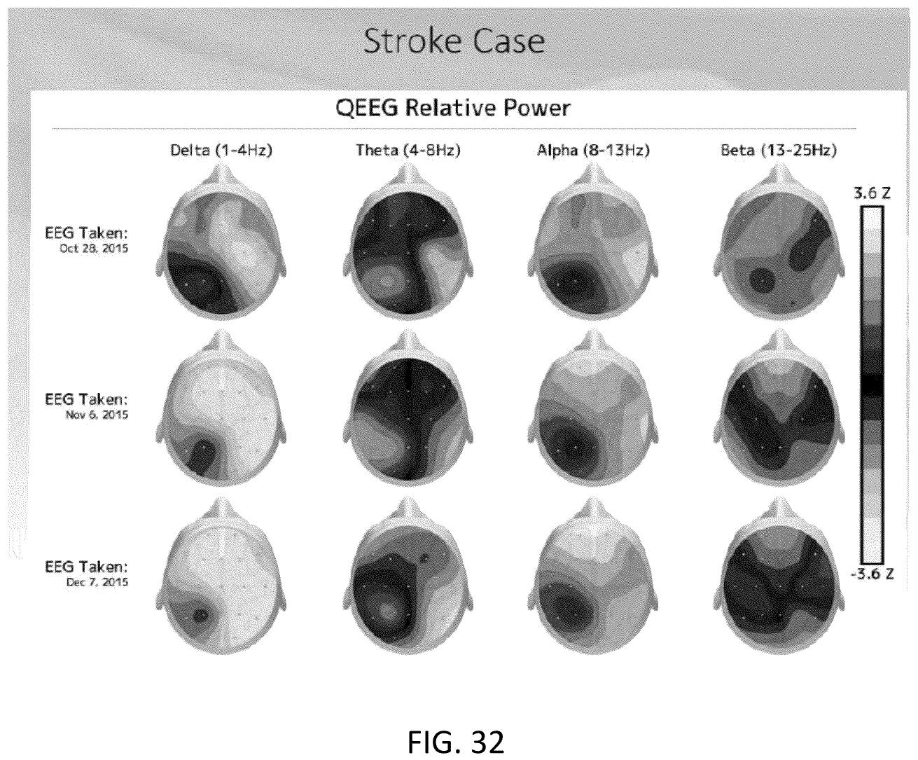

FIG. 32 depicts QEEG relative power diagrams from the same case that is shown in FIG. 31. The diagrams shown in FIG. 32 are from a follow-up evaluation after two months of therapy (about 31 MeRT sessions). Improvements in speech, motivation, and sleep were reported. Mild improvement in sensation was reported. There is a spot that may indicate a focus of stroke in the left posterior artery, indicated by elevated delta. Referring to the alpha diagrams, the spot in the left posterior region shrinks over time, and the alpha diagrams have more power over time. As indicated in the scale on the right, power is indicated in the QEEG relative power diagrams based on shading.

In example embodiments, the EEG analytic processing, as described in the disclosure, may have better noise immunity than other EEG analytic processes. The noise immunity may be advantageous because some subjects may move their heads during data collection for the EEG.

In example embodiments, the KDE plots disclosed herein may have particular advantage in cases involving autism because autistic individuals, particularly autistic children, may generate movement artifacts in the EEG. In example embodiments, burst statistics may be gleaned from the EEG that are independent of the movement-induced noise.

In example embodiments, stimulating a resonant response may be accomplished by electromagnetic modulation, light modulation, touch, rhythmic activity, or sound modulation.

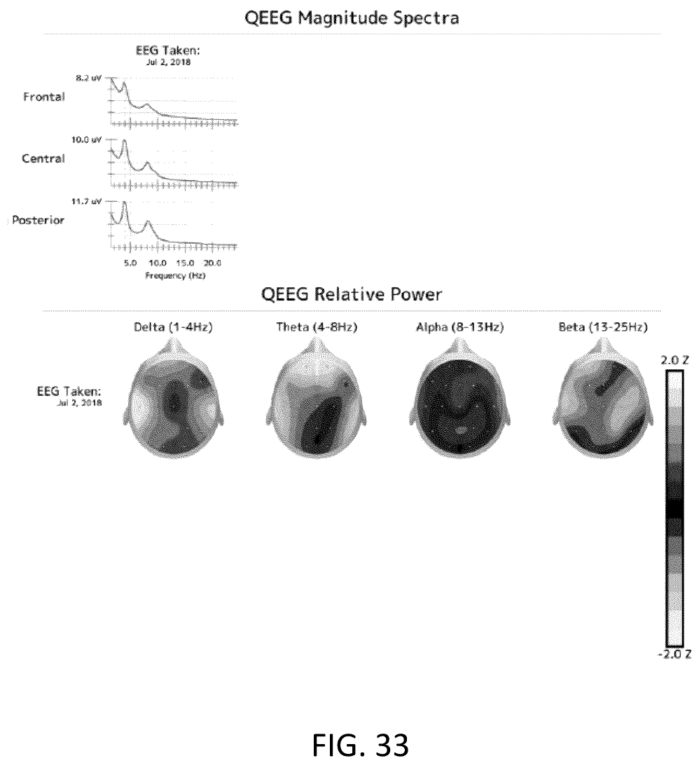

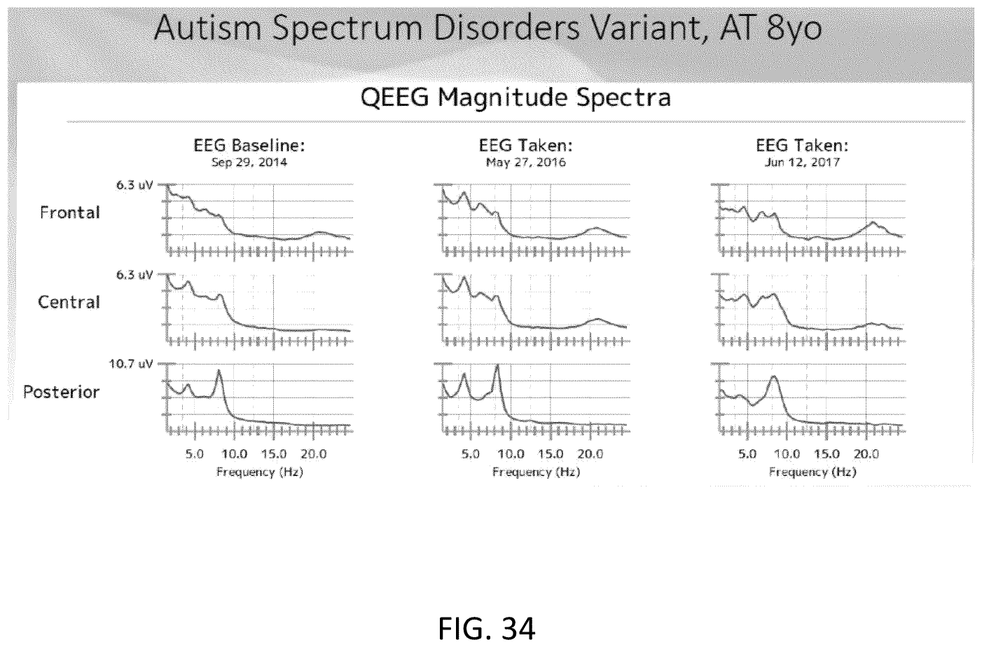

As shown in FIG. 33, in example embodiments, an EEG characteristic at about 4 Hz to about 4.5 Hz out of left and right temporal regions may be indicative of a mutation in the MTHFR gene. In some example embodiments, the determined peak energy may be initially closer to about 4 Hz (e.g., initially, and early in therapy, peak may be relatively lower and closer to about 4 Hz). Eventually, the peak energy may be determined as being closer to about 4.5 Hz. In FIG. 33, interspersed activity at about 4 Hz may indicate a possibility of metabolic/MTHFR pathway disruption. Two main mutations of the MTHFR gene have been reported: C677T and A1298C. In example embodiments, confirmatory tests for MTHFR polymorphisms may be recommended after a peak is detected in a range from about 4 Hz to about 4.5 Hz from the left and right temporal regions. Certain dietary supplements that increase folate may be available to reduce the peak in the range from about 4 Hz to about 4.5 Hz. As disclosed herein, when the peak in the range from about 4 Hz to about 4.5 Hz is reduced, cognition may improve. FIG. 34 is based on data from a 5-year-old male with high functioning autism spectrum disorder (ASD). The Childhood Autism Rating Scale (CARS) was about 25, and the Global Assessment of Functioning (GAF) was about 40. FIG. 33 shows parietal-foci, 4 Hz alpha activity. No developmental abnormalities were noted. FIG. 33 shows about 9 Hz dominant EEG activity globally.

FIG. 34 shows the peak in the range from about 4 Hz to about 4.5 Hz that is associated with the L-methylfolate mutation. FIG. 34 shows QEEG magnitude spectra from tests on a patient who received magnetic resonance therapy (MeRT)over a period of about 2 years. The peak in the range from about 4 Hz-about 4.5 Hz is visible in the middle set of QEEG magnitude spectra taken at the end of about 2 years of MeRT. The set of QEEG magnitude spectra on the right was made after MeRT was discontinued, and only dietary supplementation to increase folate was provided. In the set of QEEG magnitude spectra on the right, the peak in the range from about 4 Hz-about 4.5 Hz is shown to be significantly diminished.

In example embodiments, an EEG characteristic in a range from about 4 Hz-about 4.5 Hz out of left and right temporal regions may be indicative of a condition that may be improved by dietary supplementation with L methylfolate.

As disclosed herein, in cases of Alzheimer's and dementia, a flickering light may be helpful in a fast frequency. The effects of light may fade relatively quickly but light may be helpful in some treatment combinations. If a patient is exposed to flickering light at an individualized alpha frequency (treatment frequency), and simultaneously given MeRT or neuromodulation, the effect may be enhanced over a sum of the effects of the flickering light and the neuromodulation administered one at a time. Multimodal stimulation may help with Lewy body dementia (LBD). In example embodiments, exposing a subject to a strobe light that may match an IAF may be provided simultaneously with neuromodulation for some low voltage cases, because low voltage cases may have relatively more difficulty in generating this facilitatory activity. Without being held bound to any theory, it is believed that multimodal stimulation may rouse the brain to generate the facilitatory activity on its own with the strobe, and the neuromodulation may make the effect last longer.

In example embodiments, the brain may be stimulated by at least one of alternating magnetic fields, transcranial electrical stimulation, ultrasound, functional near infrared spectroscopy, or sensory stimulation. The sensory stimulation may include at least one of flashing light, sound, video, or touch.

In example embodiments, a slower EEG may be indicative of a glutamate deficiency. Glutamate may be an excitatory nerve transmitter. For example, in response to a slower EEG, a recommendation may be made to take supplements that may increase glutamate. In example embodiments, an EEG that shows a relatively high beta, e.g., a relatively more excitable, relatively more chaotic EEG, may be indicative of a Gamma-aminobutyric acid (GABA) deficiency. GABA may be an amino acid that serves as the primary inhibitory neurotransmitter in the brain and a major inhibitory neurotransmitter in the spinal cord. For those who may have differences in being able to generate oscillatory activity, it may indicate that relatively more acetylcholine may be beneficial.

In example embodiments, a full treatment paradigm for improving brain function may include TMS, magnetic fields, supplements, medication, light, and/or sunlight.

In example embodiments, there may be different brain types, different start points for people, and/or different underlying architectures. Understanding the start points, understanding the underlying architecture for an individual may help determine a relatively more personalized approach based off of the underlying architecture. In example embodiments, the detected underlying architecture may be compared to an underlying ideal architecture for that person to provide effective treatment that may be tailored to the individual. The brain comprises many functional “networks” of neurons, which are interconnected clusters that are active during a particular task. For example, neurons in the occipital lobe are active during the task of receiving visual information. The auditory cortex is active when listening to sounds. The motor cortex is active when purposely moving one or more parts of the body. When no task is being performed, the brain can be thought of in an idle functional state.

These functional networks are found using, for example, fMRI, EEG, MEG, PET, or SPECT. In some embodiments, the functional networks are found through cortical mapping using, for example, TMS pulses, direct cortical stim, or peripheral stim. In some embodiments, functional networks are found while the person is activating the functional network in the brain. The functional network of the brain is activated by performing a functional task associated with the functional network. In some embodiments, the functional task is actually performed (e.g., by moving an arm) to activate the functional network. In some embodiments, the individual envisions themselves performing the functional task without actually performing it to activate the functional network (e.g., thinking about moving an arm, visualizing movement of an arm, etc.). In some embodiments, a battery of tests (e.g., tasks) are used to activate or maintain activity of the functional network.

Once the functional network is found, the EEG of the individual is recorded during the functional task (or while thinking about the functional task or visualizing the functional task). As described above, the EEG is recorded using electrodes that are electrically connected to the individual's scalp. In some examples, the electrodes are positioned close to (or near) the function network(s) of interest. In some embodiments, if an EEG cap is used comprising many scalp electrodes, only the EEG signals from electrodes close to (or near) the functional network(s) are used. Alternatively, a weighting scheme is used (e.g., by one or more algorithms) to emphasize the signal(s) from electrodes close to the functional network(s). For example, specific channels, signals, and/or frequencies are weighted to emphasize the collected data associated with the functional network(s). In some embodiments, filters (e.g., digital or physical) are used to emphasize the signal(s) from electrodes close to the functional network(s).

The frequency content of the EEG signals is calculated, and one or more intrinsic EEG frequencies of an EEG band is determined. In some embodiments, the EEG band corresponds to the In some embodiments, stimulation is provided to the individual to change one or more characteristics of the intrinsic EEG frequencies of the functional network(s). In some embodiments, stimulation is provided using rTMS, tACS, tDCS, ultrasound, rhythmic sensory stim, vibration, or some other means. In some embodiments, the stimulation is provided at a stimulation frequency set to one or more of the intrinsic EEG frequencies. In some embodiments, stimulation is provided so that the maximum stimulation intensity is located at or near the region of the functional network. In some embodiments, stimulation is administered with the individual at rest. In some embodiments, stimulation is administered while the individual is performing one or more functional tasks, or thinking about performing the one or more functional tasks.

In some embodiments, the functional network is re-determined (or re-located) following one or more stimulation sessions. For example, in some embodiments additional brain regions are recruited based on the stimulation and functional training, or regions of the functional network are shifted or reduced in size. After this re-determination, the EEG is re-recorded, the intrinsic EEG frequencies are re-calculated, and the stimulation parameters are adjusted.

In some embodiments, stimulation improves the individual's ability to perform functional tasks associated with the functional network(s). In some embodiments, the individual's performance during the functional tasks is monitored before and after stimulation to measure (or observe) improvements. In some embodiments, the individual's performance before and/or after stimulation is compared to the individual's performance while receiving stimulation and performing the functional tasks simultaneously. In some embodiments, closed loop feedback is used to jumpstart and/or prime functional networks of the individual.

The background description is presented simply for context, and is not necessarily well-understood, routine, or conventional. Further, the background description is not an admission of what does or does not qualify as prior art. In fact, some or all of the background description may be work attributable to the named inventors that is otherwise unknown in the art.

Physical (such as spatial and/or electrical) and functional relationships between elements (for example, between modules, circuit elements, semiconductor layers, etc.) are described using various terms. Unless explicitly described as being “direct,” when a relationship between first and second elements is described, that relationship encompasses both (i) a direct relationship where no other intervening elements are present between the first and second elements and (ii) an indirect relationship where one or more intervening elements are present between the first and second elements.

Example relationship terms include “adjoining,” “transmitting,” “receiving,” “connected,” “engaged,” “coupled,” “adjacent,” “next to,” “on top of,” “above,” “below,” “abutting,” and “disposed.”

The detailed description includes specific examples for illustration only, and not to limit the disclosure or its applicability. The examples are not intended to be an exhaustive list, but instead simply demonstrate possession by the inventors of the full scope of the currently presented and envisioned future claims. Variations, combinations, and equivalents of the examples are within the scope of the disclosure.

No language in the specification should be construed as indicating that any non-claimed element is essential or critical to the practice of the disclosure.

The term “exemplary” simply means “example” and does not indicate a best or preferred example.

The term “set” does not necessarily exclude the empty set in other words, in some circumstances a “set” may have zero elements. The term “non-empty set” may be used to indicate exclusion of the empty set that is, a non-empty set must have one or more elements.

The term “subset” does not necessarily require a proper subset. In other words, a “subset” of a first set may be coextensive with (equal to) the first set. Further, the term “subset” does not necessarily exclude the empty set in some circumstances a “subset” may have zero elements.

The phrase “at least one of A, B, and C” should be construed to mean a logical (A OR B OR C), using a non-exclusive logical OR, and should not be construed to mean “at least one of A, at least one of B, and at least one of C.”

The use of the terms “a,” “an,” “the,” and similar referents in the context of describing the disclosure and claims encompasses both the singular and the plural, unless contradicted explicitly or by context.

Unless otherwise specified, the terms “comprising,” “having,” “with,” “including,” and “containing,” and their variants, are open-ended terms, meaning “including, but not limited to.”

Each publication referenced in this disclosure, including foreign and domestic patent applications and patents, is hereby incorporated by reference in its entirety.

Although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of multiple embodiments remain within the scope of this disclosure.

One or more elements (for example, steps within a method, instructions, actions, or operations) may be executed in a different order (and/or concurrently) without altering the principles of the present disclosure.

Unless technically infeasible, elements described as being in series may be implemented partially or fully in parallel. Similarly, unless technically infeasible, elements described as being in parallel may be implemented partially or fully in series.

While the disclosure describes structures corresponding to claimed elements, those elements do not necessarily invoke a means plus function interpretation unless they explicitly use the signifier “means for.”

While the drawings divide elements of the disclosure into different functional blocks or action blocks, these divisions are for illustration only. According to the principles of the present disclosure, functionality can be combined in other ways such that some or all functionality from multiple separately-depicted blocks can be implemented in a single functional block; similarly, functionality depicted in a single block may be separated into multiple blocks.

Unless explicitly stated as mutually exclusive, features depicted in different drawings can be combined consistent with the principles of the present disclosure.

In the drawings, reference numbers may be reused to identify identical elements or may simply identify elements that implement similar functionality.

Numbering or other labeling of instructions or method steps is done for convenient reference, not to indicate a fixed order.

In the drawings, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. As just one example, for information sent from element A to element B, element B may send requests and/or acknowledgments to element A.

Unless otherwise indicated, recitations of ranges of values are merely intended to serve as a shorthand way of referring individually to each separate value falling within the range, and each separate value is hereby incorporated into the specification as if it were individually recited.

Special-Purpose System

A special-purpose system includes hardware and/or software and may be described in terms of an apparatus, a method, or a computer-readable medium. In various embodiments, functionality may be apportioned differently between software and hardware. For example, some functionality may be implemented by hardware in one embodiment and by software in another embodiment. Further, software may be encoded by hardware structures, and hardware may be defined by software, such as in software-defined networking or software-defined radio.

In this application, including the claims, the term module refers to a special-purpose system. The module may be implemented by one or more special-purpose systems. The one or more special-purpose systems may also implement some or all of the other modules.

In this application, including the claims, the term module may be replaced with the terms controller or circuit.

In this application, including the claims, the term platform refers to one or more modules that offer a set of functions.

In this application, including the claims, the term system may be used interchangeably with module or with the term special-purpose system.

The special-purpose system may be directed or controlled by an operator. The special-purpose system may be hosted by one or more of assets owned by the operator, assets leased by the operator, and third-party assets. The assets may be referred to as a private, community, or hybrid cloud computing network or cloud computing environment.

For example, the special-purpose system may be partially or fully hosted by a third-party offering software as a service (SaaS), platform as a service (PaaS), and/or infrastructure as a service (IaaS).

The special-purpose system may be implemented using agile development and operations (DevOps) principles. In embodiments, some or all of the special-purpose system may be implemented in a multiple-environment architecture. For example, the multiple environments may include one or more production environments, one or more integration environments, one or more development environments, etc.

### Device Examples

A special-purpose system may be partially or fully implemented using or by a mobile device. Examples of mobile devices include navigation devices, cell phones, smart phones, mobile phones, mobile personal digital assistants, palmtops, netbooks, pagers, electronic book readers, tablets, music players, etc.

A special-purpose system may be partially or fully implemented using or by a network device. Examples of network devices include switches, routers, firewalls, gateways, hubs, base stations, access points, repeaters, head-ends, user equipment, cell sites, antennas, towers, etc.

A special-purpose system may be partially or fully implemented using a computer having a variety of form factors and other characteristics. For example, the computer may be characterized as a personal computer, as a server, etc. The computer may be portable, as in the case of a laptop, netbook, etc. The computer may or may not have any output device, such as a monitor, line printer, liquid crystal display (LCD), light emitting diodes (LEDs), etc. The computer may or may not have any input device, such as a keyboard, mouse, touchpad, trackpad, computer vision system, barcode scanner, button array, etc. The computer may run a general-purpose operating system, such as the WINDOWS operating system from Microsoft Corporation, the MACOS operating system from Apple, Inc., or a variant of the LINUX operating system.

Examples of servers include a file server, print server, domain server, internet server, intranet server, cloud server, infrastructure-as-a-service server, platform-as-a-service server, web server, secondary server, host server, distributed server, failover server, and backup server.

Hardware

The term hardware encompasses components such as processing hardware, storage hardware, networking hardware, and other general-purpose and special-purpose components. Note that these are not mutually-exclusive categories. For example, processing hardware may integrate storage hardware and vice versa.

Examples of a component are integrated circuits (ICs), application specific integrated circuit (ASICs), digital circuit elements, analog circuit elements, combinational logic circuits, gate arrays such as field programmable gate arrays (FPGAs), digital signal processors (DSPs), complex programmable logic devices (CPLDs), etc.

Multiple components of the hardware may be integrated, such as on a single die, in a single package, or on a single printed circuit board or logic board. For example, multiple components of the hardware may be implemented as a system-on-chip. A component, or a set of integrated components, may be referred to as a chip, chipset, chiplet, or chip stack.

Examples of a system-on-chip include a radio frequency (RF) system-on-chip, an artificial intelligence (AI) system-on-chip, a video processing system-on-chip, an organ-on-chip, a quantum algorithm system-on-chip, etc.

The hardware may integrate and/or receive signals from sensors. The sensors may allow observation and measurement of conditions including temperature, pressure, wear, light, humidity, deformation, expansion, contraction, deflection, bending, stress, strain, load-bearing, shrinkage, power, energy, mass, location, temperature, humidity, pressure, viscosity, liquid flow, chemical/gas presence, sound, and air quality. A sensor may include image and/or video capture in visible and/or non-visible (such as thermal) wavelengths, such as a charge-coupled device (CCD) or complementary metal-oxide semiconductor (CMOS) sensor.

Processing Hardware

Examples of processing hardware include a central processing unit (CPU), a graphics processing unit (GPU), an approximate computing processor, a quantum computing processor, a parallel computing processor, a neural network processor, a signal processor, a digital processor, a data processor, an embedded processor, a microprocessor, and a co-processor. The co-processor may provide additional processing functions and/or optimizations, such as for speed or power consumption. Examples of a co-processor include a math co-processor, a graphics co-processor, a communication co-processor, a video co-processor, and an artificial intelligence (AI) co-processor.

Processor Architecture

The processor may enable execution of multiple threads. These multiple threads may correspond to different programs. In various embodiments, a single program may be implemented as multiple threads by the programmer or may be decomposed into multiple threads by the processing hardware. The threads may be executed simultaneously to enhance the performance of the processor and to facilitate simultaneous operations of the application.

A processor may be implemented as a packaged semiconductor die. The die includes one or more processing cores and may include additional functional blocks, such as cache. In various embodiments, the processor may be implemented by multiple dies, which may be combined in a single package or packaged separately.

Networking Hardware

The networking hardware may include one or more interface circuits. In some examples, the interface circuit(s) may implement wired or wireless interfaces that connect, directly or indirectly, to one or more networks. Examples of networks include a cellular network, a local area network (LAN), a wireless personal area network (WPAN), a metropolitan area network (MAN), and/or a wide area network (WAN). The networks may include one or more of point-to-point and mesh technologies. Data transmitted or received by the networking components may traverse the same or different networks. Networks may be connected to each other over a WAN or point-to-point leased lines using technologies such as Multiprotocol Label Switching (MPLS) and virtual private networks (VPNs).

Examples of cellular networks include GSM, GPRS, 3G, 4G, 5G, LTE, and EVDO. The cellular network may be implemented using frequency division multiple access (FDMA) network or code division multiple access (CDMA) network.

Examples of a LAN are Institute of Electrical and Electronics Engineers (IEEE) Standard 802.11-2020 (also known as the WIFI wireless networking standard) and IEEE Standard 802.3-2018 (also known as the ETHERNET wired networking standard).

Examples of a WPAN include IEEE Standard 802.15.4, including the ZIGBEE standard from the ZigBee Alliance. Further examples of a WPAN include the BLUETOOTH wireless networking standard, including Core Specification versions 3.0, 4.0, 4.1, 4.2, 5.0, and 5.1 from the Bluetooth Special Interest Group (SIG).

A WAN may also be referred to as a distributed communications system (DCS). One example of a WAN is the internet.

Storage Hardware

Storage hardware is or includes a computer-readable medium. The term computer-readable medium, as used in this disclosure, encompasses both nonvolatile storage and volatile storage, such as dynamic random access memory (DRAM). The term computer-readable medium only excludes transitory electrical or electromagnetic signals propagating through a medium (such as on a carrier wave). A computer-readable medium in this disclosure is therefore non-transitory, and may also be considered to be tangible.

### EXAMPLES

Examples of storage implemented by the storage hardware include a database (such as a relational database or a NoSQL database), a data store, a data lake, a column store, a data warehouse.

Examples of storage hardware include nonvolatile memory devices, volatile memory devices, magnetic storage media, a storage area network (SAN), network-attached storage (NAS), optical storage media, printed media (such as bar codes and magnetic ink), and paper media (such as punch cards and paper tape). The storage hardware may include cache memory, which may be collocated with or integrated with processing hardware.

Storage hardware may have read-only, write-once, or read/write properties. Storage hardware may be random access or sequential access. Storage hardware may be location-addressable, file-addressable, and/or content-addressable.

Examples of nonvolatile memory devices include flash memory (including NAND and NOR technologies), solid state drives (SSDs), an erasable programmable read-only memory device such as an electrically erasable programmable read-only memory (EEPROM) device, and a mask read-only memory device (ROM).

Examples of volatile memory devices include processor registers and random access memory (RAM), such as static RAM (SRAM), dynamic RAM (DRAM), synchronous DRAM (SDRAM), synchronous graphics RAM (SGRAM), and video RAM (VRAM).

Examples of magnetic storage media include analog magnetic tape, digital magnetic tape, and rotating hard disk drive (HDDs).

Examples of optical storage media include a CD (such as a CD-R, CD-RW, or CD-ROM), a DVD, a Blu-ray disc, and an Ultra HD Blu-ray disc.

Examples of storage implemented by the storage hardware include a distributed ledger, such as a permissioned or permissionless blockchain.

Entities recording transactions, such as in a blockchain, may reach consensus using an algorithm such as proof-of-stake, proof-of-work, and proof-of-storage.

Elements of the present disclosure may be represented by or encoded as non-fungible tokens (NFTs). Ownership rights related to the non-fungible tokens may be recorded in or referenced by a distributed ledger.

Transactions initiated by or relevant to the present disclosure may use one or both of fiat currency and cryptocurrencies, examples of which include bitcoin and ether.

Some or all features of hardware may be defined using a language for hardware description, such as IEEE Standard 1364-2005 (commonly called “Verilog”) and IEEE Standard 1076-2008 (commonly called “VHDL”). The hardware description language may be used to manufacture and/or program hardware.

A special-purpose system may be distributed across multiple different software and hardware entities. Communication within a special-purpose system and between special-purpose systems may be performed using networking hardware. The distribution may vary across embodiments and may vary over time. For example, the distribution may vary based on demand, with additional hardware and/or software entities invoked to handle higher demand. In various embodiments, a load balancer may direct requests to one of multiple instantiations of the special purpose system. The hardware and/or software entities may be physically distinct and/or may share some hardware and/or software, such as in a virtualized environment. Multiple hardware entities may be referred to as a server rack, server farm, data center, etc.

Software

Software includes instructions that are machine-readable and/or executable. Instructions may be logically grouped into programs, codes, methods, steps, actions, routines, functions, libraries, objects, classes, etc. Software may be stored by storage hardware or encoded in other hardware. Software encompasses (i) descriptive text to be parsed, such as HTML (hypertext markup language), XML (extensible markup language), and JSON (JavaScript Object Notation), (ii) assembly code, (iii) object code generated from source code by a compiler, (iv) source code for execution by an interpreter, (v) bytecode, (vi) source code for compilation and execution by a just-in-time compiler, etc. As examples only, source code may be written using syntax from languages including C, C++, JavaScript, Java, Python, R, etc.

Software also includes data. However, data and instructions are not mutually-exclusive categories. In various embodiments, the instructions may be used as data in one or more operations. As another example, instructions may be derived from data.

The functional blocks and flowchart elements in this disclosure serve as software specifications, which can be translated into software by the routine work of a skilled technician or programmer.

Software may include and/or rely on firmware, processor microcode, an operating system (OS), a basic input/output system (BIOS), application programming interfaces (APIs), libraries such as dynamic-link libraries (DLLs), device drivers, hypervisors, user applications, background services, background applications, etc. Software includes native applications and web applications. For example, a web application may be served to a device through a browser using hypertext markup language 5th revision (HTML5).

Software may include artificial intelligence systems, which may include machine learning or other computational intelligence. For example, artificial intelligence may include one or more models used for one or more problem domains.

When presented with many data features, identification of a subset of features that are relevant to a problem domain may improve prediction accuracy, reduce storage space, and increase processing speed. This identification may be referred to as feature engineering. Feature engineering may be performed by users or may only be guided by users. In various implementations, a machine learning system may computationally identify relevant features, such as by performing singular value decomposition on the contributions of different features to outputs.

Examples of the models include recurrent neural networks (RNNs) such as long short-term memory (LSTM), deep learning models such as transformers, decision trees, support-vector machines, genetic algorithms, Bayesian networks, and regression analysis. Examples of systems based on a transformer model include bidirectional encoder representations from transformers (BERT) and generative pre-trained transformer (GPT).

Training a machine-learning model may include supervised learning (for example, based on labeled input data), unsupervised learning, and reinforcement learning. In various embodiments, a machine-learning model may be pre-trained by their operator or by a third party.

Problem domains include nearly any situation where structured data can be collected, and includes natural language processing (NLP), computer vision (CV), classification, image recognition, etc.

Architectures

Some or all of the software may run in a virtual environment rather than directly on hardware. The virtual environment may include a hypervisor, emulator, sandbox, container engine, etc. The software may be built as a virtual machine, a container, etc. Virtualized resources may be controlled using, for example, a DOCKER container platform, a pivotal cloud foundry (PCF) platform, etc.

In a client-server model, some of the software executes on first hardware identified functionally as a server, while other of the software executes on second hardware identified functionally as a client. The identity of the client and server is not fixed: for some functionality, the first hardware may act as the server while for other functionality, the first hardware may act as the client. In different embodiments and in different scenarios, functionality may be shifted between the client and the server. In one dynamic example, some functionality normally performed by the second hardware is shifted to the first hardware when the second hardware has less capability. In various embodiments, the term “local” may be used in place of “client,” and the term “remote” may be used in place of“server.”

Some or all of the software may be logically partitioned into microservices. Each microservice offers a reduced subset of functionality. In various embodiments, each microservice may be scaled independently depending on load, either by devoting more resources to the microservice or by instantiating more instances of the microservice. In various embodiments, functionality offered by one or more microservices may be combined with each other and/or with other software not adhering to a microservices model.

Some or all of the software may be arranged logically into layers. In a layered architecture, a second layer may be logically placed between a first layer and a third layer. The first layer and the third layer would then generally interact with the second layer and not with each other. In various embodiments, this is not strictly enforced—that is, some direct communication may occur between the first and third layers.

1-62. (canceled)

63. A method for scoring and reporting electroencephalogram (EEG) data for use in transcranial magnetic stimulation (TMS) therapy comprising:
receiving EEG data collected from a subject;
removing artifacts from the EEG data;
determining a plurality of EEG metrics from the EEG data; and
determining a Brain Synchrony Score from the plurality of EEG metrics by applying a predetermined transfer function to the plurality of EEG metrics.

64. The method of claim 63, further comprising:
reporting, via a user interface, the Brain Synchrony Score.

65. The method of claim 63, wherein the Brain Synchrony Score represents a level of synchronous energy in the EEG data that is within an alpha EEG band, the alpha EEG band being approximately 8 Hz to approximately 13 Hz.

66. The method of claim 63, further comprising:
determining at least one of a current frequency, an optimal frequency range, and an interference score based on the EEG data; and
reporting, via a user interface, at least one of the current frequency, the optimal frequency range, and the interference score.

67. The method of claim 66, wherein the current frequency represents a dominant frequency of the subject.

68. The method of claim 66, wherein the optimal frequency range represents an optimal frequency range for the subject.

69. The method of claim 66, wherein the interference score represents an amount of energy in the EEG data that is within a theta EEG band, the theta EEG band being approximately 5 Hz to approximately 7 Hz.

70. The method of claim 63, wherein the EEG data includes a plurality of EEG channels, wherein each EEG channel corresponds to a scalp electrode used to record the EEG data.

71. The method of claim 63, wherein removing artifacts from the EEG data includes filtering out at least one of noise artifacts and eye-blink artifacts.

72. The method of claim 63, wherein the EEG metrics include an intrinsic alpha frequency based on a dominant frequency in an alpha EEG band, the alpha EEG band being approximately 8 Hz to approximately 13 Hz.

73. The method of claim 63, wherein the predetermined transfer function is generated from at least one factor analysis performed on EEG data collected from a plurality of different subjects.

74. The method of claim 73, wherein the EEG data collected from the plurality of different subjects includes EEG data collected before and after TMS treatment.

75. The method of claim 73, wherein the at least one factor analysis includes an exploratory factor analysis (EFA) and a confirmatory factor analysis (CFA).

76. A system for scoring and reporting electroencephalogram (EEG) data for use in transcranial magnetic stimulation (TMS) therapy, comprising:
at least one memory storing computer-executable instructions; and
at least one processor for executing the instructions stored on the memory, wherein execution of the instructions programs the at least one processor to perform operations comprising:
receiving EEG data collected from a subject;
removing artifacts from the EEG data;
determining a plurality of EEG metrics from the EEG data; and
determining a Brain Synchrony Score from the plurality of EEG metrics by applying a predetermined transfer function to the plurality of EEG metrics.

77. The system of claim 76, wherein execution of the instructions programs the at least one processor to perform operations further comprising:
reporting, via a user interface, the Brain Synchrony Score.

78. The system of claim 76, wherein the Brain Synchrony Score represents a level of synchronous energy in the EEG data that is within an alpha EEG band, the alpha EEG band being approximately 8 Hz to approximately 13 Hz.

79. The system of claim 76, wherein execution of the instructions programs the at least one processor to perform operations further comprising:
determining at least one of a current frequency, an optimal frequency range, and an interference score based on the EEG data; and
reporting, via a user interface, at least one of the current frequency, the optimal frequency range, and the interference score.

80. The system of claim 79, wherein the current frequency represents a dominant frequency of the subject.

81. The system of claim 79, wherein the optimal frequency range represents an optimal frequency range for the subject.

82. The system of claim 79, wherein the interference score represents an amount of energy in the EEG data that is within a theta EEG band, the theta EEG band being approximately 5 Hz to approximately 7 Hz.

83. The system of claim 76, wherein the EEG data includes a plurality of EEG channels, wherein each EEG channel corresponds to a scalp electrode used to record the EEG data.

84. The system of claim 76, wherein removing artifacts from the EEG data includes filtering out at least one of noise artifacts and eye-blink artifacts.

85. The system of claim 76, wherein the plurality of EEG metrics include an intrinsic alpha frequency based on a dominant frequency in an alpha EEG band, the alpha EEG band being approximately 8 Hz to approximately 13 Hz.

86. The system of claim 76, wherein the predetermined transfer function is generated from at least one factor analysis performed on EEG data collected from a plurality of different subjects.

87. The system of claim 86, wherein the EEG data collected from the plurality of different subjects includes EEG data collected before and after TMS treatment.

88. The system of claim 86, wherein the at least one factor analysis includes an exploratory factor analysis (EFA) and a confirmatory factor analysis (CFA).
